SEQUENCE LISTING (86 sequences)

SEQ ID NO: 1 - DNA, 21 bp, Artificial Sequence
  Synthetic construct,  PCR forward primer for Calcr gene
gcaacgctttcacttctgaga

SEQ ID NO: 2 - DNA, 21 bp, Artificial Sequence
  Synthetic construct, PCR reverse primer for Calcr gene
gttcccactgcattgtccaca

SEQ ID NO: 3 - DNA, 21 bp, Artificial Sequence
  Synthetic construct, PCR forward primer for Tnfrsf11a gene
cactcccaccctgagatttgt

SEQ ID NO: 4 - DNA, 19 bp, Artificial Sequence
  Synthetic construct, PCR reverse primer for Tnfrsf11a gene
catcgtctgcacggttctg

SEQ ID NO: 5 - DNA, 19 bp, Artificial Sequence
  Synthetic construct, PCR forward primer for Nfatc1 gene
gacccggagttcgacttcg

SEQ ID NO: 6 - DNA, 23 bp, Artificial Sequence
  Synthetic construct, PCR reverse primer for Nfatc1 gene.
tgacactaggggacacataactg

SEQ ID NO: 7 - DNA, 19 bp, Artificial Sequence
  Synthetic construct, PCR forward primer for Csf1 gene.
tgtcatcgagcctagtggc

SEQ ID NO: 8 - DNA, 20 bp, Artificial Sequence
  Synthetic construct, PCR reverse primer Csf1 gene
cgggagattcagggtccaag

SEQ ID NO: 9 - DNA, 19 bp, Artificial Sequence
  Synthetic construct, PCR forward primer for Itgav gene
ccgtggacttcttcgagcc

SEQ ID NO: 10 - DNA, 23 bp, Artificial Sequence
  Synthetic construct, PCR reverse primer for Itgav gene
ctgttgaatcaaactcaatgggc

SEQ ID NO: 11 - DNA, 20 bp, Artificial Sequence
  Synthetic construct, PCR forward primer for Fosl2 gene
ccagcagaagttccgggtag

SEQ ID NO: 12 - DNA, 21 bp, Artificial Sequence
  Synthetic construct, PCR reverse primer for Fosl2 gene
gtagggatgtgagcgtggata

SEQ ID NO: 13 - DNA, 22 bp, Artificial Sequence
  Synthetic construct, PCR forward primer for Sfpi1 gene
atgttacaggcgtgcaaaatgg

SEQ ID NO: 14 - DNA, 21 bp, Artificial Sequence
  Synthetic construct, PCR  reverse primer for Sfpi1 gene
tgatcgctatggctttctcca

SEQ ID NO: 15 - DNA, 22 bp, Artificial Sequence
  Synthetic construct, PCR forward primer for Mitf gene
actttcccttatcccatccacc

SEQ ID NO: 16 - DNA, 22 bp, Artificial Sequence
  Synthetic construct, PCR reverse primer for Mitf gene
tgagatccagagttgtcgtaca

SEQ ID NO: 17 - DNA, 22 bp, Artificial Sequence
  Synthetic construct, PCR forward primer for Ctsk gene
gaagaagactcaccagaagcag

SEQ ID NO: 18 - DNA, 21 bp, Artificial Sequence
  Synthetic construct, PCR reverse primer for Ctsk gene
tccaggttatgggcagagatt

SEQ ID NO: 19 - DNA, 21 bp, Artificial Sequence
  Synthetic construct, PCR forward primer for Gapdh gene
aggtcggtgtgaacggatttg

SEQ ID NO: 20 - DNA, 23 bp, Artificial Sequence
  Synthetic construct, PCR reverse primer for Gapdh gene
tgtagaccatgtagttgaggtca

SEQ ID NO: 21 - DNA, 23 bp, Artificial Sequence
  Synthetic construct, PCR forward primer for 3′UTR of Calcr gene
aacacagcatcgtgatcactgag

SEQ ID NO: 22 - DNA, 24 bp, Artificial Sequence
  Synthetic construct, PCR reverse primer for 3′UTR of Calcr gene
cataatttgggcagaactatgtgc

SEQ ID NO: 23 - DNA, 22 bp, Artificial Sequence
  Synthetic construct, PCR forward primer for 3′UTR Acp5 gene
acaccacgagagtcctgcttgt

SEQ ID NO: 24 - DNA, 27 bp, Artificial Sequence
  Synthetic construct, PCR reverse primer for 3′UTR of Acp5 gene
ttctggagacaaagcattgttttattg

SEQ ID NO: 25 - DNA, 22 bp, Artificial Sequence
  Synthetic construct, PCR forward primer for 3′UTR of Mmp9 gene
agggctccttctttgcttcaac

SEQ ID NO: 26 - DNA, 22 bp, Artificial Sequence
  Synthetic construct, PCR reverse primer for 3′UTR of Mmp9 gene
gactgccaggaagacacttggt

SEQ ID NO: 27 - DNA, 18 bp, Artificial Sequence
  Synthetic construct, PCR forward primer for 3′UTR of Mitf gene
cgagcctgccttgctctg

SEQ ID NO: 28 - DNA, 22 bp, Artificial Sequence
  Synthetic construct, PCR reverse primer for 3′UTR of Mitf gene
gcatcatgctctgggtatgacc

SEQ ID NO: 29 - DNA, 21 bp, Artificial Sequence
  Synthetic construct, PCR forward primer for 3′UTR of Itgav gene
ttcaacagttcctcaaggcct

SEQ ID NO: 30 - DNA, 26 bp, Artificial Sequence
  Synthetic construct, PCR reverse primer for 3′UTR of Itgav gene
catgttgtaaatcaagcagaacactg

SEQ ID NO: 31 - DNA, 21 bp, Artificial Sequence
  Synthetic construct, PCR forward primer for 3′UTR of Ctsk gene
cagccaaatccatcctgctct

SEQ ID NO: 32 - DNA, 23 bp, Artificial Sequence
  Synthetic construct, PCR Reverse primer for 3′UTR of Ctsk gene
aaagtgcaaagaagggaagacaa

SEQ ID NO: 33 - DNA, 22 bp, Artificial Sequence
  Synthetic construct, PCR forward primer for 3′UTR of Mmp14 gene
taccacaaggactttgcctctg

SEQ ID NO: 34 - DNA, 21 bp, Artificial Sequence
  Synthetic construct, PCR reverse primer for of Mmp14 gene
atctagccgaactgccagcac

SEQ ID NO: 35 - DNA, 22 bp, Artificial Sequence
  Synthetic construct, PCR forward primer for 3′UTR of Nfatc1 gene
acattggagcactcagttcagc

SEQ ID NO: 36 - DNA, 23 bp, Artificial Sequence
  Synthetic construct, PCR reverse primer for 3′UTR of Nfatc1 gene
cgtttccttttcaatgacagtcc

SEQ ID NO: 37 - DNA, 22 bp, Artificial Sequence
  Synthetic construct, PCR reverse primer for 3′UTR of Cpe gene
gtagtaagatgcaatgtggctc

SEQ ID NO: 38 - DNA, 23 bp, Artificial Sequence
  Synthetic construct, PCR reverse primer for 3′UTR of Cpe gene
ctttattcatcagaatgcaattc

SEQ ID NO: 39 - DNA, 19 bp, Artificial Sequence
  Synthetic construct, PCR forward primer for 3′UTR of Fosb gene
caaacccgcaaggaacaag

SEQ ID NO: 40 - DNA, 20 bp, Artificial Sequence
  Synthetic construct, PCR reverse primer for 3′UTR of Fosb gene
ggcgacagtgcagaaccaag

SEQ ID NO: 41 - DNA, 23 bp, Artificial Sequence
  Synthetic construct, PCR forward primer for 3′UTR of Traf6 gene
tcagtgaggtcacgagccacttc

SEQ ID NO: 42 - DNA, 22 bp, Artificial Sequence
  Synthetic construct, PCR reverse primer for 3′UTR of Traf6 gene
agctgagtgtccacacaaccgt

SEQ ID NO: 43 - DNA, 19 bp, Artificial Sequence
  Synthetic construct, PCR forward primer for 3′UTR of Oscar gene
caacctgagtggcggagaa

SEQ ID NO: 44 - DNA, 25 bp, Artificial Sequence
  Synthetic construct, PCR reverse primer for 3′UTR of Oscar gene
gcagcagcaagatttattgtgtaga

SEQ ID NO: 45 - DNA, 51 bp, Artificial Sequence
  Synthetic construct, PCR forward primer for 3′UTR of Calcr-141mut
aggtatttttccaatcagactctaatgtagtattgttctatttttgtttac

SEQ ID NO: 46 - DNA, 51 bp, Artificial Sequence
  Synthetic construct, PCR reverse primer for 3′UTR of Calcr-141mut
gtaaacaaaaatagaacaatactacattagagtctgattggaaaaatacct

SEQ ID NO: 47 - DNA, 42 bp, Artificial Sequence
  Synthetic construct, PCR forward primer for 3′UTR of Mift-141mut1
atcatcatttttttctgactatgtattaatttgtaagaatat

SEQ ID NO: 48 - DNA, 42 bp, Artificial Sequence
  Synthetic construct, PCR reverse primer for 3′UTR of Mift-141mut1
atattcttacaaattaatacatagtcagaaaaaaatgatgat

SEQ ID NO: 49 - DNA, 42 bp, Artificial Sequence
  Synthetic construct, PCR forward primer for 3′UTR of Mift-141mut2
gggactggttaagccaagacgctgactatttgctcactgttt

SEQ ID NO: 50 - DNA, 47 bp, Artificial Sequence
  Synthetic construct, PCR reverse primer for 3′UTR of Mift-141mut2
aaacagtgagcaaatagtcagcgtcttggcaaggcttaaccagtccc

SEQ ID NO: 51 - DNA, 20 bp, Artificial Sequence
  Synthetic construct, PCR forward primer for 3′UTR of Mift-219mut
cattgtctgtttttctcttc

SEQ ID NO: 52 - DNA, 48 bp, Artificial Sequence
  Synthetic construct, PCR reerse primer for 3′UTR of Mift-219mut
gaagagaaaaacagacaatgctaagttagaatcggtgaacactatgga

SEQ ID NO: 53 - DNA, 53 bp, Artificial Sequence
  Synthetic construct, PCR forward primer for 3′UTR of Traf6-219mut
tcttctgttgcttgcaaacactataacaccagcccaaacgagtgatttagctc

SEQ ID NO: 54 - DNA, 53 bp, Artificial Sequence
  Synthetic construct, PCR reverse primer for 3′UTR of Traf6-219mut
gagctaaatcactcgtttgggctggtgttatagtgtttgcaagcaacagaaga

SEQ ID NO: 55 - DNA, 39 bp, Artificial Sequence
  Synthetic construct, PCR forward primer for 3′UTR of Mift-133amut
cctccgtttagagtccactatataccaaaattatccacc

SEQ ID NO: 56 - DNA, 38 bp, Artificial Sequence
  Synthetic construct, PCR reverse primer for 3′UTR og Mift-133amut
ggtggataatttggtatatagtggactctaaacggagg

SEQ ID NO: 57 - DNA, 36 bp, Artificial Sequence
  Synthetic construct, PCR forward primer 3′UTR of Mmp14-133amut
agggggcaggaggcgtcgataaaggaaaatgaggac

SEQ ID NO: 58 - DNA, 36 bp, Artificial Sequence
  Synthetic construct, PCR reverse primer for 3′UTR of Mmp14-133amut
gtcctcattttcctttatcgacgcctcctgccccct

SEQ ID NO: 59 - DNA, 18 bp, Artificial Sequence
  Synthetic construct, PCR forward primer for 3′UTR of Calcr-190mut
gaagaattattcagtgaa

SEQ ID NO: 60 - DNA, 46 bp, Artificial Sequence
  Synthetic construct, PCR reverse primer for 3′UTR of Calcr-190mut
ttcactgaataattcttcacaaatcaaaagatcggttcctgggagg

SEQ ID NO: 61 - RNA, 22 nt, Homo sapiens
  misc_feature (1)..(22): miR-133a
uuugguccccuucaaccagcua

SEQ ID NO: 62 - RNA, 23 nt, Homo sapiens
  misc_feature (1)..(23): Traget mRNA-MITF
aaaccaggggaggacgggggaga

SEQ ID NO: 63 - RNA, 22 nt, Homo sapiens
  misc_feature (1)..(22): Target mRNA -MITF
aaaccagugagguauaucuccg

SEQ ID NO: 64 - RNA, 22 nt, Homo sapiens
  misc_feature (1)..(22): miR-190
ugauauguuugauauauuaggu

SEQ ID NO: 65 - RNA, 23 nt, Homo sapiens
  misc_feature (1)..(23): Target mRNA-CALCR
uacuauaccagucgaggacccuc

SEQ ID NO: 66 - RNA, 21 nt, Homo sapiens
  misc_feature (1)..(21): miR-219
ugauuguccaaacgcaauucu

SEQ ID NO: 67 - RNA, 23 nt, Homo sapiens
  misc_feature (1)..(23): Target mRNA-MITF
uacuaacauagccaguugugaua

SEQ ID NO: 68 - RNA, 23 nt, Homo sapiens
  misc_feature (1)..(23): Target mRNA-TRAF6
gacuaacauacacacguagagga

SEQ ID NO: 69 - RNA, 22 nt, Homo sapiens
  misc_feature (1)..(22): miR-141
uaacacugucugguaacgaugu

SEQ ID NO: 70 - RNA, 23 nt, Homo sapiens
  misc_feature (1)..(23): Target mRNA -CALCR site 1
auugugacacuaacuuuuuugua

SEQ ID NO: 71 - RNA, 22 nt, Homo sapiens
  misc_feature (1)..(22): Target mRNA-CALCR site 2
auugugaagaaguguuuacuau

SEQ ID NO: 72 - RNA, 23 nt, Homo sapiens
  misc_feature (1)..(23): Target mRNA MITF site 1
uuugugacuuuuuuuacuacuau

SEQ ID NO: 73 - RNA, 23 nt, Homo sapiens
  misc_feature (1)..(23): Target mRNA-MITF site 2
uuugugacgcagaaccgaauugg

SEQ ID NO: 74 - RNA, 69 nt, Homo sapiens
  misc_feature (1)..(69): miR-33a
cuguggugcauuguaguugcauugcauguucuggugguacccaugcaauguuuccacagugcaucacag

SEQ ID NO: 75 - RNA, 66 nt, Homo sapiens
  misc_feature (1)..(66): miR-378
agggcuccugacuccagguccuguguguuaccuagaaauagcacuggacuuggagucagaaggccu

SEQ ID NO: 76 - RNA, 89 nt, Homo sapiens
  misc_feature (1)..(89): miR-16
gucagcagugccuuagcagcacguaaauauuggcguuaagauucuaaaauuaucuccaguauuaacugugcugcugaaguaagguugac

SEQ ID NO: 77 - RNA, 69 nt, Homo sapiens; identical to SEQ ID NO: 74
  misc_feature (1)..(69): Precusor miR-33a

SEQ ID NO: 78 - RNA, 88 nt, Homo sapiens
  misc_feature (1)..(88): Precusor miR-133a
acaaugcuuugcuagagcugguaaaauggaaccaaaucgccucuucaauggauuugguccccuucaaccagcuguagcuaugcauuga

SEQ ID NO: 79 - RNA, 88 nt, Homo sapiens; identical to SEQ ID NO: 78
  misc_feature (1)..(88): Precusor miR-141

SEQ ID NO: 80 - RNA, 85 nt, Homo sapiens
  misc_feature (1)..(85): Precusor miR-190
ugcaggccucugugugauauguuugauauauuagguuguuauuuaauccaacuauauaucaaacauauuccuacagugucuugcc

SEQ ID NO: 81 - RNA, 110 nt, Homo sapiens
  misc_feature (1)..(110): Precusor miR-219
ccgccccgggccgcggcuccugauuguccaaacgcaauucucgagucuauggcuccggccgagaguugagucuggacgucccgagccgccgcccccaaaccucgagcggg

SEQ ID NO: 82 - RNA, 69 nt, Artificial Sequence; identical to SEQ ID NO: 74
  Synthetic construct, precursor miR-33(a)(2)

SEQ ID NO: 83 - RNA, 88 nt, Artificial Sequence; identical to SEQ ID NO: 78
  Synthetic construct, precursor miR-133(a)(2)

SEQ ID NO: 84 - RNA, 95 nt, Artificial Sequence
  Synthetic construct, precursor miR-141(2)
cggccggcccuggguccaucuuccaguacaguguuggauggucuaauugugaagcuccuaacacugucugguaaagauggcucccggguggguuc

SEQ ID NO: 85 - RNA, 85 nt, Artificial Sequence; identical to SEQ ID NO: 80
  Synthetic construct, precursor miR-190(2)

SEQ ID NO: 86 - RNA, 110 nt, Artificial Sequence; identical to SEQ ID NO: 81
  Synthetic construct, precursor miR-219(2)

(19) United States
(12) Patent Application Publication
(10) Pub. No.: US 2016/0017326 A1
(43) Pub. Date: Jan. 21, 2016

(54) MICRORNAS AS FUNCTIONAL MEDIATORS AND BIOMARKERS OF BONE METASTASIS

(71) Applicant: THE TRUSTEES OF PRINCETON UNIVERSITY, Princeton, NJ (US)

(72) Inventors: Yibin Kang, Princeton, NJ (US); Brian Ell, North Brunswick, NJ (US)

(73) Assignee: The Trustees of Princeton University, Princeton, NJ (US)

(21) Appl. No.: 14/775,288

(22) PCT Filed: Mar. 14, 2014
(86) PCT No.: PCT/US2014/027573

§ 371 Date: Sep. 11, 2015

Related U.S. Application Data
(60) Provisional application No. 61/784,724, filed on Mar. 14, 2013.

(51) Int. Cl. C12N 15/113; G01N 33/574; A61K 39/395; A61N 5/10; A61K 9/51; A61K 47/30; A61K 47/36; C12Q 1/68; A61K 9/127

(52) U.S. Cl. CPC C12N 15/113; C12Q 1/6886; G01N 33/57407; A61K 39/3955; A61K 9/127; A61K 9/51; A61K 47/30; A61K 47/36; A61N 5/10; C12Q 2600/178; C12Q 2600/106; G01N 2333/70525; C12N 2310/141; C12N 2320/31

(57) ABSTRACT

Pharmaceutical compositions that include therapeutic agents including miRNA nucleic acid sequences are provided. Methods of diagnosing and treating a subjects suffering from a bone degenerative diseases including osteolytic bone metastasis are described.

### CROSS REFERENCE TO RELATED APPLICATIONS

This application claims the benefit of U.S. Provisional Application No. 61/784,724, filed Mar. 14, 2013, which is incorporated by reference as if fully set forth.

### GOVERNMENT RIGHTS

This invention was made with government support under Grants No. CA141062 and No. CA134519 awarded by the National Institutes of Health and Grant No. W81XWH-13-1-0425 awarded by the Department of Defense, Army Medical Research & Material Command. The government has certain rights in the invention

The sequence listing electronically filed with this application titled “Sequence Listing,” which was created on Mar. 14, 2014, had a file size of 21,023 bytes, and is incorporated by reference herein as if fully set forth.

### FIELD

The disclosure herein relates to pharmaceutical compositions and methods for the diagnosis, prognosis and treatment of a bone degeneration disease including bone metastasis. Specifically, the disclosure relates to microRNAs associated with osteoclastogenesis and osteolyic bone metastasis, and related nucleic acids.

### BACKGROUND

Osteolytic bone metastasis is a frequent occurrence in late stage breast, lung, thyroid, bladder, and many other types of cancer, leading to pathological fractures, pain, and hypercalcemia (Weilbaecher et al., 2011). The development of bone lesions depends upon the orchestrated interactions between tumor cells and functional cells within the bone, namely osteoblasts and osteoclasts (Ell and Kang, 2012; Weilbaecher et al., 2011).

The bone resorbing osteoclasts play an important role in physiological bone remodeling, while aberrant osteoclast activity can lead to pathological conditions including Paget's disease and lytic bone metastasis (Boyle et al., 2003; Teitelbaum and Ross, 2003; Weilbaecher et al., 2011). Osteoclast differentiation is canonically dependent on two essential molecules, macrophage colony-stimulating factor (M-CSF) and receptor activator of NF-κB ligand (RANKL), although a number of RANKL independent pathways have been described ((Boyle et al., 2003; Teitelbaum and Ross, 2003; Hemingway et al., 2011). Aberrant expression of these signaling molecules by bone-metastatic cancer cells has been shown to recruit pre-osteoclasts to the site of osteolytic metastasis and induce their differentiation, leading to degradation of the bone and the subsequent release of bone matrix-embedded tumor-promoting growth factors such as TGFβ (Ell and Kang, 2012; Korpal et al., 2009; Weilbaecher et al., 2011). The role of osteoclasts in bone metastasis is further underscored by the efficacy of treatments targeting osteoclast differentiation and activity (Clezardin, 2011). MicroRNAs have been recognized as players during osteoclast differentiation, as genetic or siRNA-mediated ablation of factors important for biogenesis of miRNAs, including Dicer1, Dgcr8 and Ago2, blocked osteoclast differentiation (Mizoguchi et al., 2010; Sugatani and Hruska, 2009). Consistent up- and down-regulated miRNAs were observed during osteoclast differentiation in bth pathological and physiological conditions. Additionally, ectopic expression of miR-155 or repression of miR-21 inhibit osteoclast differentiation, while conflicting functions of miR-223 in osteoclastogenesis have also been reported (Mann et al., 2010; Mizoguchi et al., 2010; Zhang et al., 2012; Sugatani et al., 2011; Sugatani and Hruska, 2007; Sugatani and Hruska, 2009).

Current treatments targeting osteoclasts, such as bisphosphonoates and denosumabs are able to limit the pathology associated with bone metastasis, although without significant improvement to the survival of patients.

### SUMMARY

In an aspect, the invention relates to a pharmaceutical composition that includes a therapeutic agent. The therapeutic agent includes a first nucleic acid with at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86). The pharmaceutical composition also includes at least one additional therapeutic agent.

In an aspect, the invention relates to a method of treating a subject suffering from a bone degeneration disease. The method includes administering any one of the pharmaceutical compositions described herein to the subject.

In an aspect, the invention relates to a method of treating a subject suffering from a bone degenerative disease. The method includes obtaining a test sample from the subject. The method also includes determining an expression level of at least one osteoclast differentiation marker in the test sample. The at least one osteoclast differentiation marker is one or more of miRNAs or sICAM1. The method includes comparing the expression level of the at least one osteoclast differentiation marker in the test sample to the expression level of the at least one osteoclast differentiation marker in a reference sample. An increase in the expression level of the at least one osteoclast differentiation marker in the test sample compared to the reference sample indicates the subject will be a positive responder. A finding of a decrease in the expression level of the at least one osteoclast differentiation marker in the test sample compared to the reference sample indicates the subject will be a negative responder. The method further includes one or more of recommending or conducting at least one of: treating the subject with any one of the pharmaceutical compositions herein if the subject is determined to be a positive responder, and treating the subject with one or more of radiation therapy or immunotherapy if the subject is determined to be a negative responder.

In an aspect, the invention relates to a method for diagnosing whether a subject has bone metastasis. The method includes obtaining a test sample from a subject afflicted with cancer. The method includes determining an expression level of at least one osteoclast differentiation marker in the test sample. The method includes comparing the expression level of the at least one osteoclast differentiation marker in the test sample to the expression level of the corresponding osteoclast differentiation marker in a reference sample. The method also includes diagnosing the subject as having bone metastasis if the expression level of the at least one osteoclast differentiation marker in the test sample is elevated compared to the corresponding osteoclast differentiation marker in the reference sample.

### BRIEF DESCRIPTION OF THE DRAWINGS

The following detailed description of embodiments of the present invention will be better understood when read in conjunction with the appended drawings. For the purpose of illustration, there are shown in the drawings embodiments which are presently preferred. It is understood, however, that the invention is not limited to the precise arrangements and instrumentalities shown. In the drawings:

FIGS. 1A-1F illustrate RANKL and conditioned media of highly bone metastatic cancer cell lines induce similar changes in miRNA expression in osteoclasts.

FIG. 1A illustrates representative images of RAW264.7 cells stained for TRAP after treatment for 6 days with conditioned media from indicated cell lines, or conditioned media with 20 ng/ml RANKL (RANKL^{Low}), or 50 ng/ml RANKL (RANKL^{High}).

FIG. 1B illustrates quantification of TRAP⁺ osteoclasts from experiments in FIG. 1A.

FIG. 1C illustrates quantification of nuclei in TRAP⁺ osteoclasts from experiments in FIG. 1A.

FIG. 1D illustrates a heat map depicting miRNA microarray expression profiling in RAW264.7 cells treated with conditioned media from 4T1.2 or TSU-Pr1-B2 cells as compared to corresponding weakly metastatic cells (4T1 and TSU-Pr1), or 50 ng/ml RANKL as compared to non-treatment control.

FIG. 1E illustrates a correlation of miRNA expression changes in the indicated treatment pairs as in FIG. 1D.

FIG. 1F illustrates qRT-PCR analysis of selected miRNAs in RAW264.7 or primary bone marrow derived osteoclasts after the indicated length of treatment with 50 ng/ml RANKL.

FIGS. 2A-2K illustrate osteoclast differentiation induced by RANKL and tumor conditioned media.

FIG. 2A illustrates representative images of TRAP stained RAW264.7 cells treated with the indicated concentration of RANKL for 6 days.

FIG. 2B illustrates quantification of TRAP⁺ osteoclasts and nuclei per osteoclast from experiments in FIG. 2A.

FIG. 2C illustrates representative images of TRAP stained culture of primary bone marrow cells.

FIG. 2D illustrates representative images of TRAP stained MOCP-5 cells.

FIG. 2E illustrates quantification of TRAP⁺ osteoclasts treated with indicated conditions from experiment in FIG. 2D.

FIG. 2F illustrates quantification of nuclei in TRAP⁺ osteoclasts as in FIG. 2E.

FIGS. 2G and 2H illustrate qRT-PCR mRNA expression levels of osteoclast genes in RAW264.7 cells under indicated treatments.

FIG. 2I illustrates representative images of TRAP stained human osteoclasts.

FIGS. 2J and 2K illustrate qRT-PCR miRNA (FIG. 2J) and mRNA expression levels (FIG. 2K) in osteoclasts from FIG. 2I

FIG. 3A-3D illustrate ectopic expression of miRNAs downregulated during osteoclastogenesis inhibits osteoclast differentiation.

FIG. 3A illustrates quantification and representative images of TRAP⁺ osteoclasts in RAW264.7 cells transfected with indicated miRNAs.

FIG. 3B illustrates quantification and representative images of bone resorption by RAW264.7 cells after treatment in conditions as in FIG. 3A.

FIG. 3C illustrates quantification of TRAP⁺ osteoclasts from RAW264.7 treated cells transfected with 1 pM indicated miRNAs.

FIG. 3D illustrates quantification of relative bone resorption in RAW264.7 cells transfected with 1 pM indicated miRNA or 20 nM ZOMETA™.

FIGS. 4A-4H illustrate inhibition of osteoclast differentiation by ectopic expression of miRNAs downregulated during osteoclastogenesis.

FIG. 4A illustrates quantification of TRAP⁺ osteoclasts in primary bone marrow derived osteoclasts transfected with 1 pM of indicated miRNA oligonucleotide.

FIG. 4B illustrates RT-PCR mRNA expression levels of indicated genes in RAW264.7 cells transfected with miRNAs, followed by treatment with 50 ng/ml RANKL.

FIG. 4C illustrates representative images of bone resorption in RAW264.7 cells transfected with indicated miRNAs or treated with 20 nM ZOMETA™.

FIG. 4D illustrates representative images of TRAP⁺ osteoclasts from MOCP-5 cells transfected with indicated miRNAs, followed by treatment with 50 ng/mL RANKL.

FIG. 4E illustrates quantification of TRAP⁺ osteoclasts from experiment in FIG. 4D.

FIG. 4F illustrates representative images of RAW264.7 cells treated with indicated miRNAs and grown on Jagged1 coated plates, followed by treatment with 50 ng/mL RANKL.

FIG. 4G illustrates quantification of TRAP⁺ osteoclasts from experiment in FIG. 4F.

FIG. 4H illustrates quantification of nuclei per osteoclast from experiment in FIG. 4F.

FIGS. 5A-5E illustrate validation of candidate miRNA genes.

FIGS. 5A-5D illustrate normalized activity of luciferase reporter containing the 3′ UTR of candidate genes upon co-transfection with indicated miRNAs, relative to transfection with negative control miRNA. *p<0.05.

FIG. 5E schematically illustrates miRNA targets that are involved in osteoclast differentiation or function.

FIG. 5F illustrates Western blot analysis for Pu.1, Nfatc1, or Ctsk in RAW264.7 cells after transient transfection with indicated miRNA and treatment with 50 ng/ml RANKL.

FIG. 6A-6F illustrate micro-computed tomography and histological analysis showing alterations in bone homeostasis after treatment with miRNAs downregulated in osteoclastogenesis.

FIG. 6A illustrates representative X-ray, μCT and histological images for TRAP, H&E, Von Kossa, or osteocalcin staining of bones from mice treated with indicated miRNAs.

FIG. 6B illustrates quantification of bone volume relative to total volume from representative μCT scans in FIG. 6A.

FIG. 6C illustrates quantification of trabecular thickness from representative μCT scans of mice treated with miRNAs as in FIG. 6B.

FIG. 6D illustrates quantification of cortical bone thickness from representative μCT scans as in FIG. 6B.

FIG. 6E illustrates quantification of TRAP⁺ osteoclasts from decalcificed histological sections of hindlinbs from mice in FIG. 6A.

FIG. 6F illustrates quantification of osteocalsin-positive osteoblasts from bone sections from mice in FIG. 6A.

FIGS. 7A-7I illustrate conditioned media of bone-tropic sublines of MDA-MD-231 induces osteoclast differentiation.

FIG. 7A illustrates representative images of RAW264.7 cells treated with 50 ng/ml RANKL or indicated CM.

FIG. 7B illustrates quantification of osteoclast differentiation from RAW264.7 cells in experiment in FIG. 7A.

FIG. 7C illustrates qRT-PCR mRNA expression levels of indicated genes in RAW264.7 cells treated with CM from indicated cell lines.

FIG. 7D illustrates heat map depicting qRT-PCR miRNA expression profiling in RAW264.7 cells after treatment with CM from the indicated cell lines relative to the treatment with CM from the MDA-MB-231 parental cell line.

FIG. 7E illustrates representative images of MOCP-5 cells treated with indicated CM.

FIGS. 7F and 7G illustrate quantification of osteoclast number and nuclei per osteoclast after MOCP-5 cells were treated with indicated CM as in FIG. 7C.

FIG. 7H illustrates representative BLI images of mice after inoculation with SCP28 cells and treatment with indicated miRNAs or 100 μg/kg Zometa.

FIG. 7I illustrates normalized bone metastasis BLI signals from mice in FIG. 6H.

FIGS. 8A-8F illustrate systemic treatment with miRNAs downregulated in osteoclastogenesis inhibits bone metastasis.

FIG. 8A illustrates BLI, X-ray, Osteocalcin, H&E, and TRAP images from mice inoculated with SCP28 breast cancer cells and treated with indicated miRNAs. BLI images show three representative mice from each experimental group.

FIG. 8B illustrates normalized bone metastasis BLI signals from mice in FIG. 8A. n=10.

FIG. 8C illustrates qRT-PCR miRNA expression levels from serum samples of mice taken at indicated times after injection with miRNAs.

FIG. 8D illustrates quantification of osteolytic lesion area in hindlimbs from indicated experimental group.

FIG. 8E illustrates quantification of TRAP⁺ osteoclasts from histology in experiment from FIG. 8A.

FIG. 8F illustrates quantification of osteocalcin⁺ osteoblasts from histology in experiment from FIG. 8A.

FIGS. 9A-9D illustrate serum levels of miRNAs upregulated during osteoclastogenesis correlate with bone metastasis.

FIG. 9 A illustrates BLI and qRT-PCR expression analysis of miRNAs in mice after intravenous inoculation with SCP2 breast cancer cells.

FIG. 9B illustrates BLI and qRT-PCR expression analysis of miRNAs in mice 28 days after intravenous inoculation with TSU-Pr1 or TSU-Pr 1-B2 bladder cancer cells.

FIG. 9C illustrates qRT-PCR miRNA expression analysis of matched micro-dissected primary breast tumor (Primary) or bone metastasis (BM) samples. n=12. p values based on Mann-Whitney test.

FIG. 9D illustrates qRT-PCR miRNA expression analysis of serum samples from healthy donors (HD) or breast cancer patients with bone metastasis (BM).

FIGS. 10A-10I illustrate sICAM1 mediated osteoclast differentiation is 132 integrin dependent.

FIG. 10A illustrates RANKL protein level in indicated CM samples as determined by ELISA.

FIG. 10B illustrates representative images of RAW264.7 cells treated with RANKL or the indicated CM±200 ng/mL OPG as in FIG. 10A.

FIG. 10C illustrates qRT-PCR mRNA expression levels of indicated genes in RAW264.7 cells treated with 30 ng/mL RANKL±sICAM1 for indicated length of time.

FIG. 10D illustrates representative images of RAW264.7 cells treated with 4 ng/ml RANKL plus indicated concentration of sICAM1 for 6 days as in FIG. 10F.

FIG. 10E illustrates representative images of RAW264.6 cells treated with 30 ng/ml RANKL or SCP28 CM±50 μg/ml ICAM1 antibody.

FIG. 10F illustrates representative images and quantification of RAW264.7 cells treated with SCP28 CM±50 ng sICAM1 antibody±200 ng/mL OPG.

FIG. 10G illustrates quantification and representative images of bone resorption in RAW264.7 cells treated with SCP28 CM±50 ng/ml sICAM1 antibody.

FIG. 10H illustrates RAW264.7 cell chemotaxis to indicated treatment of sICAM1 or SCP28 CM, with or without treatment of 50 ng/ml sICAM1 antibody.

FIG. 10I illustrates representative images of RAW264.7 cells treated with RANKL or SCP28 CM±20 ng ICAM1 antibody or 20 ng 132 integrin antibody. Scale bar, 200 μm.

FIGS. 11A-11G illustrate soluble ICAM1 in tumor conditioned media synergizes with RANKL to promote osteoclast differentiation.

FIG. 11A illustrates quantification of RAW264.7 cells treated with 30 ng/ml RANKL or conditioned media±200 ng/ml OPG.

FIG. 11B illustrates heat map from cytokine array depicting relative protein expression in conditioned media from indicated cell lines as compared to their weakly metastatic counterparts (4T1 vs 4T1.2, TSU-Pr1 vs TSU-PR1-B2, and MDA-231 vs SCP28, respectively).

FIG. 11C illustrates quantification of TRAP⁺ osteoclasts from RAW264.7 cells treated with 50 ng/ml RANKL±50 ng/ml sICAM1 for indicated length of time.

FIG. 11D illustrates quantification of TRAP⁺ osteoclasts from RAW264.7 cells treated with 10 ng/ml RANKL, 50 ng/ml sICAM1, or both for 6 days.

FIG. 11E illustrates qRT-PCR analysis of selected miRNAs in RAW264.7 cells after indicated length of treatment with 10 ng/ml RANKL or/and 50 ng/ml sICAM1.

FIG. 11F illustrates quantification of osteoclasts/field and nuclei/osteoclast in RAW264.7 cells treated with 4 ng/ml RANKL plus indicated concentration of sICAM1 for 6 days.

FIG. 11G illustrates quantification of TRAP⁺ osteoclasts from RAW264.7 cells treated with 30 ng/ml RANKL or SCP28 conditioned media followed by treatment with indicated concentration of ICAM1 antibody (in μg/mL).

FIGS. 12A to 12H illustrate that sICAM1 promotes osteoclastogenesis by activating NFκB signaling, and sICAM1 levels in patient serum samples are correlated with miR-16 and miR-378 levels, and associated with bone metastasis.

FIG. 12 A illustrates quantification of TRAP⁺ osteoclasts from RAW264.7 cells treated with RANKL or SCP28 conditioned media±20 μg ICAM1 antibody or 20 μg β2 integrin antibody.

FIG. 12B illustrates expression of αL, αM, and β2 integrin subunits on RAW264.7 by IF (left) and flow cytometry (right).

FIG. 12C illustrates normalized activity of NFκB luciferase reporter in RAW264.7 cells after indicated treatment with TNFα, RANKL, or sICAM1 for indicated length of time.

FIG. 12D illustrates Western blot analysis of IκB in RAW264.7 cells after treatment with TNFα, RANKL, or sICAM1 for indicated length of time.

FIG. 12E illustrates Western blot analysis of IκB in RAW264.7 cells after treatment with indicated concentration of RANKL or SCP28 CM±20 ng β2 antibody or 200 ng/ml OPG for 2 hours.

FIG. 12F illustrates relative qRT-PCR miRNA expression level in RAW264.7 cells treated with 50 ng/ml sICAM1, with or without 20 μM IKK inhibitor PS1145 for 6 days, after normalization to the expression level without sICAM1 treatment.

FIG. 12G illustrates ELISA quantification of sICAM1 expression levels in serum samples collected from healthy female donors (HD, n=41), disease-free breast cancer patients (DFP, n=16), or breast cancer patients with bone metastasis (BM, n=38).

FIG. 12H illustrates correlation of qRT-PCR miR-16 or miR-378 expression levels with ELISA sICAM1 protein expression levels in serum samples from healthy donors or bone metastasis patients from FIG. 12G.

FIGS. 13A-13G illustrate functional and clinical significance of sICAM1 and osteoclast miRNAs in bone metastasis.

FIG. 13A illustrates Western blot analysis of phosphorylated-p65 in RAW264.7 cells after the indicated treatment.

FIG. 13B illustrates Western blot analysis of IκB or p-p65 in RAW264.7 cells after the indicated treatment.

FIG. 13C illustrates Western blot analysis of IκB in HeLa cells after transient transfection with indicated miRNA, followed by 30 minute treatment with TNFα, RANKL, or sICAM-1.

FIG. 13D illustrates normalized activity of NFκB luciferase reporter in HeLa cells after treatments as in FIG. 13C.

FIG. 13E illustrates ROC curves for the diagnosis of breast cancer bone metastasis using miR-16, sICAM1, NTX, or combinations of markers.

FIG. 13F illustrates sensitivity and specificity values for diagnosis of bone metastasis relative to patients with no evidence of disease (NED) or healthy donors (HD) for indicated markers.

### DETAILED DESCRIPTION OF EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

In an embodiment, a pharmaceutical composition is provided. The pharmaceutical composition may include 1) a therapeutic agent and 2) at least one additional therapeutic agent. The therapeutic agent may comprise, consist essentially of, or consist of include a first nucleic acid with at least 70, 72, 75, 80, 85, 90, 91, 92, 93, 94, 95, 96, 97, 98, 99, or 100% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), and miR-141 (SEQ ID NO: 69).

As used herein, the term “micro RNA” or “miRNA” refers to a non-coding RNA that is capable of repressing gene expression through complementary binding of the sequence of target mRNAs. The miRNA may be a mature miRNA of 19 to 24 nucleotides in length. The miRNA may be a precursor miRNA of 70 to 200 nucleotides in length. The first nucleic acid may include a sequence with at least 70, 72, 75, 80, 85, 90, 91, 92, 93, 94, 95, 96, 97, 98, 99, or 100% identity to a sequence selected from the group consisting of: precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86). The mature miRNA may be processed from the precursor miRNA by a processing enzyme. The processing enzyme may but is not limited to be RNAse III, Dicer, or Argonaut. The mature miRNA may be processed by processing enzymes that occur naturally in cells or cell lysates. The mature miRNA may be processed using isolated processing enzymes. The miRNA may be synthesized chemically. The miRNAs may be regulators of cancer metastasis. The miRNAs may be stromal miRNAs. The stromal miRNAs may be mediators and biomarkers of cancer metastasis. The stromal miRNAs may be regulators of osteoclastogenesis. The miRNA that regulate osteoclastogenesis may be regulators of osteolytic bone metastasis.

The miRNAs may activate osteoclasts during breast cancer bone metastasis. The miRNA that activate osteoclasts may be but is not limited to miR-141, miR-219, miR-190, miR-33a, or miR-133a. Ectopic expression of these miRNAs may inhibit osteoclast maturation in multiple models of differentiation, including canonical differentiation using RANKL or conditioned media from human and murine bone-metastatic cancer cell lines. In particular, miR-133a and miR-144 a directly target the osteaoclast transcription factor MITF, while miR-141 and miR-190 may target CALCR.

As used herein, the term “bone metastasis” refers to metastastic bone disease, or cancer metastases that results from primary tumor invasion to bone. Invasion of the bone compartment by cancer cells causes imbalance between osteoclasts and osteoblasts, and leads to osteolytic bone metastasis. Bone metastasis may be present in multiple cancers including the vast majority of late-stage breast cancer patients. Bone metastasis may result in severe bone loss, debilitating fractures, and other life-threatening complications.

In an embodiment, the at least one additional therapeutic agent may comprise, consist essentially of, or consist of a second nucleic acid with least 70, 72, 75, 80, 85, 90, 91, 92, 93, 94, 95, 96, 97, 98, 99, or 100% identity to a sequence is selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86).

Determining percent identity of two nucleic acid sequences or two amino acid sequences may include aligning and comparing the nucleotides or amino acid residues at corresponding positions in the two sequences. If all positions in two sequences are occupied by identical amino acid residues or nucleotides then the sequences are said to be 100% identical. Percent identity may be measured by the Smith Waterman algorithm (Smith T F, Waterman M S 1981 “Identification of Common Molecular Subsequences,” J Mol Biol 147: 195-197, which is incorporated herein by reference as if fully set forth). Percent identity refers to the percent measured along the length of a reference sequence.

In an embodiment, the at least one additional therapeutic agent may be but is not limited to a bone metastasis therapeutic agent, a breast cancer therapeutic agent, or an anti-estrogen therapeutic agent.

The bone metastasis therapeutic agent may be but is not limited to bisphosphonates, zoledronic acid (ZOMETA™), alendronate (FSAMAX™), ibandronate (BONIVA™), risedronate (ATELVIA™), pamidronate (AREDIA™), RANKL antibody (denosumab; XGEVA™), or sclerostin antibody (romosozumab).

The breast cancer therapeutic agent may be a drug used as a chemotherapy for breast cancer. The breast cancer therapeutic agent may be but is not limited to methotrexate (ABITREXATE™, FOLEX™), paclitaxel, or paclitaxel albumin-stabilized nanoparticle formulation (ABRAXANE™), doxorubicin or doxorubicin hydrochloride (ADRIAMYCIN PFS™, ADRIAMYCIN RDF™), fluorouracil or 5-fluorouracil (ADRUCIL™, EFUDEX™, FLUOROPLEX™), everolimus (AFINITOR™), anastrozole (ARIMIDEX™), capecitabine (XELODA™), cyclophosphamide (NEOSAR™), docetaxel (TAXOTERE™), epirubicin hydrochloride (ELLENCE™), exemestane (AROMASIN™), toremifene (FARESTON™), fulvestrant (FASLODEX™), letrozole (FEMARA™), gemcitabine hydrochloride (GEMZAR™), goserelin acetate (ZOLADEX™), ixabepilone (IXEMPRA™), megestrol acetate (MEGACE™), and lapatinib ditosylate (TYKERB™).

The breast cancer therapeutic agent may be a targeted therapeutic agent for breast cancer. The targeted therapeutic agent for breast cancer may be but is not limited to ado-trastuzumab emtansine (KADCYLA™), trastuzumab (HERCEPTIN™), lapatinib ditosylate (TYKERB™), or pertuzumab (PERJETA™).

The anti-estrogen therapeutic agent may be but is not limited to exemestane (AROMASIN™), or tamoxifen citrate (NOVALDEX™)

The at least one additional therapeutic agent may be selected from the group consisting of: bisphosphonates, alendronate, ibandronate, risedronate, pamidronate, a RANKL antibody, a sclerostin antibody, methotrexate (ABITREXATE™, FOLEX™), paclitaxel, or paclitaxel albumin-stabilized nanoparticle formulation (ABRAXANE™), doxorubicin or doxorubicin hydrochloride (ADRIAMYCIN PFS™, ADRIAMYCIN RDF™), fluorouracil or 5-fluorouracil (ANDRUCIL™, FLUOROPLEX™, EFUDEX™), AFINITOR™ (everolimus), anastrozole (ARIMIDEX™), docetaxel (TAXOTERE™), epirubicin hydrochloride (ELLENCE™), toremifene (FARESTON™), fulvestrant (FASLODEX™), letrozole (FEMARA™), gemcitabine hydrochloride (GEMZAR™), ixabepilone (IXEMPRA™), megestrol acetate (MEGACE™), cyclophosphamide (NEOSAR™), docetaxel (TAXOTERE™), toremifene (FARESTON™), lapatinib or lapatinib ditosylate (TYKERB™), capecitabine (XELODA™), goserelin acetate (ZOLADEX™) exemestane (AROMASIN™), tamoxifen (NOLVADEX™), zoledronic acid (ZOMETA™), trastuzumab (HERCEPTINE™), ado-trastuzumab emtansine (KADCYLA™), and pertuzumab (PERJETA™).

In an embodiment, the at least one additional therapeutic agent may include an antibody. The antibody may be a polyclonal antibody, an intact monoclonal antibody, an antibody fragment, which may be but is not limited to Fab, Fab′, F(ab′)2, an Fv fragment, a single chain Fv (scFv) mutant, a chimeric antibody or a multispecific antibody. A multispecific antibody may be a bispecific antibody generated from at least two intact antibodies. The antibody may be a humanized antibody or a human antibody. The antibody may be a fusion protein comprising an antigen determination portion of an antibody. The antibody may be a fragment of an antibody comprising an antigen recognition site. The antibody may be selected from any of the five major classes of immunoglobulins: IgA, IgD, IgE, IgG, and IgM. The antibodies may be selected from subclasses or isotypes thereof. The antibodies may be selected from the subclass of IgG1, IgG2, IgG3, IgG4, IgA1 or IgA2. The antibody may be an antibody that is based on the identity of its heavy-chain constant domain referred to as alpha, delta, epsilon, gamma, and mu. The antibody may be a naked antibody or an antibody conjugated to other molecules. The antibody may be an antibody conjugated to, for example, toxins or radioisotopes. The antibody may be a humanized or a chimeric antibody. To study in vivo bone metastases in mouse and rat models the humanized antibody may be replaced by mouse- or rat-monoclonal antibodies.

The antibody may have an ability to recognize and specifically bind to a target. The target may be but is not limited to a protein, a polypeptide, a peptide, a carbohydrate, a polynucleotide, a lipid, or combinations of at least two of the foregoing through at least one antigen recognition site within the variable region of the antibody. The antibody may be specific to the Intercellular Adhesion Molecule-1 (ICAM-1). The antibody may be specific to a soluble Intercellular Adhesion Molecule-1 (sICAM1). The antibody may bind sICAM1. The antibody may specifically bind an sICAM1 receptor. The sICAM1 receptor may be a β2 integrin. The β2 integrin may be one of αLβ2 integrin and αMβ2 integrin. The antibody may be a monoclonal antibody.

In an embodiment, the pharmaceutical composition herein may include a pharmaceutically acceptable carrier. The pharmaceutically acceptable carrier may be at least one substance selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.) and phosphate buffered saline (PBS).

In an embodiment, the pharmaceutical composition may be associated with a nanocarrier. The nanocarrier may be but is not limited to lipid nanoparticles, liposomes, polymer particles, ligands, or polydexstrins. The nanocarrier may be lipid nanoparticles. The pharmaceutical composition may be encapsulated within lipid nanoparticles.

An embodiment provides a method of treating a subject suffering from a bone degeneration disease. The method may include administering any pharmaceutical composition herein to the subject. The bone degeneration disease may be but is not limited to bone metastasis, osteoporosis, or Paget disease. The bone degeneration disease may be bone metastasis.

The subject may be a patient. As used herein, the term “patient” refers to a human. The patient may be a human with a symptom or symptoms of the osteolytic bone disease. The patient may need treatment for the osteolytic bone disease in a clinical setting. The symptoms of the disease or condition may change as a result of a treatment, or spontaneous remission, or development of further symptoms with the progression of the disease. The term “patient” may also refer to non-human organism. The patient may be a mammal, a laboratory animal, a farm animal, or a zoo animal. The patient may be a rodent, a mouse, a rat, a guinea pig, a hamster, a horse, a rabbit, a goat, or a cow.

The pharmaceutical composition administered to the subject may be any one of the pharmaceutical composition described herein. The pharmaceutical composition may include a therapeutic agent that includes a first nucleic acid with at least 70, 72, 75, 80, 85, 90, 91, 92, 93, 94, 95, 96, 97, 98, 99, or 100% identity to a sequence selected from the group consisting of: miR-33a (SEQ ID NO; 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86). The subject may be subsequently treated with the pharmaceutical composition that includes at least one additional therapeutic agent as described herein.

In an embodiment, the method provides the pharmaceutical composition in association with a nanocarrier. The nanocarrier may be but is not limited to lipid nanoparticles, liposomes, polymer particles, ligands or polydexstrins. The pharmaceutical composition described herein may be encapsulated inside lipid nanoparticles. The method of preparing lipid nanoparticles is described in U.S. patent application Ser. No. 13/639,628 filed Apr. 7, 2011 as PCT patent application PCT/US11/31540, both of which are incorporated herein by reference as if fully set forth. A method herein may include producing lipid nanoparticles that encapsule the therapeutic agents herein by the following steps: providing one or more aqueous solutions in one or more reservoirs; providing one or more organic solutions in one or more reservoirs, wherein one or more of the organic solutions contains a lipid and wherein one or more of the aqueous solutions and/or one or more of the organic solutions includes therapeutic products; mixing the one or more aqueous solutions with the one or more organic solutions in a first mixing region, wherein the first mixing region is a Multi-Inlet Vortex Mixer (MIVM), wherein the one or more aqueous solutions and the one or more organic solutions are introduced tangentially into a mixing chamber within the MIVM so as to substantially instantaneously produce a lipid nanoparticle solution containing lipid nanoparticles encapsulating therapeutic agents.

The method may include administering the pharmaceutical composition to a subject by any suitable route. The route of administration may be any one or more route including but not limited to: intramuscular injection, subcutaneous injection, intravenous injection, intradermal injection, intranasal injection, inhalation, oral administration, sublingual administration, buccal administration, or topical administration.

The subject may be treated with the pharmaceutical composition until the bone metastasis growth is inhibited. The subject may be treated until bone metastasis is cured.

The responsiveness of the subject to the treatment may be assessed by any suitable method. The responsiveness of the subject to the treatment may be determined by detection of the osteoclast differentiation marker genes in the test sample collected from the subject having a bone degenerative disease and by comparing results to the control samples. The control samples may be collected from a subject before treatment. The control sample may be collected from a healthy individual.

The method may include assessing inhibition of bone metastasis growth in the subject before and after treatment. The assessing may be performed by any known method. The method may include collecting samples from a subject diagnosed with a cancer. Samples may include blood samples, serum or cells. The method may include Bone Resorption Assay, Histomorphometric Analysis and Immunohistochemical Staining, X-ray Analysis and Quantification described in Example 1 herein. The method may include measuring a rate of bone metastasis. The measuring of rate of bone metastasis may include whole-body bone scans of a patient. The whole-body scan may be performed before and after treatment of the patient with a pharmaceutical composition described herein. Bone lesions may be quantified and the regional distribution of metastases may be assessed using statistical methods. Systemic treatment with the pharmaceutical composition that includes one or more miRNAs may result in a decrease in osteoclast number and a subsequent increase in bone density and trabecular area as measured by microCT scans. The subject having breast cancer followed by systemic treatment with the pharmaceutical compositions described herein may demonstrate a decrease in tumor burden and bone lesion. The pharmaceutical composition may include a therapeutic agent that includes at least one of the first nucleic acid or the second nucleic acid, each of which has a sequence selected from the group consisting of miR-33a, miR-133a, miR-190, miR-141, and miR-219. The first nucleic acid or the second nucleic acid each of which has a sequence selected from the group consisting of miR-141, miR-219, miR-190, miR-33a, and miR-133a may inhibit osteoclast differentiation and reduce growth of bone metastasis

A separate subset of miRNAs, including miR-16 and miR-378, may be sensitive and non-invasive biomarkers for early detection of bone metastasis, as well as for monitoring therapeutic response. Elevated miRNA levels in the serum may be used to indicate the development or progression of osteolytic bone metastasis. These miRNAs have may be markers for bone metastasis onset, progression, and response to treatment. An increase in concentration of one or both of miR-16 and miR-378 in serum samples taken from the subject with bone metastasis, relative to healthy controls may indicate progression of the disease. A healthy control may be serum samples taken from a healthy individual.

In an embodiment, the method may include assessing inhibition of bone metastasis by determining expression of at least one osteoclast differentiation marker gene in the subject after treatment. The expression level of the at least one osteoclast differentiation marker may be determined by any method. The method may be but is not limited to a quantitative reverse transcription polymerase chain reaction (RT-PCR), a microarray, Nothern blot analysis, or mass spectrometry.

For mRNA analysis, mRNA may be isolated from any sample described herein using standard methods. The isolated mRNA may be transcribed and amplified by RT-PCR, using oligonucleotide primers specific for a taget gene to create a cDNA from the mRNA. Conditions for primer annealing may be selected to ensure specific reverse transcription and amplification of the target gene. The target gene may be one or more osteoclast differentiation marker genes. The one or more osteoclast differentiation marker gene may be selected from the group consisting of: Mitf, Traf6, Mmp14, Calcr, Cpr, Mmp9, Itgav, Oscar, Nfatc1, Ctsk, Calcr site1, Calcr site2, Mitf site1, and Mitf site 2. As used herein, Mitf refers to the osteosclast marker gene that encodes MITF a basic-helix-loop-helix-zipper transcription factor participates in osteoclast development (Hodgkinson et al., 1993; Hershey and Fisher, 2004; Sharma et al., 2007; Weilbaecher et al., 2001). Traf6 encodes a member of the tumor necrosis factor receptor (TNFR)-associated factor family of cytokine receptor adaptor proteins that play a role in signaling transduction of the RANKL pathway (Lomaga et al., 1999). Calcr encodes calcitonin receptor (CALCR), a cell-surface receptor capable of influencing osteoclast-mediated bone resorption in vitro and in vivo (Davey et al., 2008). Mmp14 knockout mice feature severe skeletal defects, including osteopenia and skeletal dysplasia (Holmbeck et al., 1999). Mmp14 was implicated in osteoclast fusion during maturation, and Mmp14-null osteoclasts had decreased activity in vitro (Gonzalo et al., 2010). Itgav encodes Integrin, alpha V, a member of the integrin superfamily. Oscar encodes the osteoclast-associated, immunoglobulin-like receptor, a member of the leukocyte receptor complex protein family that plays role in the regulation of innate and adaptive response. Nfatc1 encodes NFATC1, a transcription factor that regulates T-cell development, osteoclastogenesis, and macrophage function. Ctsk encodes cathepsin K, a proteinase secreted by osteoclasts that degrades bone.

The presence of the amplification products specific to one or more osteoclast differentiation marker genes may indicate progression of the bone metastasis. The lack of the amplification products specific to one or more osteoclast differentiation marker genes may indicate supression of these genes in the patient resulted from treatment with a pharmaceutical composition, and inhibition of the bone metastasis growth. The cDNA of the target genes may be used in a quantitative PCR assay and may indicate a number of copies of mRNA specific to a target gene. Quantitative PCR may be used identify whether treatment resulted in decrease of expression level of one or more osteoclast differentiation markers. The decrease of one more of the osteoclasts differentiation marker genes may indicate that the bone metastasisbone metastasis growth is inhibited. The method may further include terminating treatment if the bone metastasis growth is inhibited.

The treatment may be terminated if the bone metastasis growth is reduced. The treatment may be terminated if the metastatic lesion growth is limited. The subject may be unresponsive to the treatment with the therapeutic composition and may require additional treatments. In this case, the method may include one or more additional therapeutic treatments. The method may include treating the subject with at least one method selected from the group that consists of: adjuvant therapy, chemotherapy, immune therapy, radiation therapy, gene therapy, surgery, use of prosthetics, vaccination, use of hormones, cytokines, vitamins, chemokines, antibiotic therapy, and transplantation. The method may further include treating the subject with radiation therapy. The method may further include treating the subject with immune therapy. The method may include treating the subject with a combination of radiation therapy and immune therapy. The method may include treating the subject by a combination of various treatments described herein.

Multiple therapeutic strategies against bone metastasis may include the direct targeting of osteoclasts, which has been shown to be an effective method of limiting bone loss. miR-141, miR-219, miR-190, miR-33a, and miR-133a may serve as therapeutics by limiting osteoclast differentiation.

In an embodiment, a method of treating a subject suffering from a bone degenerative disease is provided. The method may include obtaining a test sample from the subject. The method may include determining an expression level of at least one osteoclast differentiation marker in the test sample. The at least one osteoclast differentiation marker may be one or more of miRNAs or sICAM1. The method may include comparing the expression level of the at least one osteoclast differentiation marker in the test sample to the expression level of the at least one osteoclast differentiation marker in a reference sample. An increase in the expression level of the at least one osteoclast differentiation marker in the test sample compared to the reference sample may indicate the subject will be a positive responder. A positive responder is a subject who positively responds to treatment with any one of the pharmaceutical composition described herein. The positive responder may experince amelioration of symptoms of bone metastasis, remission, abatement, slowing the rate of osteolytic bone degeneration or decline, or making osteolytic bone degeneration less debilitating. A finding of a decrease in the expression level of the at least one osteoclast differentiation marker in the test sample compared to the reference sample may indicate the subject will be a negative responder. A negative responder is a subject who does not respond to treatment with any one of the pharmaceutical compositions described herein, or responds to treatment but not at the level of the positive responder. The method may further include recommending or conducting at least one of: treating the subject with the any one of pharmaceutical compositions herein if the subject is determined to be a positive responder, or treating the subject with one or more of adjuvant therapy, chemotherapy, immune therapy, radiation therapy, gene therapy, surgery, use of prosthetics, vaccination, use of hormones, cytokines, vitamins, chemokines, antibiotic therapy, transplantation, radiation therapy or immunotherapy if the subject is determined to be a negative responder. If the expression level of the one or more miRNAs is elevated compared to the reference, the step of treating the subject with the any of the pharmaceutical compositions herein may include is treating the subject with the pharmaceutical composition including a therapeutic agent that includes a first nucleic acid with at least 70, 72, 75, 80, 85, 90, 91, 92, 93, 94, 95, 96, 97, 98, 99, or 100% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86). The pharmaceutical composition may include at least one additional therapeutic agent. The the at least one additional therapeutic agent may include a second nucleic acid with least 70, 72, 75, 80, 85, 90, 91, 92, 93, 94, 95, 96, 97, 98, 99, or 100% identity to a sequence is selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86). The sequence selected for the second nucleic acid may be different than the sequence selected for the first nucleic acid. The at least one additional therapeutic agent may include a bone metastasis therapeutic agent, a breast cancer therapeutic agent, or an anti-estrogen therapeutic agent.

If the expression level of sICAM1 is elevated compared to the reference sample, the step of treating the subject with the any one of the pharmaceutical compositions described herein may include treating the subject with a pharmaceutical composition that includes an antibody. The antibody may be specific to ICAM-1. The antibody may be specific to sICAM-1. The antibody may be a monoclonal antibody that binds sICAM-1. The antibody may be a monoclonal antibody that binds an sICAM1 receptor. The sICAM1 receptor may be a β2 integrin. The β2 integrin may be one of αLβ2 integrin and αMβ2 integrin.

A correspondent osteoclast differentiation marker may be an osteoclast differentiation marker of a same identity to one being analyzed in a test sample. The reference sample may be selected from the group consisting of: the expression level of the correspondent osteoclast differentiation marker in a second test sample obtained from a healthy individual, a reference value obtained in prior sets of samples from healthy individuals, and the expression level of the correspondent osteoclast differentiation marker in a second test sample that has been exposed to an agent inducing osteoclast differentiation. The agent may be selected from the group consisting of: a tumor-conditioned medium, a receptor activator of NF-κB ligand, and sICAM1. The at least one osteoclast differentiation marker may be miR-33a, miR-133a, miR-190, miR-219, miR-141, and sICAM1. The at least one osteoclast differentiation marker may be an miRNA. The miRNA may be miR-33a, miR-133a, miR-190, miR-219, or miR-141. The miRNA may be a combination of miR-33a, miR-133a, miR-190, miR-219, or miR-141. The at least one osteoclast differentiation marker may include a nucleic acid with at least 70, 72, 75, 80, 85, 90, 91, 92, 93, 94, 95, 96, 97, 98, 99, or 100% identity to a sequence selected from the group consisting of: miR-33a (SEQ ID NO; 74), miR-133a (SEQ ID NO: 61), miR-190 (SEQ ID NO: 64), miR-219 (SEQ ID NO: 66), and miR-141 (SEQ ID NO: 69). The miRNA may be miR-16 or miR-378. The miRNA may be a combination of mi-R-16 and miR-378. The one or more miRNA may include a sequence with at least 70, 72, 75, 80, 85, 90, 91, 92, 93, 94, 95, 96, 97, 98, 99, or 100% identity to a sequence of SEQ ID NO: 75 or SEQ ID NO: 76.

The test sample may include but is not limited to cells or serum.

In an embodiment, a method for diagnosing whether a subject has bone metastasis is provided. The method may include obtaining a first test sample from the subject afflicted with cancer and a second test sample from a healthy individual. The method may include determining an expression level of at least one osteoclast differentiation marker in the first test sample and the second test sample. The method may include comparing the expression level of the at least one osteoclast differentiation marker in the first test sample to the expression level of the corresponding osteoclast differentiation marker in the second test sample. The method may include diagnosing the subject as having bone metastasis if the expression level of the at least one osteoclast differentiation marker in the first test sample is elevated compared to the corresponding osteoclast differentiation marker in the second test sample. The at least one osteoclast differentiation marker may be selected from the group consisting of: miR-33a, miR-133a, miR-141, miR-190, mi-R-219, and sICAM1. The at least one osteoclast differentiation marker may be one or more of miR-33a, miR-133a, miR-141, miR-190, or mi-R-219. The at least one osteoclast differentiation marker may be a nucleic acid having at least 90% identity to a sequence selected from the group consisting of: a nucleic acid with at least 70, 72, 75, 80, 85, 90, 91, 92, 93, 94, 95, 96, 97, 98, 99, or 100% identity to a sequence selected from the group consisting of: miR-33a (SEQ ID NO; 74), miR-133a (SEQ ID NO: 61), miR-190 (SEQ ID NO: 64), miR-219 (SEQ ID NO: 66), and miR-141 (SEQ ID NO: 69). The at least one osteoclast differentiation marker may be miR-16 or miR-378. The at least one osteoclast differentiation marker may be a combination of miR-16 and miR-378. The at least one osteoclast differentiation marker may include a nucleic acid with at least 70, 72, 75, 80, 85, 90, 91, 92, 93, 94, 95, 96, 97, 98, 99, or 100% identity to a sequence of SEQ ID NO: 75 or SEQ ID NO: 76. The method may further include recommending treating the subject having bone metastasis with any pharmaceutical composition described herein.

The following list includes particular embodiments of the present invention. But the list is not limiting and does not exclude alternate embodiments, as would be appreciated by one of ordinary skill in the art.

### EMBODIMENTS

1. A pharmaceutical composition comprising a therapeutic agent including a first nucleic acid with at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86), and at least one additional therapeutic agent.

2. The pharmaceutical composition of embodiment 1, wherein the first nucleic acid has a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86).

3. The pharmaceutical composition of embodiment 1, wherein the at least one additional therapeutic agent includes a second nucleic acid with at least 90% identity to a sequence is selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86), wherein the sequence selected for the second nucleic acid is different than the sequence selected for the first nucleic acid.

4. The pharmaceutical composition of embodiment 3, wherein the second nucleic acid has a sequences selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86).

5. The pharmaceutical composition of any one or more of the preceding embodiments, wherein the at least one additional therapeutic agent is selected from the group consisting of: a bone metastasis therapeutic agent, a breast cancer therapeutic agent, and an anti-estrogen therapeutic agent.

6. The pharmaceutical composition of any one or more of the preceding embodiments, wherein the at one additional therapeutic agent is selected from the group consisting of: bisphosphonates, alendronate, ibandronate, risedronate, pamidronate, a RANKL antibody, a sclerostin antibody, methotrexate, paclitaxel, or paclitaxel albumin-stabilized nanoparticle formulation, doxorubicin or doxorubicin hydrochloride, fluorouracil or 5-fluorouracil, everolimus, anastrozole, docetaxel, epirubicin hydrochloride, toremifene, fulvestrant, letrozole, gemcitabine hydrochloride, ixabepilone, megestrol acetate, cyclophosphamide, toremifene, lapatinib or lapatinib ditosylate, capecitabine, zoledronic acid, goserelin acetate, exemestane, tamoxifen, trastuzumab, ado-trastuzumab emtansine, and pertuzumab.

7. The pharmaceutical composition of any one or more of the preceding embodiments, wherein the at least one additional therapeutic agent includes an antibody that specifically binds sICAM1.

8. The pharmaceutical composition of any one or more of the preceding embodiments, wherein the at least one additional therapeutic agent includes an antibody that specifically binds an sICAM1 receptor.

9 The pharmaceutical composition of embodiment 8, wherein the sICAM1 receptor includes a 132 integrin.

10. The pharmaceutical composition of embodiment 9, wherein the β2 integrin is one of αLβ2 integrin and αMβ2 integrin

11. The pharmaceutical composition of any one of embodiments 7-10, wherein the antibody is a monoclonal antibody.

12. The pharmaceutical composition of any one or more of the preceding embodiments further comprising a pharmaceutically acceptable carrier.

13. The pharmaceutical composition of any one or more of the preceding embodiments further comprising a nanocarrier.

14. The pharmaceutical composition of embodiment 13, wherein the nanocarrier is selected from the group consisting of: lipid nanoparticles, liposomes, polymer particles, ligands and polydexstrins.

15. A method of treating a subject suffering from a bone degeneration disease comprising administering a pharmaceutical composition of any one of claims 1-10 to the subject.

16. The method of embodiment 15, wherein the bone degeneration disease is selected from the group consisting of: bone metastasis, osteoporosis, and Paget disease.

17. The method of embodiment 16, wherein the bone degeneration disease is bone metastasis.

18. The method of any one or more of embodiments 15-17 further comprising assessing inhibition of bone metastasis growth in the subject before and after treatment.

19. The method of embodiment 18, wherein the assessing includes measuring a rate of bone metastasis.

20. The method of embodiment 18, wherein the assessing includes determining expression of at least one osteoclast differentiation marker gene in the subject after treatment.

21. The method of embodiment 20, wherein the at least one osteoclast differentiation marker gene is selected from the group consisting of: Mitf, Traf6, Mmp14, Calcr, Cpr, Mmp9, Itgav, Oscar, Nfatc1, Ctsk, Calcr site1, Calcr site2, Mitf site1, and Mitf site 2.

22. The method of any one of embodiments 18-21 further comprising terminating treatment if the bone metastasis growth in the subject is inhibited.

23. The method of any one or more of the embodiments 15-22, wherein the pharmaceutical composition is associated with a nanocarrier.

24. The method of embodiment 23, wherein the nanocarrier is selected from the group consisting of: lipid nanoparticles, liposomes, polymer particles, ligands and polydexstrins.

25. The method of embodiment 15, wherein the step of administering includes administering by a route selected from the group consisting of: intravenous, intraperitoneal, intramuscular, and subcutaneous injection.

26. The method of any one or more of embodiments 15-25 further comprising treating the subject with radiation therapy.

27. The method of any one or more of embodiments 15-26 further comprising treating the subject with immune therapy.

28. The method of any one or more of embodiments 15-27, wherein the subject is a mammal.

29. The method of embodiment 28, wherein the mammal is a rodent.

30. The method of embodiment 28, wherein the subject is a human.

31. A method of treating a subject suffering from a bone degenerative disease comprising:

obtaining a test sample from the subject,

determining an expression level of at least one osteoclast differentiation marker in the test sample, wherein the at least one osteoclast differentiation marker is one or more of miRNAs or sICAM1, and

comparing the expression level of the at least one osteoclast differentiation marker in the test sample to the expression level of the at least one osteoclast differentiation marker in a reference sample, wherein an increase in the expression level of the at least one osteoclast differentiation marker in the test sample compared to the reference sample indicates the subject will be a positive responder, and a finding of a decrease in the expression level of the at least one osteoclast differentiation marker in the test sample compared to the reference sample indicates the subject will be a negative responder;

the method further comprising one or more of recommending or conducting at least one of:

treating the subject with the pharmaceutical composition of any one of embodiments 1-10 if the subject the subject is determined to be a positive responder, and treating the subject with one or more of radiation therapy or immunotherapy if the subject is determined to be a negative responder.

32. The method of embodiment 31, wherein if the expression level of the one or more miRNAs is elevated compared to the reference, the step of treating the subject with the pharmaceutical composition of any one of embodiments 1-10 is treating the subject with the pharmaceutical composition of any one of embodiments 1-6.

33. The method of embodiment 31, wherein if the expression level of sICAM1 is elevated compared to the reference, the step of treating the subject with the pharmaceutical composition of any one of embodiments 1-10 includes treating the subject with the pharmaceutical composition of any one of embodiments 7-10.

34. The method of embodiment 31, wherein a correspondent osteoclast differentiation marker is an osteoclast differentiation marker of a same identity of one being compared in a test sample, and the reference sample is selected from the group consisting of: the expression level of the correspondent osteoclast differentiation marker in a second test sample obtained from a healthy individual, a reference value, and the expression level of the correspondent osteoclast differentiation marker in a second test sample that has been exposed to an agent inducing osteoclast differentiation.

35. The method of embodiment 34, wherein the agent is selected from the group consisting of: a tumor-conditioned medium, a receptor activator of NF-κB ligand, and sICAM1.

36. The method of embodiment 35, wherein the at least one osteoclast differentiation marker is selected from the group consisting of: miR-33a, miR-133a, miR-141, miR-190, mi-R-219, and sICAM1.

37. The method of embodiment 36, wherein the at least one osteoclast differentiation marker is selected from the group consisting of: miR-33a, miR-133a, miR-141, miR-190, and miR-219.

38. The method of embodiment 37, wherein the at least one osteoclast differentiation marker includes a nucleic acid having at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), and miR-141 (SEQ ID NO: 69).

39. The method of embodiment 31, wherein the correspondent osteoclast differentiation marker is an miRNA, and the miRNA is miR-16, miR-378, or a combination of mi-R-16 and miR-378.

40. The method of embodiment 39, wherein the one or more miRNA includes a nucleic acid having at least 90% identity to a sequence of SEQ ID NO: 75) or SEQ ID NO: 76.

41. The method of any one or more of embodiments 31-40, wherein the subject is a mammal, and the mammal is selected from the group consisting of a rodent, a human, a primate, and a high value agricultural animal.

42. The method of embodiment 31, wherein the test sample comprises cells and serum.

43. A method for diagnosing whether a subject has bone metastasis comprising:

obtaining a test sample from the subject afflicted with cancer;

determining an expression level of at least one osteoclast differentiation marker in the test sample;

comparing the expression level of the at least one osteoclast differentiation marker in the test sample to the expression level of the corresponding osteoclast differentiation marker in a reference sample; and

diagnosing the subject as having bone metastasis if the expression level of the at least one osteoclast differentiation marker in the test sample is elevated compared to the corresponding osteoclast differentiation marker in a reference sample.

44. The method of embodiment 43, wherein the at least one osteoclast differentiation marker is selected from the group consisting of: miR-33a, miR-133a, miR-141, miR-190, mi-R-219, and sICAM1.

45. The method of embodiment 43 or 45, wherein the at least one osteoclast differentiation marker is selected from the group consisting of: miR-33a, miR-133a, miR-141, miR-190, and miR-219.

46. The method of embodiment 45, wherein the at least one osteoclast differentiation marker includes a nucleic acid having at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), and miR-141 (SEQ ID NO: 69).

47. The method of embodiment 43, wherein the at least one osteoclast differentiation marker is an miRNA, and the miRNA is miR-16, miR-378, or a combination of mi-R-16 and miR-378.

48. The method of embodiment 47, wherein the one or more miRNA includes a nucleic acid having at least 90% identity to a sequence of SEQ ID NO: (SEQ ID NO: 75) or SEQ ID NO: (SEQ ID NO: 76).

49. The method of embodiment 43, wherein a correspondent osteoclast differentiation marker is an osteoclast differentiation marker of a same identity of one being compared in a test sample, and the reference sample is selected from the group consisting of: the expression level of the correspondent osteoclast differentiation marker in a second test sample obtained from a healthy individual, a reference value, and the expression level of the correspondent osteoclast differentiation marker in a second test sample that has been exposed to an agent inducing osteoclast differentiation.

50. The method of any one or more of embodiments 43-49 further comprising recommending treating the subject having bone metastasis with a pharmaceutical composition of any one of embodiments 1-10.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### Materials and Methods

Cell Lines and Cell Culture

HeLa and RAW264.7 cells (American Type Culture Collection, ATCC) were maintained in Dulbecco's modified Eagle's medium (DMEM) supplemented with 10% fetal bovine serum (FBS), penicillin/streptomycin (GIBCO), and fungizone. MOCP-5 cells were maintained in αMEM with 10% FBS and antibiotics. MDA-MB-231 parental cells (ATCC) and all sublines (SCP2, SCP4, SCP6, SCP28, SCP46) were maintained in DMEM with 10% FBS and antibiotics. The murine 4T1 and 4T1.2 cells, and human TSU-Pr1, and TSU-PR1-B2 cells were similarly maintained in DMEM with 10% FBS and antibiotics. For osteoclast differentiation assays, cells were treated with indicated concentration of RANKL in DMEM plus 10% FBS, with media changes performed every 2 days. Primary osteoclasts were isolated from bone marrow cells flushed from the tibia of 6-week-old wild type Balb/c mice and filtered through a 70 μM cell-strainer before overnight culture in aMEM with 10% FBS. The following day 1×10⁶ non-adherent cells were plated in 6-well plates supplemented with 50 ng/mL M-CSF for 2 days, followed by 50 ng/mL RANKL for an additional 4-5 days, with media changes every 2 days. Human osteoclasts were obtained from Lonza (2T-110) and differentiated by treatment with 33 ng/ml M-CSF and 66 ng/ml RANKL. CM was collected from the indicated sub-confluent tumor cells grown in DMEM with 10% DMEM for 24 hours. The CM was passed through a 0.2 μm filter before addition to 1×10⁵ RAW264.7 or MOCP-5 cells in a 6-well plate. The media was replaced with fresh CM daily. Cells were TRAP stained on day 6 using a leukocyte acid phosphatase kit (Sigma) and TRAP⁺-multineucliated cells were quantified as mature osteoclasts.

RNA Isolation and miRNA Microarray

RNA from cell lines was isolated using a miRVana miRNA isolation kit (AMBION®). Total RNA from mouse bleeds was isolated using Trizol LS (Invitrogen) according to manufacturers instructions. For microarrays, 5 μg total RNA was labeled using the NCode Rapid Labeling System (Invitrogen) and hybridized to custom arrays. The arrays were analyzed using the G2565BA scanner (Agilent Technologies), and median fluorescent intensities were obtained after subtracting background. To identify differential miRNA expression between samples, the median fluorescent intensities were normalized using the median expression values within the array and log 2 values analyzed.

Quantitative Real-Time PCR

mRNA was analyzed by synthesizing cDNA using the Superscript III First-strand kit (Invitrogen) and qPCR performed using the Power SYBR® green PCR master mix (Applied Biosystems). Mature miRNAs were reverse transcribed using the TaqMan Reverse Transcription Kit (Applied Biosystems) followed by real-time PCR using TaqMan miRNA assays (Applied Biosystems). All analysis was performed using an ABI 7900HT PCR machine according to the manufacturer's instructions. A standard curve was created from serial dilutions from cDNA for each gene of interest. Values were normalized by the expression of GAPDH or RNU6B in each sample. The primers used are listed in the table below:

| Gene | Species | Forward | Reverse |
|---|---|---|---|
| Calcr | Mouse | GCAACGCTTTCACTTCTGAGA | GTTCCCACTGCATTGTCCACA |
| | | (SEQ ID NO: 1) | (SEQ ID NO: 2) |
| Tnfrsf11a | Mouse | CACTCCCACCCTGAGATTTGT | CATCGTCTGCACGGTTCTG (SEQ |
| | | (SEQ ID NO: 3) | ID NO: 4) |
| Nfatc1 | Mouse | GACCCGGAGTTCGACTTCG | TGACACTAGGGGACACATAACTG |
| | | (SEQ ID NO: 5) | (SEQ ID NO: 6) |
| Csf1 | Mouse | TGTCATCGAGCCTAGTGGC | CGGGAGATTCAGGGTCCAAG |
| | | (SEQ ID NO: 7) | (SEQ ID NO: 8) |
| Itgav | Mouse | CCGTGGACTTCTTCGAGCC | CTGTTGAATCAAACTCAATGGGC |
| | | (SEQ ID NO: 9) | (SEQ ID NO: 10) |
| Fosl2 | Mouse | CCAGCAGAAGTTCCGGGTAG | GTAGGGATGTGAGCGTGGATA |
| | | (SEQ ID NO: 11) | (SEQ ID NO: 12) |
| Sfpi1 | Mouse | ATGTTACAGGCGTGCAAAATGG | TGATCGCTATGGCTTTCTCCA |
| | | (SEQ ID NO: 13) | SEQ ID NO: 14) |
| Mitf | Mouse | ACTTTCCCTTATCCCATCCACC | TGAGATCCAGAGTTGTCGTACA |
| | | (SEQ ID NO: 15) | (SEQ ID NO: 16) |
| Ctsk | Mouse | GAAGAAGACTCACCAGAAGCAG | TCCAGGTTATGGGCAGAGATT |
| | | (SEQ ID NO: 17) | (SEQ ID NO: 18) |
| Gapdh | Mouse | AGGTCGGTGTGAACGGATTTG | TGTAGACCATGTAGTTGAGGTCA |
| | | (SEQ ID NO: 19) | (SEQ ID NO: 20) |

Luciferase Reporter Assay

For NFκB reporter assays the pGL4.32 [luc2P/NF-κB-RE/Hygro] vector (Promega) was co-transfected with a Renilla-luciferase plasmid into cells. After 24 hours, cells were treated with 50 ng/ml RANKL, 50 ng/ml sICAM1, or 10 ng/ml TNFα for analysis at indicated timepoints. Wildtype and mutant 3′UTRs were PCR amplified from mouse genomic DNA. The 3′UTRs were cloned into the pMIR-REPORT vector (AMBION®) downstream of firefly luciferase. Mutations in miRNA target sites were generated using the QuikChange Multi site-directed mutagenesis kit (Stratagene). 5×10⁴ HeLa cells were plated in 24-well plates 24 hours prior to transfection. 200 ng of reporter plasmid was co-transfected with the renilla-luciferase control plasmid and 10 pM pre-miRNA precursor or precursor control (AMBION®) using Lipofectamine 2000 (Invitrogen). Cells were lysed 24 hours after transfection and assayed for luciferase activity using the Glomax 96 Luminometer (Promega). Primer sequences for cloning or mutagenesis are listed below:

| 3'UTR | | |
|---|---|---|
| reporter | Forward | Reverse |
| Calcr | AACACAGCATCGTGATCACTGAG | CATAATTTGGGCAGAACTATGTGC (SEQ |
| | (SEQ ID NO: 21) | ID NO: 22) |
| Acp 5 | ACACCACGAGAGTCCTGCTTGT (SEQ | TTCTGGAGACAAAGCATTGTTTTATTG |
| | ID NO: 23) | (SEQ ID NO: 24) |
| Mmp9 | AGGGCTCCTTCTTTGCTTCAAC (SEQ | GACTGCCAGGAAGACACTTGGT (SEQ |
| | ID NO: 25) | ID NO: 26) |
| Mitf | CGAGCCTGCCTTGCTCTG (SEQ ID | GCATCATGCTCTGGGTATGACC (SEQ |
| | NO: 27) | ID NO: 28) |
| Itgav | TTCAACAGTTCCTCAAGGCCT (SEQ | CATGTTGTAAATCAAGCAGAACACTG |
| | ID NO: 29) | (SEQ ID NO: 30) |
| Ctsk | CAGCCAAATCCATCCTGCTCT (SEQ | AAAGTGCAAAGAAGGGAAGACAA (SEQ |
| | ID NO: 31) | ID NO: 32) |
| Mmp14 | TACCACAAGGACTTTGCCTCTG (SEQ | ATCTAGCCGAACTGCCAGCAC (SEQ ID |
| | ID NO: 33) | NO: 34) |
| Nfatc1 | ACATTGGAGCACTCAGTTCAGC (SEQ | CGTTTCCTTTTCAATGACAGTCC (SEQ |
| | ID NO: 35) | ID NO: 36) |
| Cpe | GTAGTAAGATGCAATGTGGCTC | CTTTATTCATCAGAATGCAATTC (SEQ |
| | (SEQ ID NO: 37) | ID NO: 38) |
| Fosb | CAAACCCGCAAGGAACA AG (SEQ ID | GGCGACAGTGCAGAACCAAG (SEQ ID |
| | NO: 39) | NO: 40) |
| Traf6 | TCAGTGAGGTCACGAGCCACTTC | AGCTGAGTGTCCACACAACCGT (SEQ |
| | (SEQ ID NO: 41) | ID NO: 42) |
| Oscar | CAACCTGAGTGGCGGAGAA (SEQ ID | GCAGCAGCAAGATTTATTGTGTAGA |
| | NO: 43) | (43SEQ ID NO: 44) |
| Calcr-141mut | AGGTATTTTTCCAATCAgAcTcTaATG | GTAAACAAAAATAGAACAATACTACATT |
| | TAGTATTGTTCTATTTTTGTTTAC | AGAGTCTGATTGGAAAAATACCT (SEQ |
| | (SEQ ID NO: 45) | ID NO: 46) |
| Mift-141mut1 | ATCATCATTTTTTTCtGacTaTGTATT | ATATTCTTACAAATTAATACATAGTCAG |
| | AATTTGTAAGAATAT (SEQ ID NO: | AAAAAAATGATGAT (SEQ ID NO: 48) |
| | 47) | |
| Mitf-141mut2 | GGGACTGGTTAAGCCAAGACGCtGac | AAACAGTGAGCAAATAGTCAGCGTCTT |
| | TaTTTGCTCACTGTTT (SEQ ID NO: | GGCAAGGCTTAACCAGTCCC (SEQ ID |
| | 49) | NO: 50) |
| Mitf-219mut | ACCATAGTGTTCACCGATtCtAaCtTA | GAAGAGAAAAACAGACAATGCTAAGTT |
| | GCATTGTCTGTTTTTCTCTTC (SEQ | AGAATCGGTGAACACTATGGA (SEQ ID |
| | ID NO: 51) | NO: 52) |
| Traf6-219mut | TCTTCTGTTGCTTGCAAACACtAtAaC | GAGCTAAATCACTCGTTTGGGCTGGTG |
| | ACCAGCCCAAACGAGTGATTTAGCT | TTATAGTGTTTGCAAGCAACAGAAGA |
| | C (SEQ ID NO: 53) | (SEQ ID NO: 54) |
| Mitf-133amut | CCTCCGTTTAGAGTcCaCtAtATACCA | GGTGGATAATTTGGTATATAGTGGACT |
| | AAATTATCCACC (SEQ ID NO: 55) | CTAAACGGAGG (SEQ ID NO: 56) |
| Mmp14- | AGGGGGCAGGAGGcGtCgAtAAAGGA | GTCCTCATTTTCCTTTATCGACGCCTCC |
| 133amut | AAATGAGGAC (SEQ ID NO: 57) | TGCCCCCT (SEQ ID NO: 58) |
| Calcr-190mut | CCTCCCAGGAACCGAtCtTtTgATTTG | TTCACTGAATAATTCTTCACAAATCAAA |
| | TGAAGAATTATTCAGTGAA (SEQ ID | AGATCGGTTCCTGGGAGG (SEQ ID NO: |
| | NO: 59) | 60) |

Bone Resorption Assay

5×10⁴ pre-osteoclast cells were seeded onto 1 mm thick slices of bovine bone cut using a diamond saw and sterilized by immersion in 70% ethanol. Cells were cultured for 10 days as indicated in 6-well plates, transferred to fresh plates and washed to remove osteoclasts. Bone slices were stained in a solution of 1% toludine blue in 0.5% sodium tetraborate, destained in PBS plus 1% Triton X-100, and imaged by light microscopy.

Histomorphometric Analysis and Immunohistochemical Staining

Hindlimb bones were excised from mice at the end point of each experiment, immediately following X-ray and BLI. Hind limb bones were fixed in 10% neutral-buffered formalin, washed and decalcified in a solution of 10% EDTA for 2 weeks. One limb from each mouse was stored in 70% ethanol for μCT analysis and Von Kossa staining, while the other limb was embedded in paraffin for hematoxylin and eosin (H&E), tartrate-resistant acid phosphatase (TRAP), or immunohistochemical staining. Non-decalcified sections were embedded in resin using the Osteo-bed Bone Embedding Kit (Sigma Aldrich, EM0200) before sectioning, followed by Von Kossa staining. Briefly, sections were rehydrated to H₂O, followed by submersion in 1% silver nitrate and exposure to UV light for 5 minutes. Sections were then incubated in 2.5% Sodium Thiosulphate, followed by counterstaining with Nuclear Fast Red and dehydration through Xylene. Histomorphometric analysis was performed on H&E stained bone metastasis sections using the Zeiss Axiovert 200 microscope and the AxioVision software version 4.6.3 SP1. For quantitative analysis of lesion area, a 5× objective was used to focus on the tumor region of interest and images were acquired using an AxioCamICc3 camera set to an exposure of 50 ms. Lesions that were larger than the field of view were quantified by acquiring multiple images to encompass the entire lesion and combined in Adobe Photoshop CS5. Lesion area was quantified by outlining the region(s) of interest and quantifying lesion area. Osteoclast number was assessed as multinucleated TRAP⁺ cells along the tumor-bone interface and reported as number/mm of interface as previously reported (Sethi et al., 2011). Immunohistochemical analysis was performed with heat-induced antigen retrieval and an osteocalcin antibody (ABcam, AB93876). A biotinylated secondary antibody was used with Vectastain ABC Kit (Vector Laboratories) and DAB detection kit (Zymed) to reveal the positively stained cells; nuclei were counterstained with hematoxylin

X-Ray Analysis and Quantification

Bone remodeling in mice was assessed by X-ray radiography.

Anesthetized mice were placed on singlewrapped films (X-OMAT AR, Eastman Kodak) and exposed to X-ray radiography at 35 kV for 15 s using a MX-20 Faxitron instrument. Films were developed using a Konica SRX-101A processor. Changes in bone remodeling and osteolytic lesions (radiolucent lesions) in the hindlimbs of mice were identified and quantified using the ImageJ software (National Institutes of Health)

Western Blot Analyses

SDS lysis buffer (0.05 mM Tris-HCl, 50 mM BME, 2% SDS, 0.1% Bromophenol blue, 10% glycerol) was used to collect protein from cells. Samples were sonicated and heat denatured protein was equally loaded, separated on a 10% SDS-page gel, transferred onto a pure nitrocellulose membrane (BioRad), and blocked with 5% milk. Primary antibodies for immunoblotting included: anti-IκBα (1:1000 dilution, Cell signaling, 44D4), anti-p-p65 (1:500 dilution, Santa Cruz Biotechnology), anti-Pu.1 (5 μg/ml, Abcam, AB88082), anti-Nfatc1 (1 μg/ml, Abcam, AB25916), anti-Ctsk (4 μg/ml, Abcam, AB19027), anti-β-actin (1:5000 dilution, ABcam), for loading control. Membranes were incubated with horseradish peroxidase (HRP)-conjugated anti-mouse secondary antibody (1:2000 dilution, GE Healthcare) or anti-rabbit secondary antibody (1:2000 dilution, GE Healthcare) for 1 h and chemiluminescence signals were detected by ECL substrate (GE Healthcare).

Antibodies and Recombinant Proteins

Recombinant ICAM1 (R&D Systems, 796-IC-050) was dissolved in PBS. Cells were seeded on a 6-well plate and treated with sICAM1, CM, or RANKL. Anti-murine β2 antibody (CD18, BD Biosciences, 555280) or ICAM1 antibody (ABcam, ab25375) was administered at a concentration of 50 μg/mL as described. Recombinant rat Jagged1/Fc chimera (R&D systems) was dissolved in PBS and plated at a concentration of 0.5 mg/ml in 6-well plates that had been pre-coated with anti-Fc antibody for 1 hour and blocked with DMEM containing 10% FBS for 2 hours.

Flow Cytometry

Cultured cells were resuspended in FACS buffer (PBS supplemented with 5% newborn calf serum) and filtered through 70 mm nylon cell strainers before flow cytometric analysis on a FACSort instrument (BD Biosciences). The following antibodies were selected to label cells for 30 minutes on ice: mouse anti-CD11a (BD, 610826), rat anti-CD11b (Abcam, ab8878), rat anti-CD18 (BD, 555280), donkey anti-rat-alexa594 (Invitrogen, 982443), goat anti-mouse-alexa488 (Invitrogen, A11029).

Chemotaxis Assay of RAW264.7 Cells

Chemotaxis assay was performed as described (Lu et al., 2011) with the following modifications: 10⁵ RAW264.7 cells in 100 μl of 0.5% BSA in DMEM were seeded into the upper chamber of 8 μm pore transwell inserts (BD Bioscience) in a 24-well plate. 300 μl of 0.5% BSA in DMEM containing a series of concentrations of recombinant ICAM1 (R&D Systems) was added to the bottom chamber. After 8 h in culture, methanol was added to the bottom chamber to fix migrated cells. Cells in the upper chamber were removed with a cotton swab and stained for 30 mins in 0.2% crystal violet for quantification using a microscope.

sICAM-1/CD54 and NTX Determinations from Patient Serum

sICAM-1 levels were determined using a quantitative sandwich enzyme immunoassay technique (Quantikine; R&D System, Minneapolis, Minn., USA) according to the manufacturer's instructions. All samples were tested in duplicate. The optical density of serum samples was compared with standard curve of sICAM-1 concentrations and was quantified. NTX levels were measured by a competitive-inhibition enzyme-linked immunosorbent assay (ELISA/EIA) (Osteomark, Princeton, N.J.). The assays were performed following the manufacturers' instructions. All samples were tested in duplicate for both markers, with all samples from the same individuals analyzed on the same experimental plate.

Murine and Human RANKL ELISA

Quantitative levels of murine or human RANKL in the conditioned media of cultured cells were determined in triplicate by ELISA according to the manufacturer's protocol (Mouse RANKL Quantikine ELISA kit and Human RANKL DuoSet, R&D systems).

Micro CT Analysis

Femurs and tibias were scanned using the INVEON PET/CT (Siemens Healthcare) at the Preclinical Imaging Shared Resource of Cancer Institute of New Jersey. The X-Ray tube settings were 80 kV and 500 μA and images were acquired at the highest resolution without CCD binning, resulting in a voxel size of 9.44 μm. A 0.66° rotation step through a 195° angular range with 6500 msec exposure was used. The images were reconstructed with Beam Hardening Correction and Hounsfield calibration before being analyzed using the INVEON Research Workplace software (Siemens Healthcare). After processing with a 3D Gaussian filter to reduce noise ROI's were manually segmented that corresponded to the cortical and trabecular bone regions. Cortical regions of interest comprised of 470 μm thick regions (50 slices, 9.4 μm thickness for each slice) located 3 mm distal to the growth plate, while 300 μm thick trabecular sections began 0.27 mm distal to the growth plate.

Tumor Xenografts and Bioluminescence Analysis

All procedures involving mice and experimental protocols were approved by Institutional Animal Care and Use Committee (IACUC) of Princeton University. For bone metastasis studies, 1×10⁵ tumor cells were injected into the left cardiac ventricle of anesthetized female athymic Ncr-nu/nu. MiRNA precursors (10 μg/mouse in 100 μl PBS, Applied Biosystems) and Zometa (100 μg/kg) were injected intravenously. Development of metastases was monitored by measuring photon flux of BLI signals in the hindlimbs of mice after retro-orbital injection of 75 mg/kg D-Luciferin and image acquisition using the Xenogen IVIS 200 Imaging System. Data were normalized to the signal on day 0. X-ray examination was performed as previously described (Kang et al., 2003).

Analysis of Primary Tumors and Bone Metastases

Women with resected breast cancer were selected from patients followed from 1995 to 2010 in IRCCS IRST, Meldola, Italy. Tumor specimens were de-identified and were considered exempt samples in accordance with the institutional review board of the Local Ethic Committee, Forli, Italy. Tumor specimens were fixed in formalin and embedded in paraffin. Tissues collected were 13 matched primary breast tumors and 13 bone metastatic tissues. Total RNA was collected from 20 μm thick sections from formalin-fixed paraffin embedded (FFPE) tissue blocks using the FFPE RNA/DNA Purification kit (Norgen) according to the manufacturer's instructions.

Serum Case Series and Sample Collection

Breast cancer patients with no evidence of disease for at least 5 years (16 patients) and patients at first diagnosis of bone metastases (38 patients, radiologically confirmed) were recruited by the Osteoncology and Rare Tumors Center of IRCCS IRST (Meldola, Italy) from January 2007 to December 2009. A group of 41 healthy donors was also enrolled in the study. Informed consent was obtained from all subjects in accordance with the protocol approved by the institutional review board of the Local Ethic Committee, Forli, Italy. A 5 ml venous blood sample from donors and patients was collected in tubes without anticoagulant and centrifuged at 2,500 rpm for 15 minutes at room temperature then stored at −80° C. until processing. Total RNA from 400 μl of serum was extracted using the miRVana miRNA isolation kit (AMBION®) according to the manufacturer's instructions.

Statistical Analysis

Results are presented as average ±standard deviation or as average ±standard error of the mean (SEM), as indicated in figure legends. BLI signals were analyzed by nonparametric Mann-Whitney test. For serum markers analysis, in the absence of internationally available cut off values for markers, the cut off values maximally discriminating between patients with no evidence of disease and BM patients were identified using receiver operating characteristic (ROC) curve analysis. Sensitivity and specificity were calculated and their statistical significance were analyzed by chi-square test comparing BM patients versus patients with no evidence of disease or versus healthy donors. The correlation between serum levels of the markers was assessed with the Spearman rank test. The diagnostic relevance of the combinations NTX-mir16 and NTX-sICAM1 considered as continuous variables (subjected to natural logarithmic transformation) was analyzed by the logistic regression model. The linear predictor or logit resulting from the model was used as a new diagnostic test on which the ROC curve was calculated (Flamini et al., 2006). Statistical analysis was done using SPSS software. All other comparisons were analyzed by unpaired, two-sided, independent Student's t test without equal variance assumption, unless otherwise described in figure legends.

Accession number. The raw and normalized microarray data have been deposited in the Gene Expression Ominbus (GEO) database under accession number GSE44936.

### Example 2

### Conditioned Media from Bone-Metastatic Cancer Cells Induces Osteoclast Differentiation

It was previously shown that the murine pre-osteoclast cell lines RAW264.7 and MOCP-5 can be induced to differentiate into mature, multi-nucleated osteoclasts through the addition of 20-50 ng/ml RANKL (Sethi et al., 2011).

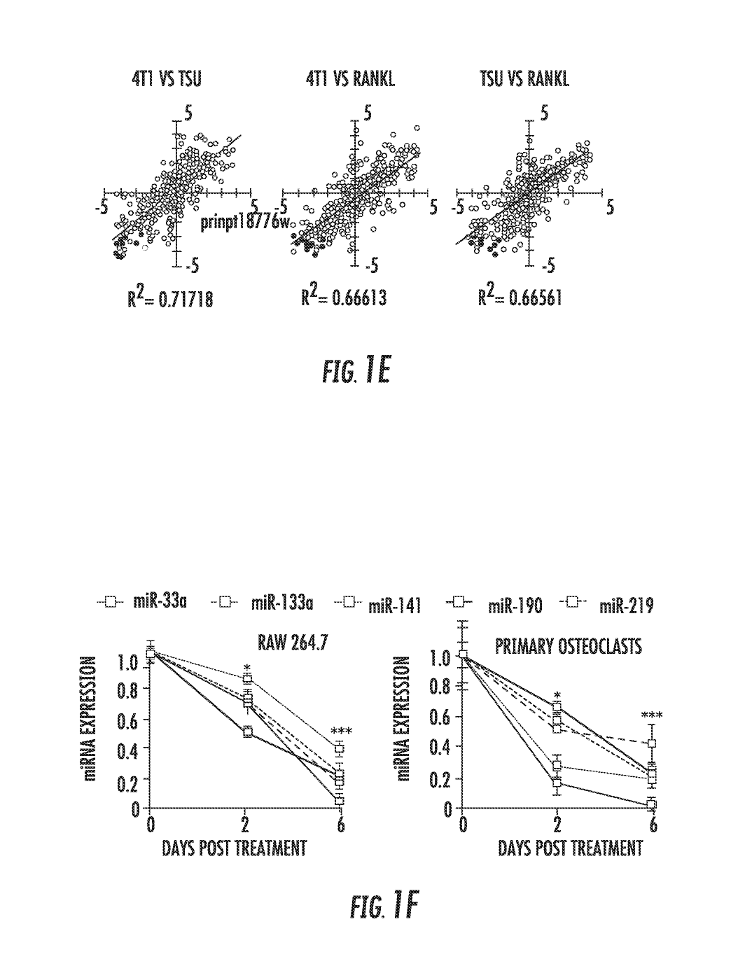

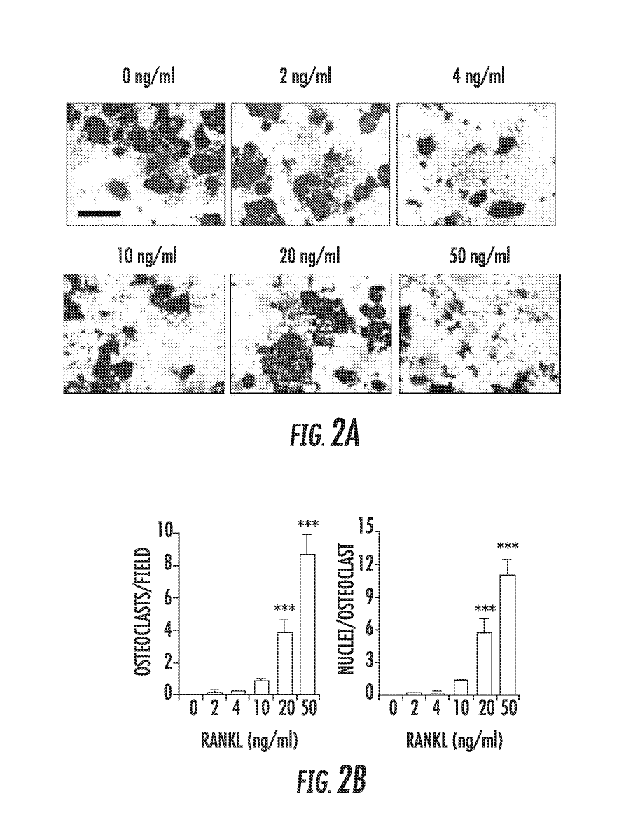

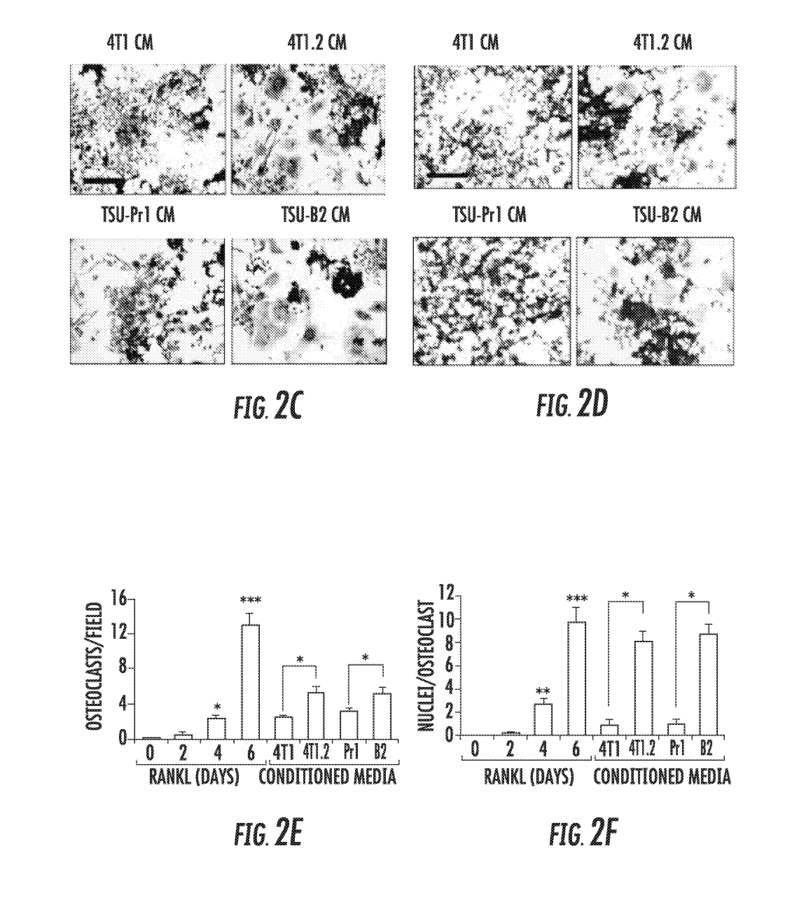

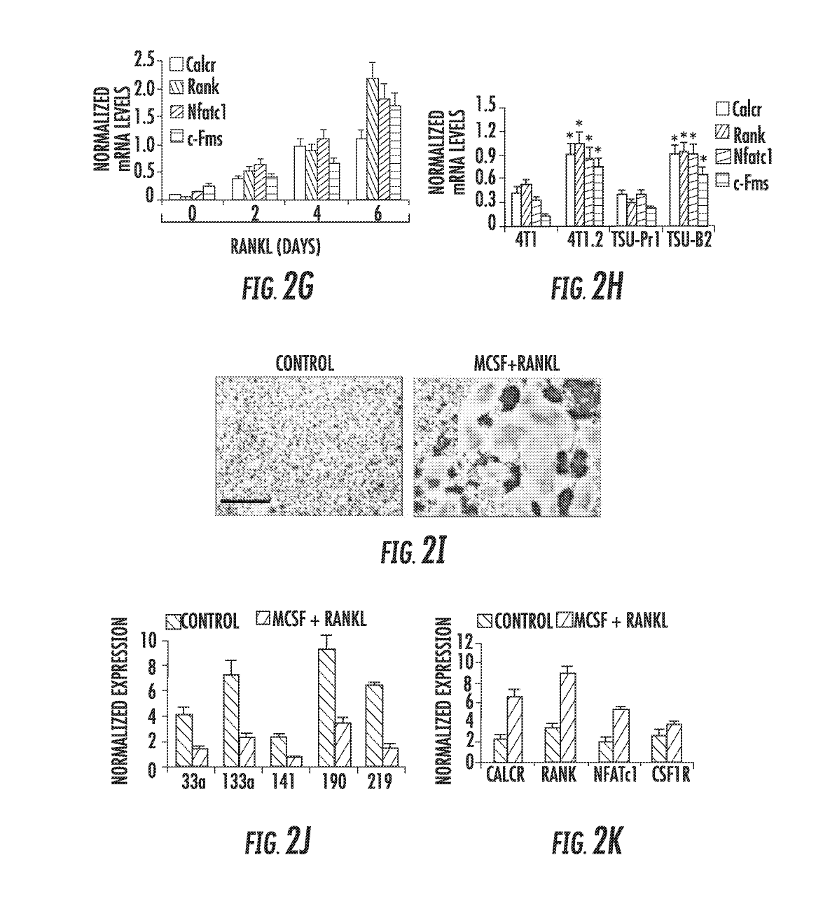

FIG. 2A illustrates representative images of TRAP stained RAW264.7 cells treated with 0 ng/ml, 2 ng/ml, 4 ng/ml, 10 ng/ml, 20 ng/ml and 50 ng/ml RANKL for 6 days. Scale bar represents 400 μm. FIG. 2B illustrates quantification of TRAP⁺ osteoclasts and nuclei per osteoclast from experiments shown in FIG. 2A. Referring to FIG. 2A, it was observed that treatment with 50 ng/ml RANKL resulted in the highest number of mature osteoclasts. These results were statistically significant with p<0.001.

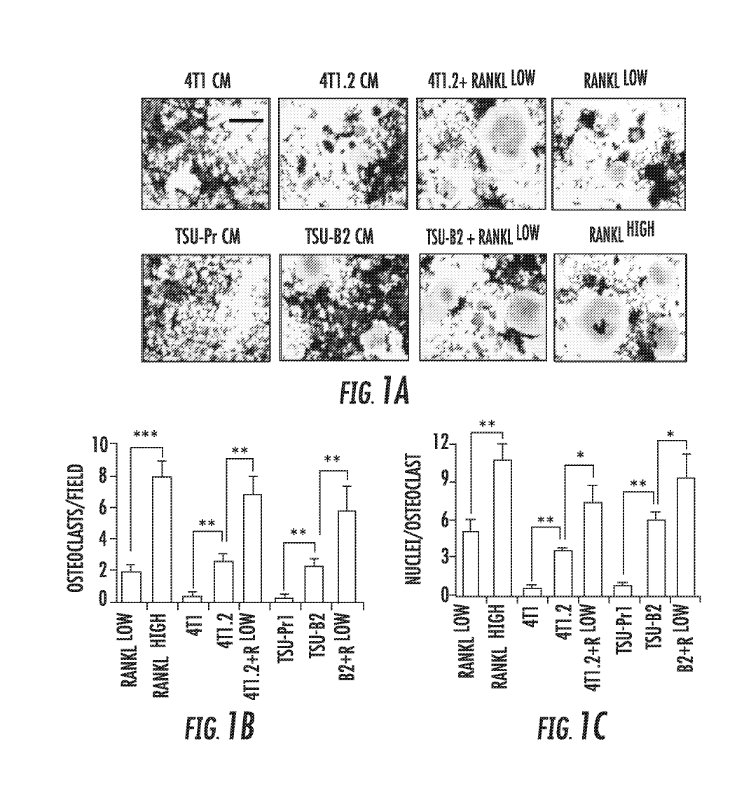

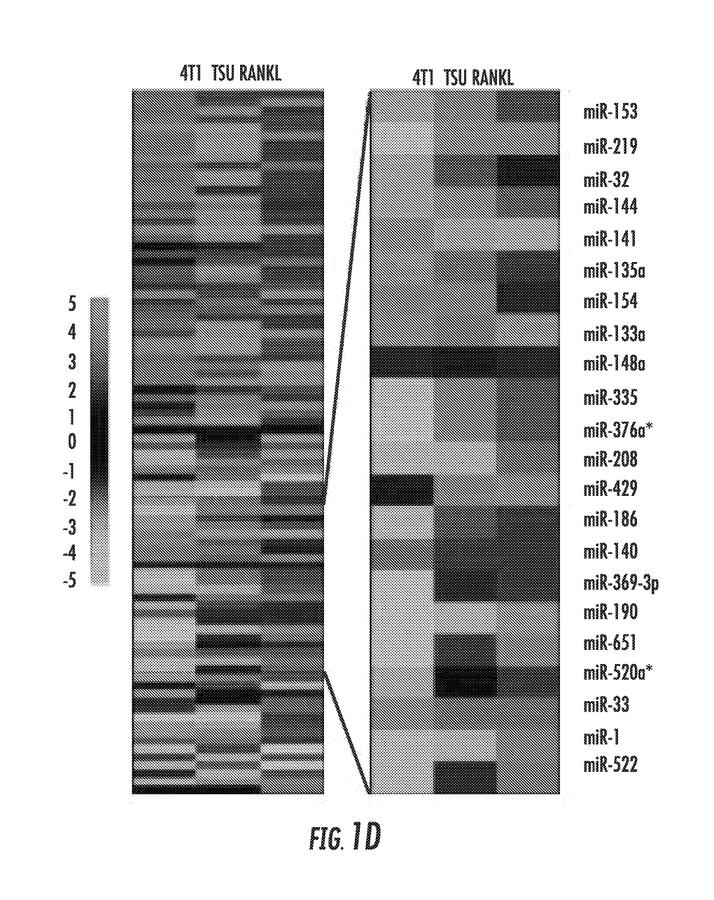

To examine the potential for tumor conditioned media (CM) to induce osteoclast differentiation, RAW264.7 or MOCP-5 pre-osteoclast cells were treated with CM from two pairs of cancer cell lines with differing bone metastasis capabilities: (1) the highly metastatic 4T1.2 mouse mammary tumor cell line and weakly metastatic 4T1 parental line (Lelekakis et al., 1999); and (2) the highly metastatic TSU-Pr1-B2 human bladder cancer cell line and the weakly metastatic TSU-Pr1 parental line (Chaffer et al., 2005). FIGS. 1A-1F illustrate that RANKL and conditioned media of highly bone metastatic cancer cell lines induce similar changes in miRNA expression in osteoclasts. FIG. 1A illustrates representative images of RAW264.7 cells stained for TRAP after treatment for 6 days with conditioned media from indicated cell lines, or conditioned media with 20 ng/ml RANKL (RANKL^{Low}), or 50 ng/ml RANKL (RANKL^{High}). Scale Bar represents 200 μm. FIG. 1B illustrates quantification of TRAP⁺ osteoclasts from experiments in FIG. 1A. Results were statistically significant with p<0.01 (**), and p<0.001 (***). Referring to these figures, it was observed that after 6 days of treatment with CM, TRAP staining revealed mature osteoclasts only in cells treated with CM from the highly bone metastatic 4T1.2 and TSU-Pr1-B2, although at significantly lower levels than treatment with 50 ng/ml RANKL (RANKL^{High}). FIG. 1C illustrates quantification of nuclei in TRAP⁺ osteoclasts from experiments in FIG. 1A. Results were statistically significant with p<0.05 (*), p<0.01 (**). Referring to FIGS. 1A to 1C, it was shown that combined treatment of RAW264.7 cells with CM and a limited level of RANKL (20 ng/ml, RANKL^{Low}) significantly increased differentiation, approaching that seen by treatment with 50 ng/ml of RANKL.

Treatment with CM induced similar differentiation in mouse bone marrow-derived primary pre-osteoclasts (FIG. 2C) and MOCP5 cells (FIGS. 2D to 2F).

FIG. 2C illustrates representative images of TRAP stained culture of primary bone marrow cells treated with 50 ng/ml M-CSF followed by 20 ng/mL RANKL and indicated CM. Scale bar represents 400 μm. FIG. 2D illustrates representative images of TRAP stained MOCP-5 cells treated with indicated CM for 6 days. Scale bar represents 400 μm. FIG. 2E illustrates quantification of TRAP⁺ osteoclasts treated with indicated conditions from experiment in FIG. 2D and shows that these results were statistically significant with p<0.05 (*), p<0.01 (**), p<0.001 (***). FIG. 2F illustrates quantification of nuclei in TRAP⁺ osteoclasts as in FIG. 2E. FIGS. 2G and 2H illustrate qRT-PCR mRNA expression levels of osteoclast genes in RAW264.7 cells under indicated treatments. Referring to these figures, the qRT-PCR results indicated that osteoclast genes were elevated in RAW264.7 cells upon treatment with RANKL and bone metastatic CM. Taken together, these results indicate that bone-metastatic breast cancer cells were capable of secreting factors that induce differentiation and maturation of osteoclasts.

### Example 3

### MiRNA Microarrays Reveal Differentially Regulated miRNAs

MiRNA microarray profiling was performed to compare miRNA expression changes in RAW264.7 cells treated for seven days with CM from 4T1 versus 4T1.2 cells or CM from TSU-Pr1 versus TSU-Pr1-B2 cells, and with or without 50 ng/ml RANKL. FIG. 1D illustrates heat map depicting miRNA microarray expression profiling in RAW264.7 cells treated with conditioned media from 4T1.2 or TSU-Pr1-B2 cells as compared to corresponding weakly metastatic cells 4T1 and TSU-Pr1, or 50 ng/ml RANKL as compared to non-treatment control. FIG. 1D, left panel, shows that among a total of 334 mouse miRNAs detected, 42 miRNAs were up-regulated and 45 miRNAs were down-regulated with a >2.2 fold change across treatment groups. FIG. 1D, right panel, shows 22 unique miRNAs consistently down-regulated across three sets of samples that were examined as an initial step toward identifying miRNAs that may inhibit osteoclast differentiation.

FIG. 1E illustrates correlation of miRNA expression changes in the indicated treatment pairs as in FIG. 1D. Referring to FIG. 1E, significant correlations of miRNA expression changes were observed between the treatment groups, indicating conservation in the miRNA regulatory network during osteoclastogenesis in physiological and pathological conditions. Genes known to be important for osteoclast differentiation and function were examined for predicted miRNA binding sites based on TargetScan (Grimson et al., 2007) and PicTar (Krek et al., 2005) predictions. See Example 5, Table 1. Five miRNAs, miR-33a-5p, miR-133a, miR-141-3p, miR-190, and miR-219-5p (hereinafter miR-33a, miR-141, and miR-219) were selected for further analysis based upon their significant down-regulation, multiple predicted osteoclast targets, and sequence conservation between human and mouse.

FIG. 1F illustrates qRT-PCR analysis of selected miRNAs in RAW264.7 or primary bone marrow derived osteoclasts after indicated length of treatment with 50 ng/ml RANKL. Data in the figure represent average ±SEM; p values were based on Student's t-test. The results were significant with p<0.05 (*), p<0.001 (***). Referring to FIG. 1F, the results from quantitative real-time PCR (qPCR) analysis confirmed that all five miRNAs examined decreased significantly after 6 days of RANKL treatment in RAW264.7, primary bone marrow-derived precursors and MOCP-5 cells. FIGS. 2A-2K also show osteoclast differentiation induced by RANKL and tumor conditioned media. These miRNA changes were also observed during the differentiation of primary human osteoclasts. FIG. 2I illustrates representative images of TRAP stained human osteoclasts treated with or without 33 ng/ml M-CSF and 66 ng/ml RANKL for 8 days. Scale bar represents 400 μm. FIGS. 2J and 2 K illustrate qRT-PCR miRNA (FIG. 2J) and mRNA expression levels (FIG. 2K) in osteoclasts from FIG. 2I. Data represent average ±SD. Data in the figure represent average ±SEM; p values were based on Student's t test. Result was statistically significant with p<0.05 (*).

### Example 4

### Ectopic Expression of miRNAs Inhibits Osteoclast Differentiation

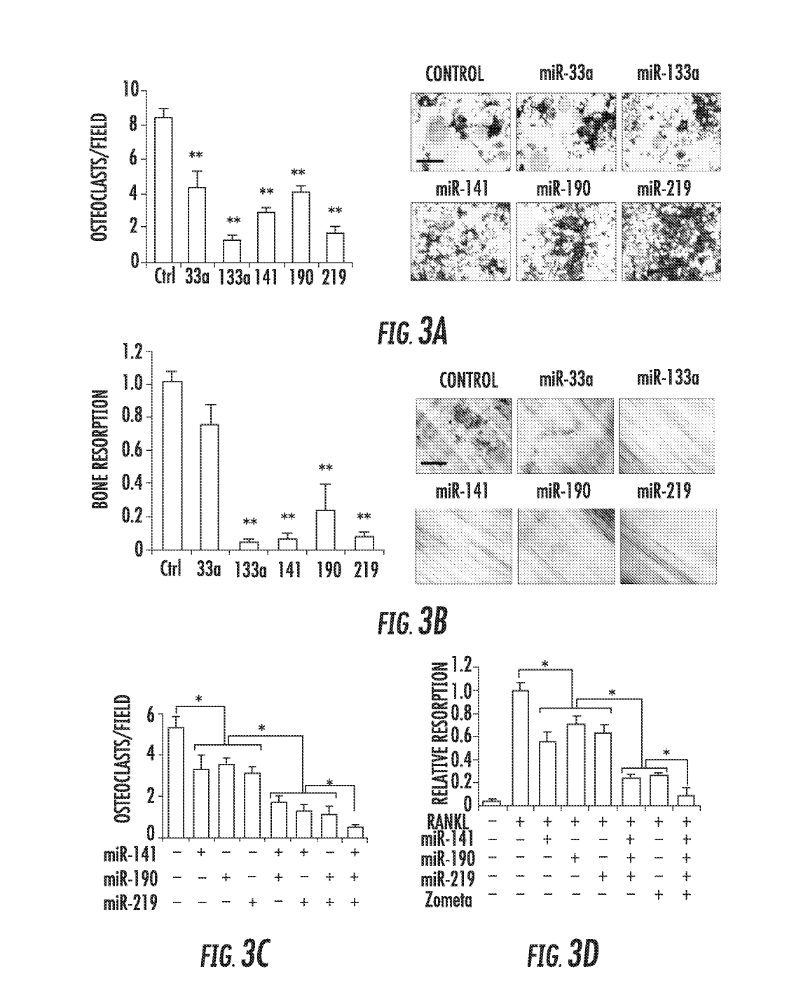

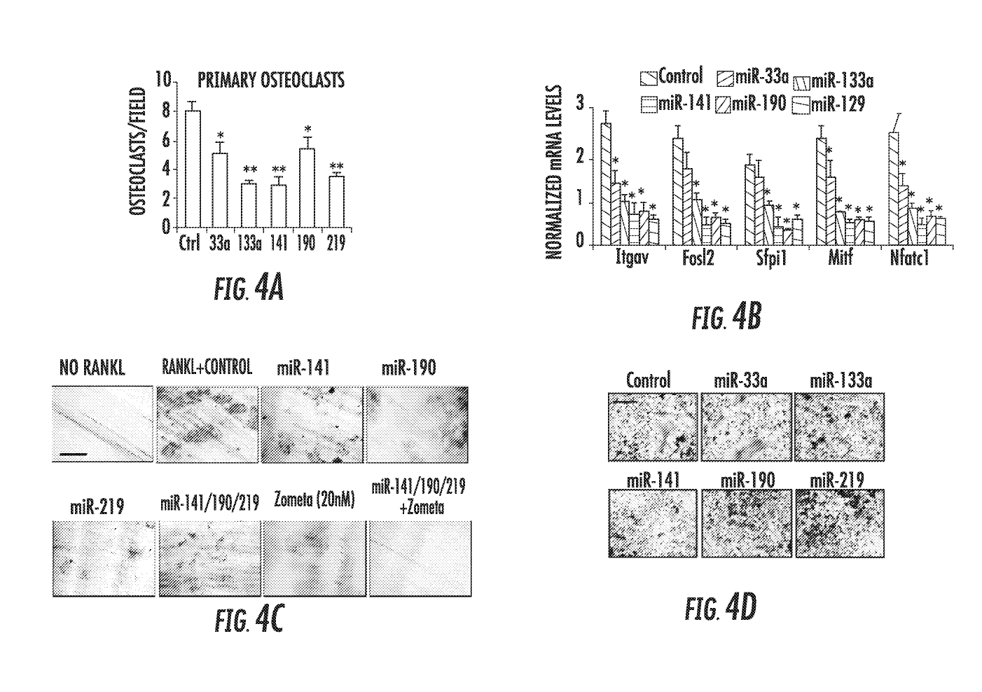

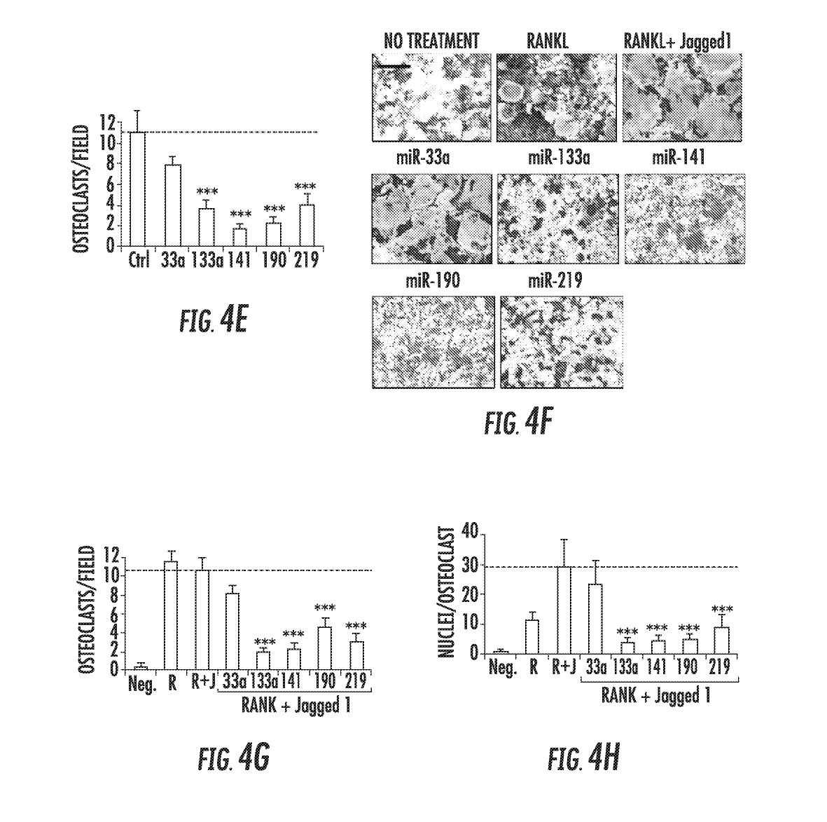

To examine the functional role of the down-regulated miRNAs in osteoclastogenesis, the miRNAs in pre-osteoclast cells were ectopically expressed prior to RANKL-induced osteoclast differentiation. FIG. 3A-3D illustrate ectopic expression of miRNAs downregulated during osteoclastogenesis inhibits osteoclast differentiation. FIG. 3A illustrates quantification and representative images of TRAP⁺ osteoclasts in RAW264.7 cells transfected with indicated miRNAs followed by treatment with 50 ng/ml RANKL. Scale bar represents 200 μm. Results were statistically significant with p<0.01 (**). FIG. 4A illustrates quantification of TRAP⁺ osteoclasts in primary bone marrow derived osteoclasts transfected with 1 pM of indicated miRNA oligonucleotide, followed by treatment with 50 ng/mL MCSF and 50 ng/mL RANKL. Data represent average ±SEM. p values were based on Student's t-test. Results were statistically significant with p<0.05 (*) and p<0.01 (**). Referring to FIG. 3A nd FIG. 4A, it was observed, that overexpression of all five miRNAs resulted in a significant decrease in osteoclast differentiation in both RAW264.7 cells and primary pre-osteoclasts. No change was observed in cell growth or apoptosis after any of the miRNA treatments. The defect in osteoclastogenesis was confirmed by qRT-PCR analysis that found repression of multiple osteoclast marker genes after ectopic miRNA expression. FIG. 4B illustrates RT-PCR mRNA expression levels of Itgav, Fosl2, Sfpi1, Mitf and Nfatc1 genes in RAW264.7 cells transfected with miRNAs, followed by treatment with 50 ng/ml RANKL. The results were significant with p<0.05 (*).

In vitro bone resorption assay was then used to measure the effect of ectopic miRNA expression on osteoclast activity. FIG. 3B illustrates quantification and representative images of bone resorption by RAW264.7 cells after treatment in conditions as shown in FIG. 3A, relative to control cells. Referring to this figures, scale bar represents 200 μm. Results were statistically significant with p<0.01 (**) and p<0.001 (***). Referring to this figure, it was observed that while miR-33a caused only a small decrease in bone resorption, the remaining four miRNAs, miR-133a, miR-141, miR-190 and miR-219, dramatically limited osteoclast activity. Since these miRNAs may inhibit osteoclast differentiation or function by targeting different mRNAs, the repression of osteoclast differentiation was further examined using combinations of inhibitory miRNAs. Given that transient transfection with 10 pM of miRNA precursors was sufficient to inhibit up to approximately 90% of osteoclast differentiation, RAW264.7 cells were treated with a combination of miRNAs at a reduced concentration of 1 pM total precursors.

FIG. 3C illustrates quantification of TRAP⁺ osteoclasts from RAW264.7 treated cells transfected with 1 pM indicated miRNAs, followed by treatment with 20 ng/ml RANKL. Results were statistically significant with p<0.05 (*). FIG. 3D illustrates quantification of relative bone resorption in RAW264.7 cells transfected with 1 pM indicated miRNA or 20 nM Zometa. Data in the figure represent average ±SEM. p values were based on Student's t-test. Results were significant with p<0.05 (*). FIGS. 4A-4H illustrate inhibition of osteoclast differentiation by ectopic expression of miRNAs downregulated during osteoclastogenesis. FIG. 4C illustrates representative images of bone resorption in RAW264.7 cells transfected with indicated miRNAs or treated with 20 nM Zometa. Scale bar represents 200 μm. Referring to FIG. 3C, FIG. 3D and FIG. 4C, it was observed that combined ectopic expression of miR-141, miR-190, and miR-219 revealed a significant additive effect on osteoclast maturation (FIG. 3C) and bone resorption (FIG. 3D and FIG. 4C). Referring to FIG. 3D and FIG. 4C, it was observed that this addititive effect was equivalent to the treatment with 20 nM zoledronic acid. Ectopic expression of the miRNAs combined with zoledronic acid revealed an additive effect, leading to almost complete ablation of osteoclast activity. These results show that repression of multiple miRNAs during osteoclast differentiation, with ectopic expression of miR-133a, miR-141, and miR-219 strongly inhibit osteoclast maturation and activity in all models tested. Partial inhibition was observed after ectopic expression of miR-33a and miR-190.

To confirm that miRNA mediated inhibition of osteoclast differentiation is not specific to the RAW264.7 model, the effect of ectopic miRNA expression was examined in additional models. FIG. 4D illustrates representative images of TRAP⁺ osteoclasts from MOCP-5 cells transfected with indicated miRNAs, followed by treatment with 50 ng/mL RANKL. Scale bar represents 200 μm. FIG. 4E illustrates quantification of TRAP⁺ osteoclasts from experiment shown in FIG. 4D. The results were statistically significant with p<0.001 (**). Referring to FIGS. 4D and 4E, it was observed that MOCP5 cells transfected with pre-miRNAs revealed a similar pattern of inhibition as was observed for RAW264.7 cells.

To examine the effect of miRNAs on Jagged1-enhanced osteoclast differentiation after activation of the Notch pathway, RAW264.7 cells were plated on Jagged1 coated plates, followed by treatment with 50 ng/ml RANKL. FIG. 4F illustrates representative images of RAW264.7 cells treated with miR-33a, miR-133a, miR-141, miR-190 and miR-190 and grown on Jagged1 coated plates, followed by treatment with 50 ng/mL RANKL. Scale bar represents 200 μm. FIG. 4 G illustrates quantification of TRAP⁺ osteoclasts from experiment in FIG. 4F. FIG. 4H illustrates quantification of nuclei per osteoclast from experiment in FIG. 4F. Data in the figure represent average ±SEM; p values were based on Student's t test. Results were significant with p<0.001 (***). Referring to FIGS. 4F to 4H, it was observed that ectopic miRNA expression significantly inhibited Jagged1-dependent enhancement of osteoclast differentiation. Taken together, these results illustrate the broad capacity for ectopic expression of these miRNAs to inhibit osteoclast differentiation.

### Example 5

### Identification of miRNA Targets Involved in Osteoclast Differentiation and Function

Direct miRNA targeting of mRNAs related to osteoclastogenesis was examined using luciferase reporters containing the 3′UTR of prospective target mRNAs (Table 1). miR-33a, miR-133a, miR-141, miR-190 and miR-219 are indicated by bold characters.

TABLE 1
Predicted miRNA target genes and fold change of their
expression as measured by microarray in RAW264.7 cells
treated with RANKL or CM.

| | | Microarray | |
|---|---|---|---|
| | | fold | Conservation |
| miR-RNA | Predicted target genes | change | (hsa & mmu) |
| miR-9 | Mmp14 | −2.450 | + |
| miR-32 | Acp5 | −3.262 | + |
| miR-33a | Calcr, Cpr | −2.445 | + |
| miR-133a | Mmp9, Mmp14, Itgav, | −2.885 | + |
| | Oscar, Mitf | | |
| miR-139 | NFATc1 | −3.321 | + |
| miR-141 | Acp5, Calcr, Itgav, Mitf, | −3.511 | + |
| | Mmp9 | | |
| miR-186 | Itgav | −2.390 | + |
| miR-190 | Calcr, Nfatc1 | −3.655 | + |
| miR-193 | Mmp14 | −2.317 | + |
| miR-219 | Ctsk, Calcr, Mitf, Traf6 | −2.673 | + |
| miR-296 | Mmp9 | −2.243 | + |
| miR-369-5p | Calcr, Cpe | −2.334 | + |
| miR-376c | Ctsk, Calcr, Itgav | −2.588 | + |
| miR-520a | Rank, Calcr | −2.483 | − |
| miR-545 | Acp5, Calcr, Itgav | −2.785 | − |
| miR-586 | Itgav, Cpe | −2.742 | − |
| miR-611 | Nfatc1 | −2.501 | − |
| miR-624 | Mst1r, Itgav | −2.230 | − |
| miR-651 | Acp5, Rank, Itgav | −2.541 | − |

Reporter constructs were co-transfected with pre-miRs, as well as a Renilla luciferase plasmid for normalization.

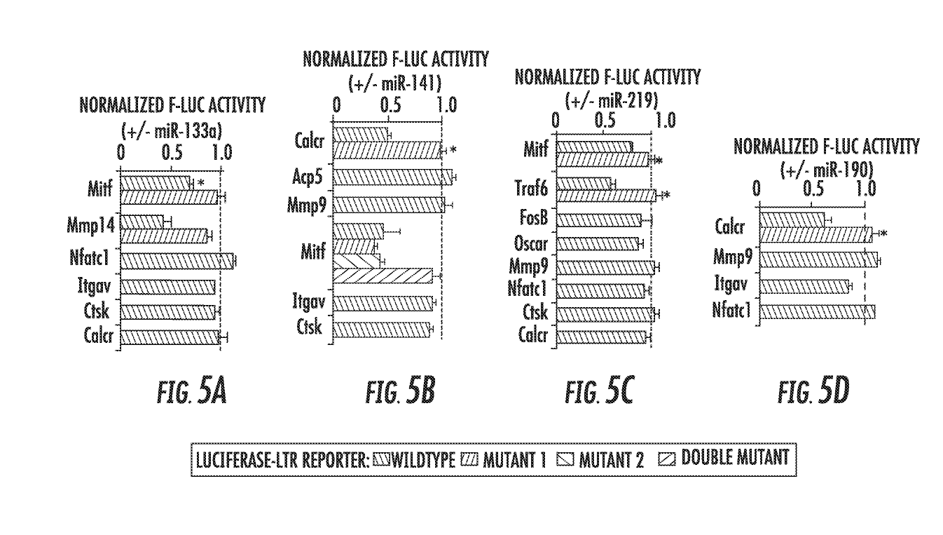

FIGS. 5A-5E illustrate validation of candidate miRNA genes. FIGS. 5A-5D illustrate normalized activity of luciferase reporter containing the 3′ UTR of candidate genes upon co-transfection with indicated miRNAs, relative to transfection with negative control miRNA. The results were statistically significant with p<0.05 (*). The following target genes were tested: Mitf, Mmp14, Nfatc1, Itgav, Ctsk, Calcr, Acp5, Mmp9, Traf6, FosB, and Oscar. In the drawings, light gray bars indicate wildtype constructs, dark gray bars indicate mutant 1, dotted bars indicate mutant 2 and diagonally stripped bars indicate double mutant. Reporter assays revealed a number of direct targets for the candidate miRNAs. Referring to FIGS. 5A-5C, it was observed that miR-133a, miR-141, and miR-219 were all found to repress expression of Mitf. Specific targeting of Mitf by these miRNAs was confirmed by site-directed mutagenesis of the predicted miRNA binding sites. Table 2 shows alignment of miRNA sequences with sequences of the target genes.

TABLE 2
MiRNA alignment to 3′UTR of target genes.

| miRNA and | |
|---|---|
| target mRNA | Sequence alignment |
| miR-133a | 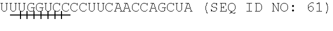 |
| MMP14 | AAACCAGGGGAGGACGGGGGAGA (SEQ ID NO: 62) |
| MITF | AAACCAGUGAGGUAUAUCUCCG (SEQ ID NO: 63) |
| miR-190 | 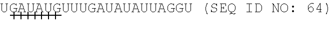 |
| CALCR | UACUAUACCAGUCGAGGACCCUC (SEQ ID NO: 65) |
| miR-219 | 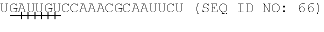 |
| MITF | UACUAACAUAGCCAGUUGUGAUA (SEQ ID NO: 67) |
| TRAF6 | GACUAACAUACACACGUAGAGGA (SEQ ID NO: 68) |
| miR-141 | 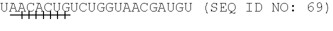 |
| CALCR site1 | AUUGUGACACUAACUUUUUUGUA (SEQ ID NO: 70) |
| CALCR site2 | AUUGUGAAGAAGUGUUUACUAU (SEQ ID NO: 71) |
| MITF site1 | UUUGUGACUUUUUUUACUACUAU (SEQ ID NO: 72) |
| MITF site2 | UUUGUGACGCAGAACCGAAUUGG (SEQ ID NO: 73) |

Referring to FIG. 5B and FIG. 5C, miR-141 was found to contain two active binding sites in the Mitf 3′UTR, reducing expression by ˜50% (FIG. 5B), while a single site in miR-219 was able to repress Mitf expression by ˜20% (FIG. 5C). Referring to FIGS. 5A-5D, additionally it was found that miR-133a repressed Mmp14, miR-141 repressed Calcr, miR-219 repressed Traf6, and miR-190 was able to repress Calcr.

These miRNA targets represent important factors involved in osteoclast differentiation and function, with Mitf functioning as an essential transcription factor, Calcr and Traf6 serving as important signal transducers, and Mmp14 functioning as a secreted matrix metalloproteinase during osteoclastogenesis (FIG. 5E). FIG. 5E illustrates schematically miRNA targets that are involved in osteoclast differentiation or function. Referring to this figure, it was found that miR-133a, miR-219 and miR-141 target Mitf. It was found that miR-219 also targets Traf6, miR-133a also targets Mmp14, and MiR-190 and miR-141 also target Calcr.

To better examine the capacity of the miRNAs to inhibit osteoclast differentiation, osteoclast markers were examined in RAW264.7 cells transfected with the miRNA of interest, followed by RANKL treatment. FIG. 5F illustrates Western blot analysis for Pu.1, Nfatc1, or Ctsk in RAW264.7 cells after transient transfection with indicated miRNA and treatment with 50 ng/ml RANKL. Data in the figure represent average ±SEM; p values were based on Student's t-test. Referring to FIG. 5F, Western blot analysis of Pu.1, Nfatc1, and Ctsk revealed that miR-133a, miR-141, and miR-219 inhibited early osteoclast differentiation, with minimal expression of Nfatc1, which plays a role in early osteoclast commitment, or the mature osteoclast marker Ctsk (Teitelbaum and Ross, 2003). These results are in agreement with data on targeting of Mitf or Traf6, either of which is predicted to inhibit the earliest stages of osteoclast commitment. In comparison, miR-190 transfected cells expressed Pu.1 and Nfatc1, but not Ctsk, indicating that miR-190 might be inhibiting osteoclast differentiation after commitment to an osteoclast fate. It was also observed that miR-33a transfected cells retained expression of all three osteoclast genes, consistent with its relatively weak effect on inhibiting osteoclastogenesis. Taken together, these results indicate that the miRNAs inhibited osteoclast differentiation through the functional targeting of essential osteoclast genes functioning at different stages of osteoclast differentiation.

### Example 6

### Systemic miRNA Treatment Inhibits Osteoclasts In Vivo

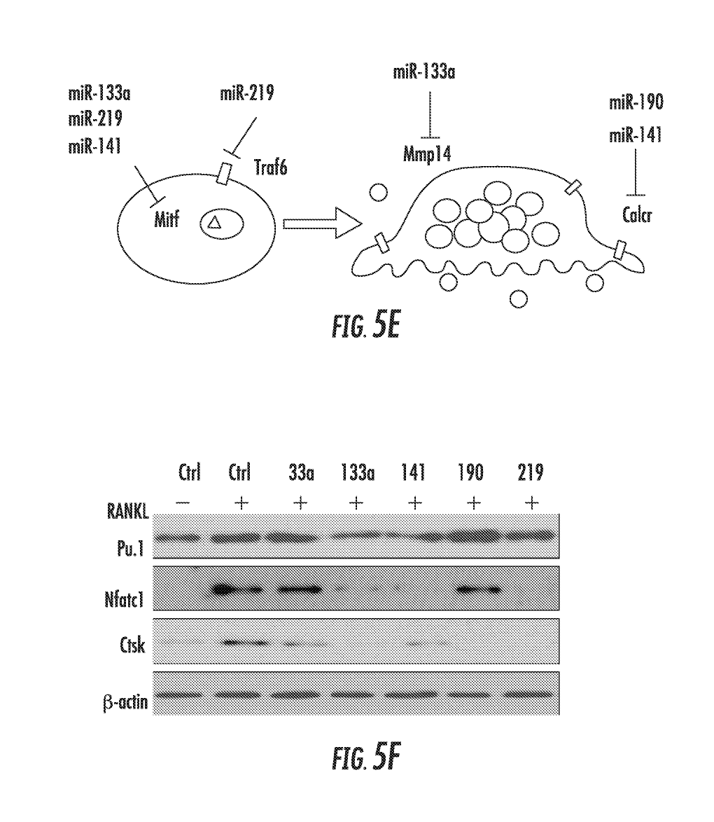

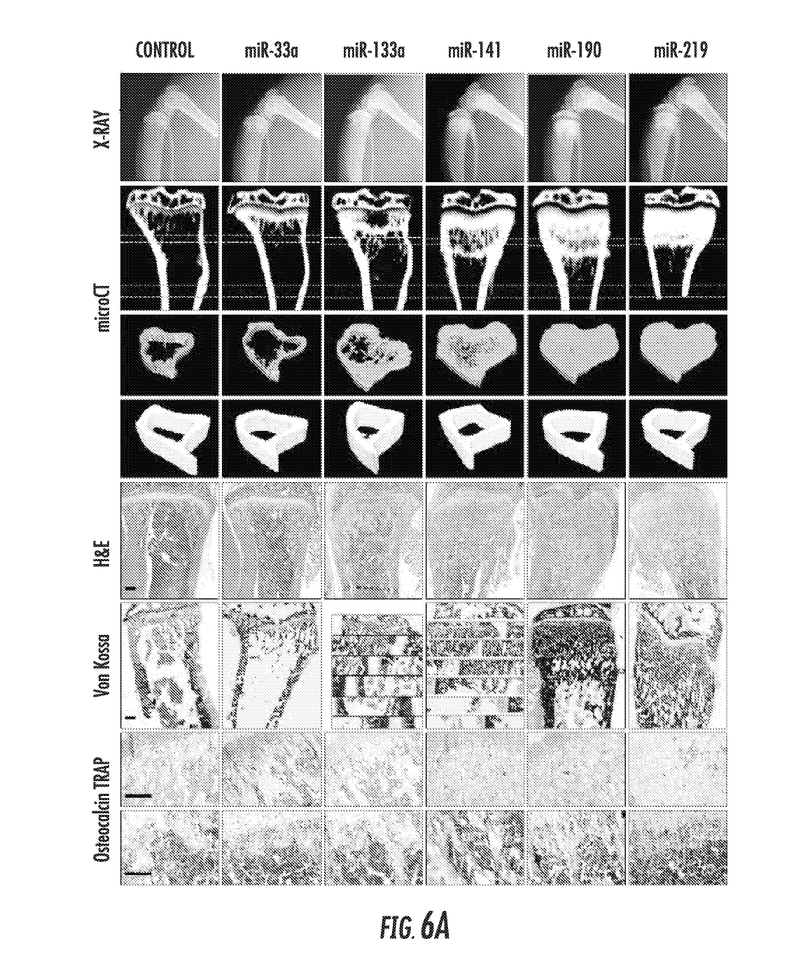

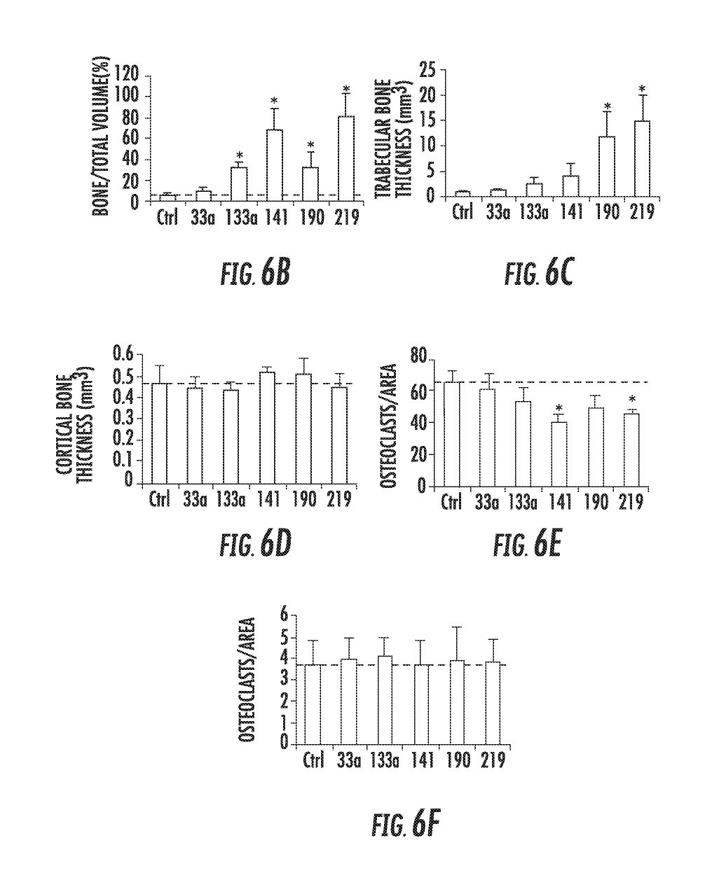

To evaluate the capacity for these miRNAs to inhibit mouse osteoclast function in vivo, 10 μg of pre-miRNA were injected into the lateral tail-vein of Balb/c mice weekly for four weeks. Mice were examined weekly by X-ray radiography, revealing increased bone density in the hind limbs, particularly in the distal femur and proximal tibia. FIGS. 6A-6F illustrate micro-computed tomography and histological analysis, showing alterations in bone homeostasis after treatment with miRNAs downregulated in osteoclastogenesis. FIG. 6A illustrates representative X-ray, μCT and histological images for TRAP, H&E, Von Kossa, or osteocalcin staining of bones from mice treated with miR-33a, miR-133a, miR-141, miR-190 and miR-219 compared to non-treated control. Regions of interest for trabecular and cortical bone scan and analysis are marked by dashed lines. Scale bar indicates 200 μm, n=6. Referring to FIG. 6A, examination by microCT revealed increased trabecular bone in both femur and tibia. FIG. 6B illustrates quantification of bone volume relative to total volume from representative μCT scans in FIG. 6A. n=3. Results were statistically significant with p<0.05 (*). FIG. 6C illustrates quantification of trabecular thickness from representative μCT scans of mice treated with miRNAs as in FIG. 6B. Results were statistically significant with p<0.05 (*). FIG. 6D illustrates quantification of cortical bone thickness from representative μCT scans as in FIG. 6B. FIG. 6E illustrates quantification of TRAP⁺ osteoclasts from decalcificed histological sections of hindlinbs from mice in FIG. 6A, n=6. Results were statistically significant with p<0.05 (*). FIG. 6F illustrates Quantification of osteocalsin-positive osteoblasts from bone sections from mice in FIG. 6A. Data in the figure represent average ±SEM; p values were based on Student's t-test. Referring to FIGS. 6B, 6C and 6D, quantitative analyses confirmed a significant increase in bone volume (FIG. 6B) and trabecular thickness (FIG. 6C), while no significant differences in cortical bone thickness was found (FIGS. 6A and 6D). Referring to FIGS. 6A and 6E, histological TRAP staining of decalcified bone sections revealed a significant decrease in the number of osteoclasts relative to bone surface area in mice treated with miR-141 and miR-219 (FIGS. 6A and 6E) and Von Kossa staining revealed the increase of calcified tissues (FIG. 6A), confirming the significant expansion in trabecular bone that was seen in the microCT analysis. Referring to FIGS. 6A and 6F, osteocalcin immunohistochemical staining revealed no significant difference in osteoblast number, consistent with a previous report that inhibition of miRNA biogenesis does not affect osteoblast differentiation (Mizoguchi et al., 2010). While these results indicated osteoclasts as a major bone cell type affected by systemic delivery of these miRNAs, it is important to note that the differentiation or activity of additional bone stromal cells might also be impacted.

### Example 7

### MiR-141 and miR-219 Inhibit Experimental Bone Metastasis

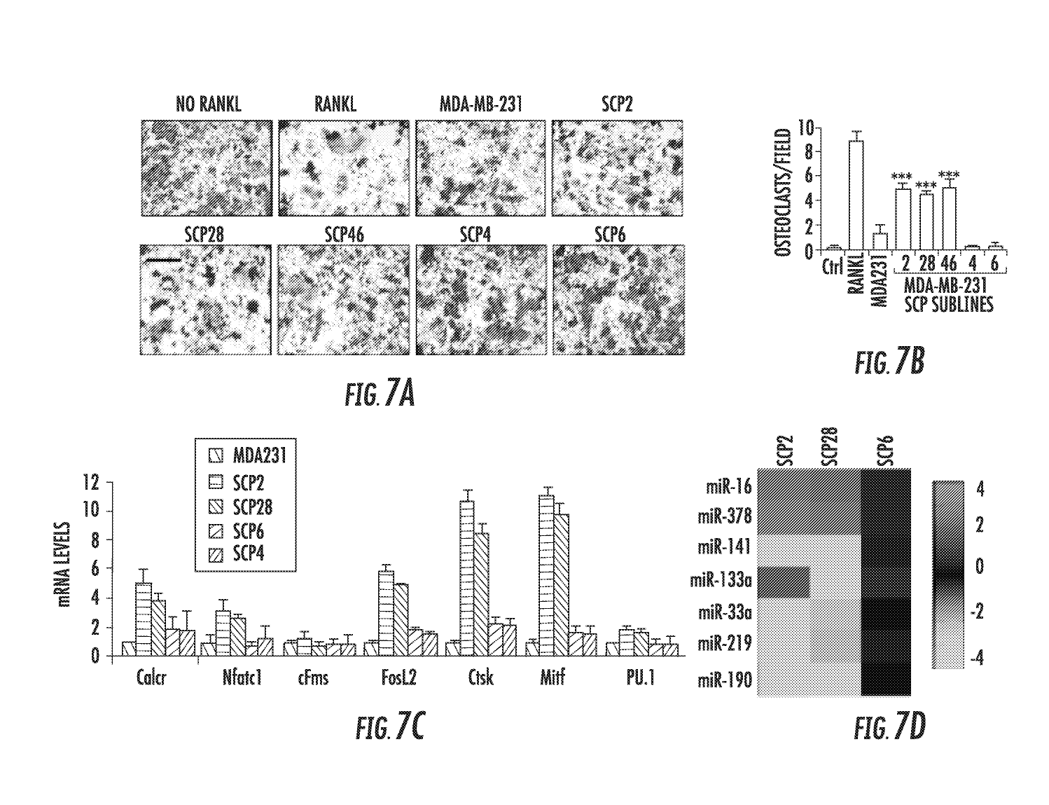

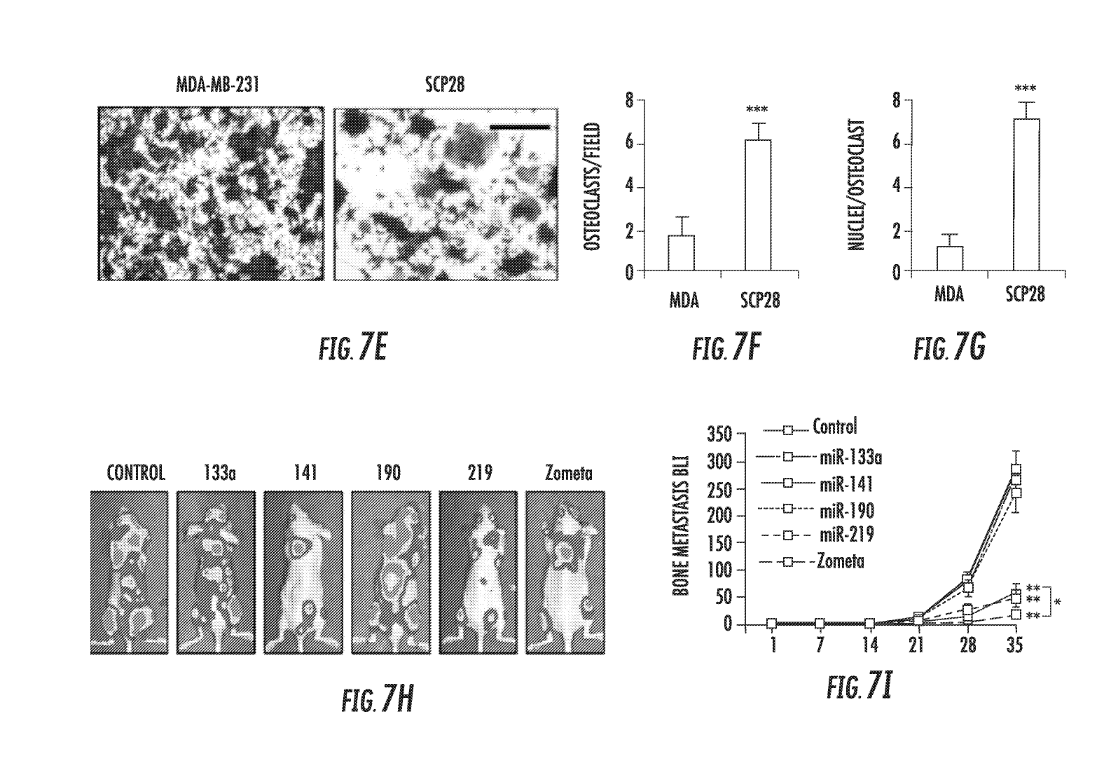

Given the ability of multiple miRNAs to inhibit osteoclast differentiation in vitro and in vivo, the capacity of these miRNAs to inhibit breast cancer bone metastasis was next examined in a mouse model. See Kang et al., 2003 and Blanco et al., 2012. The potential of CM from multiple SCP cell lines, clonal derivatives of the human MDA-MB-231 breast cancer line with different bone metastatic capabilities, to regulate osteoclast differentiation was examined first. FIGS. 7A-7I illustrate that conditioned media of bone-tropic sublines of MDA-MD-231 induces osteoclast differentiation. FIG. 7A illustrates representative images of RAW264.7 cells treated with 50 ng/ml RANKL or indicated CM (MDA-MB-231). Scale bar represents 200 μm. FIG. 7B illustrates quantification of osteoclast differentiation from RAW264.7 cells in experiment in FIG. 7A. The results were statistically significant with p<0.001 (***) as assessed by Student's t test. FIG. 7C illustrates qRT-PCR mRNA expression levels of Calcr, Nfatc1, cFms, FosL2, Ctsk, Mitf, and PU.1 genes in RAW264.7 cells treated with CM from MDA231. SCP2, SCP28, SCP6, SCP4 cell lines. FIG. 7D illustrates heat map depicting qRT-PCR miRNA expression profiling in RAW264.7 cells after treatment with CM from the indicated cell lines relative to the treatment with CM from the MDA-MB-231 parental cell line.

Referring to FIGS. 7A-7D, it was observed that CM from highly bone metastatic SCP cell lines (SCP2, 28 and 46), but not from weakly metastatic lines (SCP4, SCP6 and parental MDA-MB-231), stimulated osteoclast differentiation in RAW264.7 cells (FIGS. 7A and 7B), increased the expression of osteoclast marker genes (FIG. 7C), and induced miRNA expression changes consistent with our previous findings (FIG. 7D).

After further validating the ability of highly metastatic SCP28 to induce osteoclastogenesis in MOCP5 cells, SCP28 were used for analyzing the effect of miRNAs on bone metastasis development in mice. FIG. 7E illustrates representative images of MOCP-5 cells treated with MDA-MB-231 and SCP28 CMs. Scale bar represents 200 μm. FIGS. 7F and 7G illustrate quantification of osteoclast number and nuclei per osteoclast after MOCP-5 cells were treated with indicated CM as in FIG. 7C. Results were statistically significant with p<0.001 (***) as assessed by Student's t test. Nude mice were systemically treated with control or experimental pre-miRNAs immediately before, and once per week subsequently, after inoculation with SCP28 cells via intracardiac injection. Metastatic progression was monitored by weekly bioluminescence imaging (BLI) using a firefly luciferase reporter stably expressed in the cell line.

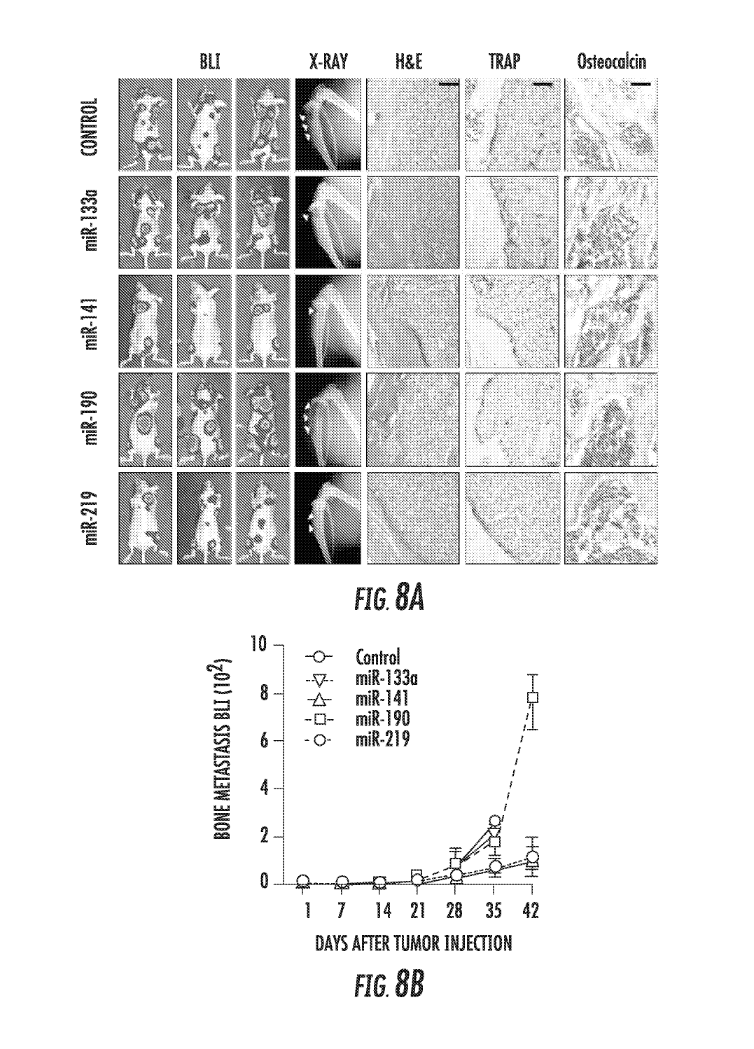

FIGS. 8A-8F illustrate systemic treatment with miRNAs downregulated in osteoclastogenesis, miR-133a, miR-141, miR-190, and miR-219, inhibits bone metastasis. FIG. 8A illustrates BLI, X-ray, Osteocalcin, H&E, and TRAP images from mice inoculated with SCP28 breast cancer cells and treated with indicated miRNAs. BLI images show three representative mice from each experimental group. White arrows indicate osteolytic bone lesions in the X-ray images. Scale bar represents 200 μm. n=10. FIG. 8B illustrates normalized bone metastasis BLI signals from mice in FIG. 8A. n=10. The results were statistically significant with p<0.01 (**) assessed by Mann-Whitney test. Referring to FIGS. 8A and 8B, it was observed that whereas treatment with miR-133a and miR-190 had no effect on metastatic progression by BLI, there was a significant decrease in hind-limb tumor burden after treatment with either miR-141 (p=0.024) or miR-219 (p=0.015).

FIG. 8C illustrates qRT-PCR miRNA expression levels from serum samples of mice taken at indicated times after injection with miRNAs. The results were statistically significant with p<0.01 (**). Referring to this figure, qPCR analysis of serum samples from mice inoculated with the pre-miRNAs revealed a substantial decrease in miR-190 and miR-133a within 6 hours of injection, with nearly full clearance within 24 hours. In contrast, miR-141 and miR-219 took substantially longer to clear from the serum, maintaining ˜20% of the normalized expression at 24 hours.

FIG. 8D illustrates quantification of osteolytic lesion area in hindlimbs from indicated experimental group. The results were statistically significant with p<0.01 (**) and n=6. Referring to FIGS. 8A and 8D, X-ray imaging revealed decreased bone lesions in miR-141 and miR-219 treated mice while miR-190, miR-133a and control pre-miRNA injected mice exhibited significant bone degradation. Histological analysis revealed a decrease in osteolysis by hematoxylin and eosin (H&E) staining and a decrease in TRAP⁺ osteoclast recruitment at the tumor-bone interface.

FIG. 8E illustrates quantification of TRAP⁺ osteoclasts from histology in experiment from FIG. 8A. The results were statistically significant with p<0.01 (**). FIG. 8F illustrates quantification of osteocalcin⁺ osteoblasts from histology in experiment from FIG. 8A. Data in the figure represent average ±SEM; p values were based on Student's t-test unless otherwise indicated.

Referring to FIGS. 8A, 8E, 8F and 6A-6E, it was observed that consistent with the results in miRNA-treated healthy mice (FIGS. 6A-6E), miR-190, miR-141, and miR-219 treated mice showed a significant decrease in TRAP⁺ osteoclast number (FIG. 8E), while there was no noticeable change in osteoblast number from any of the treatments (FIGS. 8A and 8F). To rule out the potential influence of the miRNAs on the growth or survival of the SCP28 cells, the cells with miRNAs were treated in culture, and no significant difference in proliferation was found. Taken together, these results reveal a significant decrease in metastatic burden after systemic treatment with miR-141 or miR-219, likely due to decreased osteoclast activity.

The therapeutic effect of miRNAs was further compared with treatment of 100 μg/kg zoledronic acid (ZOMETA™). FIG. 7H illustrates representative BLI images of mice after inoculation with SCP28 cells and treatment with miR-133a, miR-141, miR-190, miR-219, or 100 μg/kg ZOMETA™ compared to no-treatment control, n=10. FIG. 7I illustrates normalized bone metastasis BLI signals from mice in FIG. 6H. Data in the figure represent average ±SEM. The results were statistically significant with p<0.001 (***) as was assessed by Mann-Whitney test when compared with control treatment group. Referring to FIGS. 7H and 7I, mice treated with ZOMETA™ experienced a significant decrease in tumor burden, with an overall trend of better response than individual miR-141 or miR-219 treatment, although the difference did not reach statistical significance. These results indicate that systemic treatment with miR-141 and miR-219 inhibits bone metastasis burden to a similar extent as current therapeutics.

### Example 7

### Serum miRNA Levels Correlate with Bone Metastasis

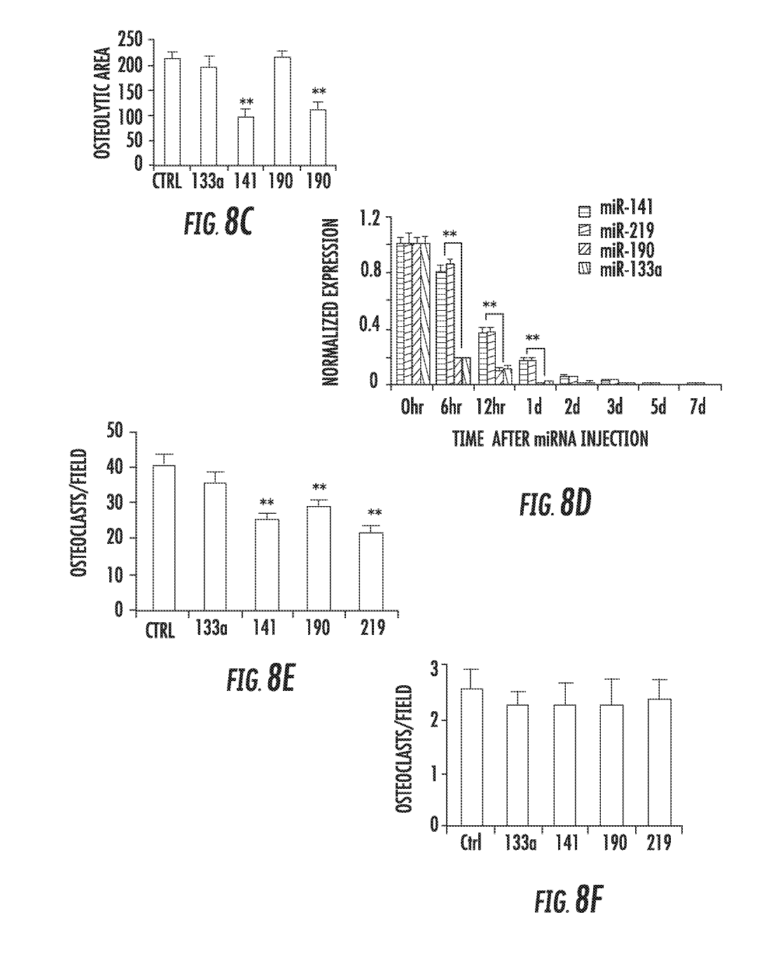

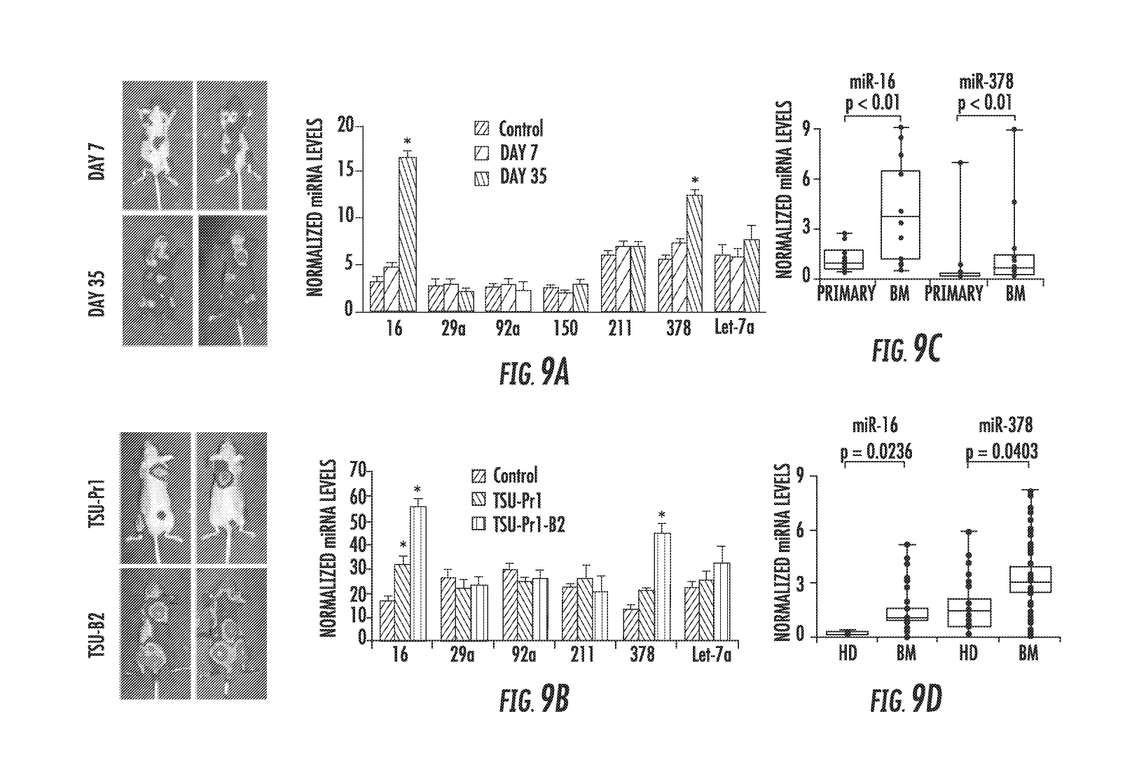

To further evaluate the function of miRNAs that change during osteoclast differentiation, a subset of four miRNAs with significant up-regulation during osteoclastogenesis (miR-16, miR-211, miR-378 and Let-7a, FIG. 1D) was examined next. No significant differences were observed after ectopic expression or repression of any of the candidate miRNAs tested. However, additional candidate miRNAs may be tested. As miRNAs are often released from cells into circulation, miRNAs with increased expression during osteoclastogenesis may potentially serve as biomarkers for osteolytic bone metastasis. To evaluate the possibility of using osteoclast miRNAs as biomarkers, the expression of miRNAs up-regulated during osteoclastogenesis (FIG. 1D) was examined in serum samples of mice with different bone metastasis burdens. Serum samples were collected at 0, 7, and 35 days from nude mice after intracardiac injection of highly metastatic SCP2 cells and miRNA expression was analyzed by qRT-PCR. FIGS. 9A-9D illustrate that serum levels of miRNAs upregulated during osteoclastogenesis, miR-16 and miR-378, correlate with bone metastasis.

FIG. 9 A illustrates BLI and qRT-PCR expression analysis of miRNAs in mice after intravenous inoculation with SCP2 breast cancer cells. Left, BLI images of two representative mice at each time point. Right, qRT-PCR for miRNA expression levels of miRNAs derived from serum samples taken on indicated day post-injection. Light gray bar indicates control, dark gray bar indicate the miRNA level on day 7, diagonally stripped bar indicates the miRNA level on day 35. Data represent average ±SEM. n=10. The results were statistically significant with p<0.05 (*) assessed by Student's t-test. Referring to this figure, examination of multiple miRNAs with the most dramatic increase during differentiation revealed consistently elevated expression of miR-378 and miR-16 at 35 days post-injection.

FIG. 9B illustrates BLI and qRT-PCR expression analysis of miRNAs in mice 28 days after intravenous inoculation with TSU-Pr1 (dark gray) or TSU-Pr1- (diagonally stripped) bladder cancer cells compared to control (light gray). Data represent average ±SEM. The results weres statistically significant with p<0.05 (*), n=10 assessed by Student's t-test. Referring to FIG. 9B, it was observed that only miR-378 and miR-16 were elevated when mice with low of high bone metastatic tumor burden after inoculation with weakly metastatic TSU-Pr1 or highly metastatic TSU-Pr1-B2 cell lines, respectively, were compared. Based on these findings, the expression of miR-378 and miR-16 was examined in matched primary (Primary) or bone-metastatic (BM) tumor samples from 12 breast cancer patients.

FIG. 9C illustrates qRT-PCR miRNA expression analysis of matched micro-dissected primary breast tumor (Primary) or bone metastasis (BM) samples. p values based on Mann-Whitney test, n=12. Referring to FIG. 9C, it was observed that both miR-16 and miR-378 have elevated expression in bone metastases, reflecting the presence of osteoclasts. Next, the expression of these two miRNAs was analyzed in serum samples of healthy female donors (HD) or breast cancer patients with bone metastasis (BM). FIG. 9D illustrates qRT-PCR miRNA expression analysis of serum samples from healthy donors (HD) or breast cancer patients with bone metastasis (BM). p values based on Mann-Whitney test and HD, n=21, BM, n=38. Referring to FIG. 9D, it was observed significantly increased level of both miR-16 and miR-378 in patients with bone metastasis. Taken together, these results indicated the potential for using these miRNAs as biomarkers for bone metastasis progression.

### Example 8

### Soluble ICAM1 from Metastatic Cells Enhances Osteoclast Differentiation

Since tumor CM induced osteoclastogenesis produced similar miRNA changes to RANKL-induced physiological osteoclast differentiation, soluble factor(s) in CM that promote osteoclast activation were identified. First, RANKL present in CM was investigated to cause such activities.

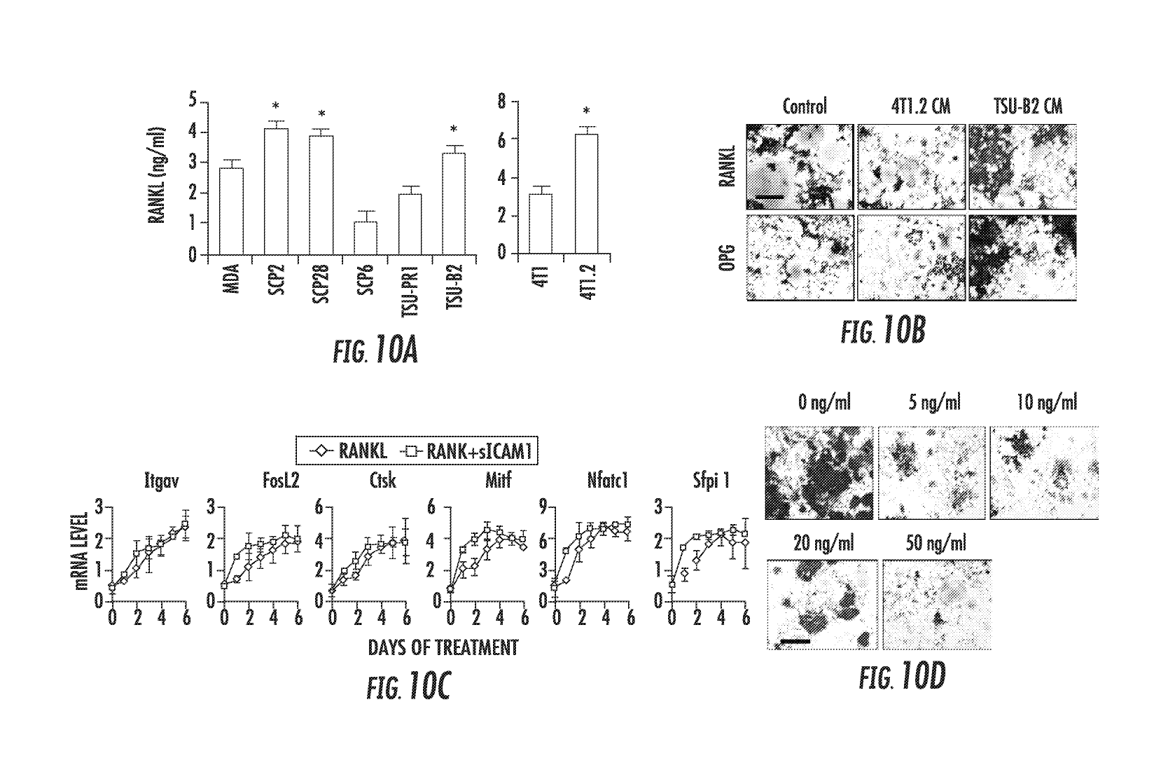

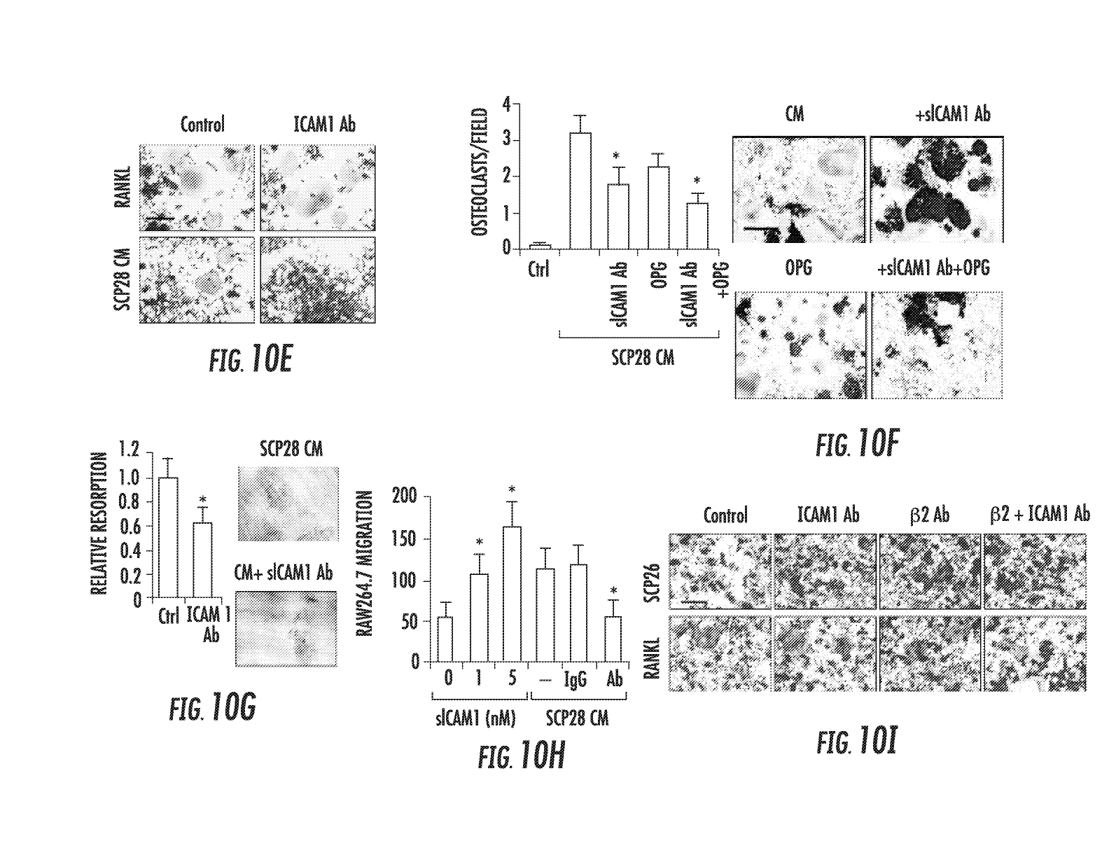

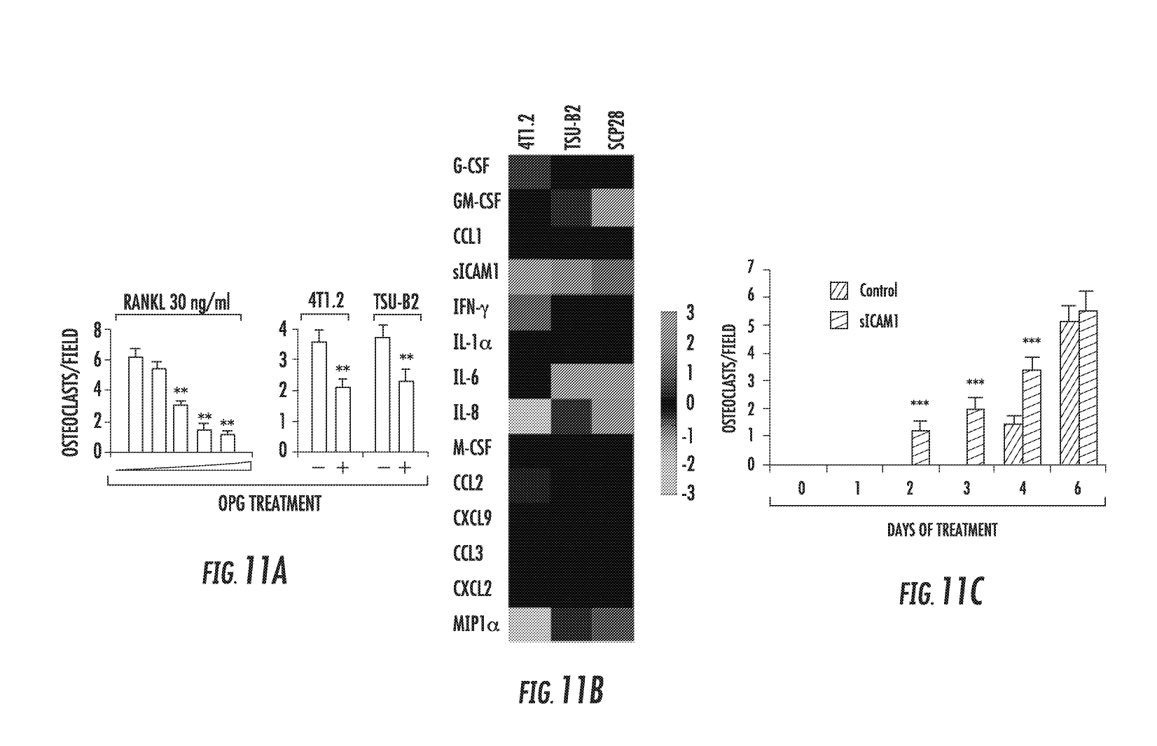

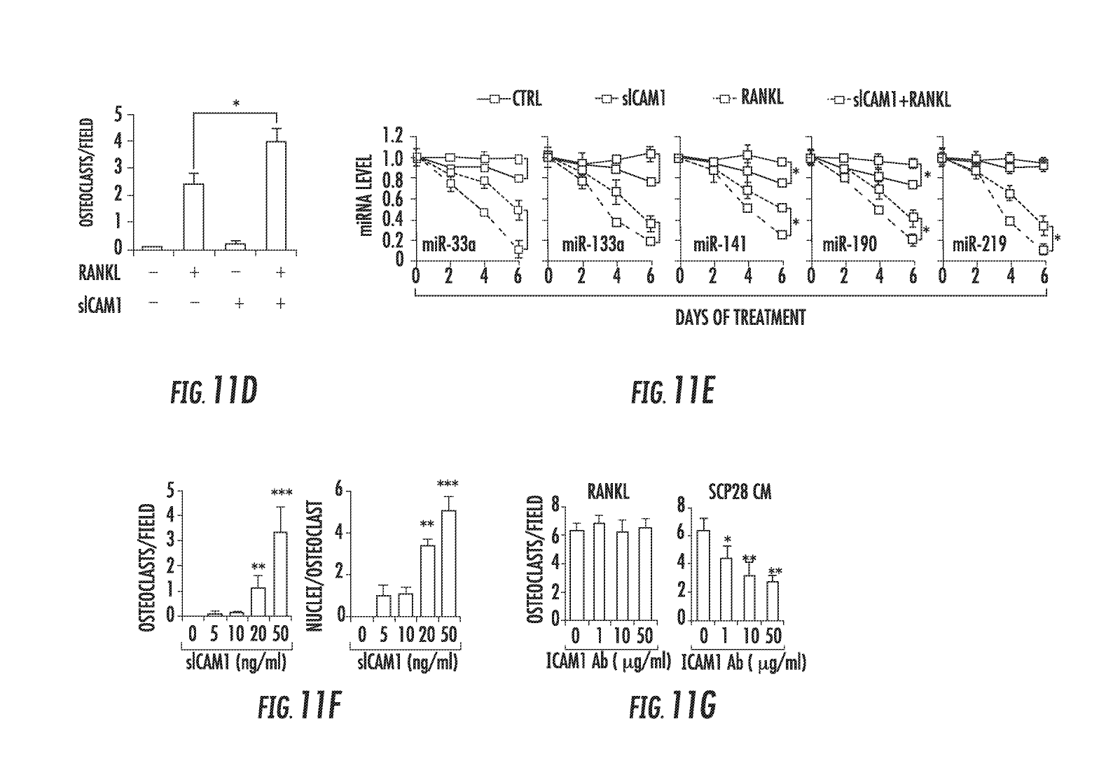

FIGS. 10A-10I illustrate that sICAM1 mediated osteoclast differentiation is β2 integrin dependent. FIG. 10A illustrates RANKL protein level in MDA, SCP2, SCP28, SCP6, TSU-PR1, TSU-B2, 4T1 and 4T1.2 CM samples as determined by ELISA. The results were statistically significant with p<0.05 (*). Referring to this figure, ELISA quantification of RANKL levels in CM samples revealed very low levels (4-6 ng/ml) of RANKL even in highly metastatic MDA-MB-231, 4T1 or TSU sublines, although these sublines produced significantly higher levels of RANKL than their weakly metastatic isogenic counterparts. FIG. 10B illustrates representative images of RAW264.7 cells treated with RANKL or the indicated CM±200 ng/mL OPG as in FIG. 10A. Scale bar represents 400 μm. Referring to FIGS. 10A and 10B, it was observed the low levels of 4-6 ng/ml levels of RANKL were insufficient to induce osteoclast differentiation. Furthermore, osteoclast differentiation induced by 30 ng/ml RANKL is inhibited by more than 80% after treatment with 200 ng/ml of OPG, a decoy receptor and inhibitor of RANKL. FIG. 11A illustrates quantification of RAW264.7 cells treated with 30 ng/ml RANKL or conditioned media±200 ng/ml OPG with p<0.01 (**). Referring to FIG. 10A and FIG. 11A, it was observed that such treatment resulted in only ˜50% inhibition of CM induced osteoclast differentiation. These results indicated that additional factor(s), other than RANKL, in the tumor CM play an important role in enhancing osteoclastogenesis. A number of RANKL-independent osteoclast differentiation factors have been previously described, such as IL-6, MIF-1α, PTHrP, TNFα, GM-CSF and IGF (Weilbaecher et al., 2011). To examine whether any of these or other cytokines were secreted by the metastatic cells to induce osteoclastogenesis, cytokine expression was evaluated in CM from the three series of cell lines with differential bone metastatic abilities. FIGS. 11A-11G illustrate that soluble ICAM1 in tumor conditioned media synergizes with RANKL to promote osteoclast differentiation. FIG. 11B illustrates heat map from cytokine array depicting relative protein expression in conditioned media from indicated cell lines as compared to their weakly metastatic counterparts (4T1 vs 4T1.2, TSU-Pr1 vs TSU-PR1-B2, and MDA-231 vs SCP28, respectively). Referring to this figure, it was observed that among the cytokines analyzed, only soluble ICAM1 (sICAM1), the cleaved extracellular domain of ICAM1, was consistently overexpressed in the highly metastatic cell lines. Cell surface ICAM1 has been previously implicated in osteoclast differentiation, and antibodies against ICAM1 reduce osteoclast maturation (Harada et al., 1998; Kurachi et al., 1993). However, the functional role and mechanism of tumor-derived sICAM1 in osteoclastogenesis is unknown. FIG. 11C illustrates quantification of TRAP⁺ osteoclasts from RAW264.7 cells treated with 50 ng/ml RANKL±50 ng/ml sICAM1 (dark gray bars) for 0, 1, 2, 3, 4, or 6 days compared to non-treated control (high gray bars), with p<0.001 (***). Referring to this figure, it was observed that treatment of RAW264.7 cells with 50 ng/ml sICAM1 and 50 ng/ml RANKL together prompted osteoclast differentiation within 2 days, while treatment with RANKL alone required approximately 4 days for the first mature osteoclasts to differentiate. FIG. 10C illustrates qRT-PCR mRNA expression levels of indicated genes in RAW264.7 cells treated with 30 ng/mL RANKL±sICAM1 for indicated length of time. Referring to FIG. 10C, analysis of osteoclast marker genes confirmed that sICAM1 treatment together with 50 ng/ml RANKL reduced the time necessary for osteoclast differentiation compared to RANKL treatment alone, although there was no difference in endpoint expression levels for any of the mRNAs. FIG. 11D illustrates quantification of TRAP⁺ osteoclasts from RAW264.7 cells treated with 10 ng/ml RANKL, 50 ng/ml sICAM1, or both for 6 days with p<0.05 (*). FIG. 11E illustrates qRT-PCR analysis of selected miRNAs in RAW264.7 cells after indicated length of treatment with 10 ng/ml RANKL or/and 50 ng/ml sICAM1. The results were statistically significant with p<0.05 (*) assessed by analysis of variance (ANOVA) for all miRNAs. Referring to FIGS. 11D and 11E, it was observed that although sICAM1 alone was not sufficient to induce differentiation, it exhibited an additive effect on differentiation when combined with limiting concentrations of RANKL (10 ng/ml RANKL, FIG. 11D), and a significant inhibition in the expression of miRNAs downregulated during osteoclastogenesis, which was magnified after co-treatment with RANKL (FIG. 11E). Since the ELISA results from CM samples revealed very low RANKL concentrations that are insufficient to support osteoclast maturation, and treatment with sICAM1 and RANKL revealed an additive effect, that CM-induced differentiation was hypothesized to be due to the combined effect of both secreted factors.

To test this, RAW264.7 cells were treated with 4 ng/ml RANKL (equivalent to the level in tumor CM) and increasing concentrations of sICAM1. FIG. 10D illustrates representative images of RAW264.7 cells treated with Ong/ml RANKL plus indicated concentration of sICAM1 for 6 days as in FIG. 11F. Scale bar represents 400 μm. FIG. 11F illustrates quantification of osteoclasts/field and nuclei/osteoclast in RAW264.7 cells treated with 4 ng/ml RANKL plus indicated concentration of sICAM1 for 6 days. Results were statistically significant with p<0.01 (**) and p<0.001 (***).

Referring to FIG. 10D and FIG. 11F, it was observed that while low levels of sICAM1 had no effect on RAW264.7 cells in the presence of 4 ng/ml RANKL, high levels of TRAP⁺ osteoclasts were observed after treatment with 50 ng/ml sICAM1, approaching that of CM treatments.

FIG. 10E illustrates representative images of RAW264.6 cells treated with 30 ng/ml RANKL or SCP28 CM±50 μg/ml ICAM1 antibody. FIG. 11G illustrates quantification of TRAP⁺ osteoclasts from RAW264.7 cells treated with 30 ng/ml RANKL or SCP28 conditioned media followed by treatment with indicated concentration of ICAM1 antibody (in μg/mL). Data in the figure represent average ±SEM; p values were based on Student's t-test unless otherwise indicated. Results were statistically significant with p<0.05 (*), and p<0.01 (**) Referring to FIG. 10E and FIG. 11G, it was observed that when RAW264.7 cells were cultured with a function blocking monoclonal antibody (mAb) against ICAM1, they exhibited a dose-dependent decrease in osteoclast differentiation induced by SCP28 CM, while RANKL-induced osteoclastogenesis was unaffected.

FIG. 10F illustrates representative images and quantification of RAW264.7 cells treated with SCP28 CM±50 ng sICAM1 antibody±200 ng/mL OPG and p<0.05 (*). FIG. 10G illustrates quantification and representative images of bone resorption in RAW264.7 cells treated with SCP28 CM±50 ng/ml sICAM1 antibody. These results were statistically significant with p<0.05 (*). Referring to FIGS. 10F and 10G, it was observed that treating SCP28 CM with sICAM1 mAb resulted in an additive inhibitory effect when combined with OPG (FIG. 10F), and ICAM1 mAb was able to inhibit the bone resorbing activity of CM-treated RAW264.7 cells (FIG. 10G).

FIG. 10H illustrates RAW264.7 cell chemotaxis to indicated treatment of sICAM1 or SCP28 CM, with or without treatment of 50 ng/ml sICAM1 antibody. These results were significant with p<0.05 (*). Referring to this figure, it was observed that RAW264.7 cells treated with sICAM1 showed increased migration in a dose dependent manner, which was once again inhibited by the ICAM1 mAb. Taken together, these results support a functional role for sICAM1 as a crucial component of tumor CM in stimulating osteoclastogenesis.

Previous studies have identified αLβ2 and αMβ2 integrins as receptors of sICAM1 (Carlos and Harlan, 1994). FIG. 10I illustrates representative images of RAW264.7 cells treated with RANKL or SCP28 CM±20 ng ICAM1 antibody or 20 ng β2 integrin antibody. Scale bar represents 200 μm. Data in the figure represent average ±SEM; p values were based on Student's t test.

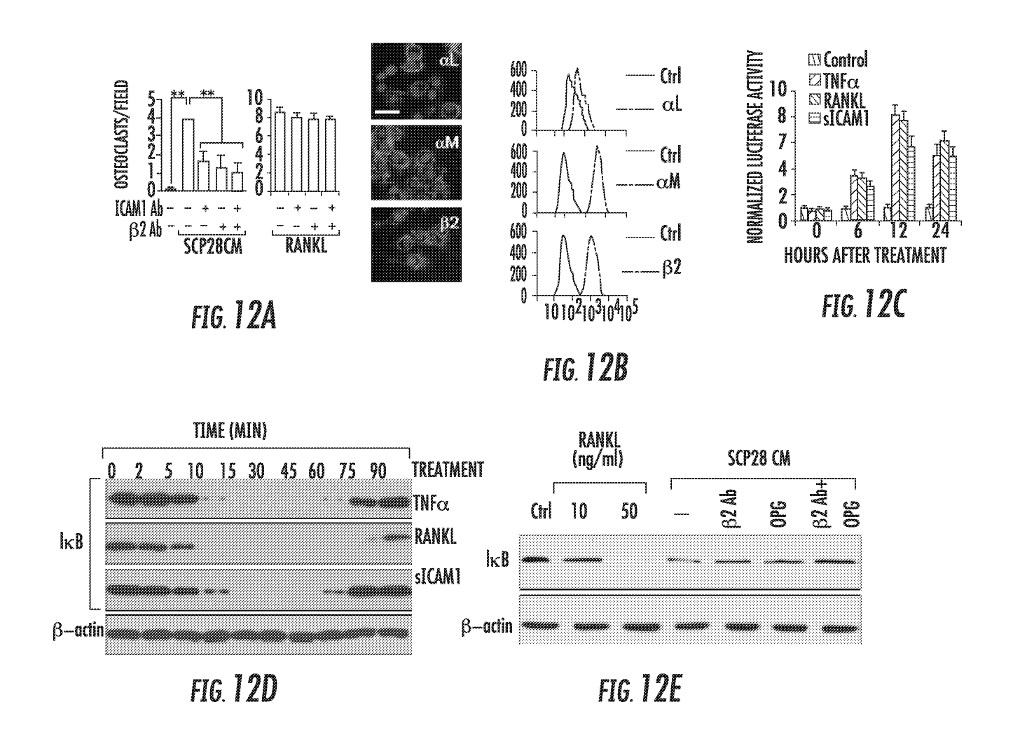

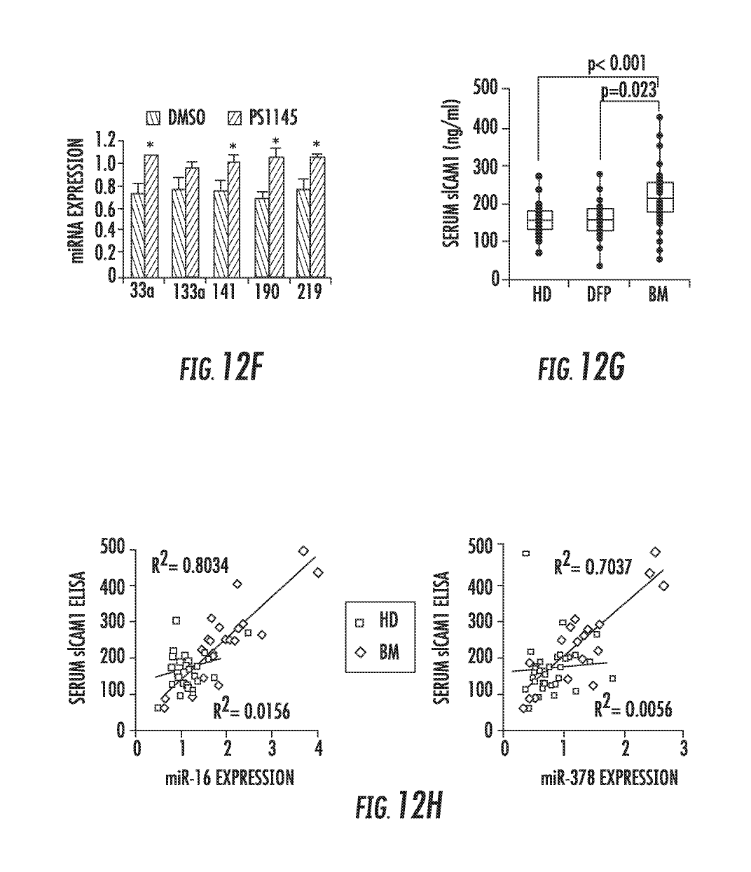

FIGS. 12A to 12H illustrate that sICAM1 promotes osteoclastogenesis by activating NFκB signaling, and sICAM1 levels in patient serum samples are correlated with miR-16 and miR-378 levels, and associated with bone metastasis. FIG. 12A illustrates quantification of TRAP⁺ osteoclasts from RAW264.7 cells treated with RANKL or SCP28 conditioned media±ICAM1 antibody or 20 μg β2 integrin antibody. Data represent average ±SEM. These results were statistically significant with p<0.01 (**) assessed by Student's t-test. Referring to FIG. 12A and FIG. 10I, it was observed that a function blocking antibody against the β2 subunit inhibited SCP28 CM, but not RANKL, -induced differentiation.

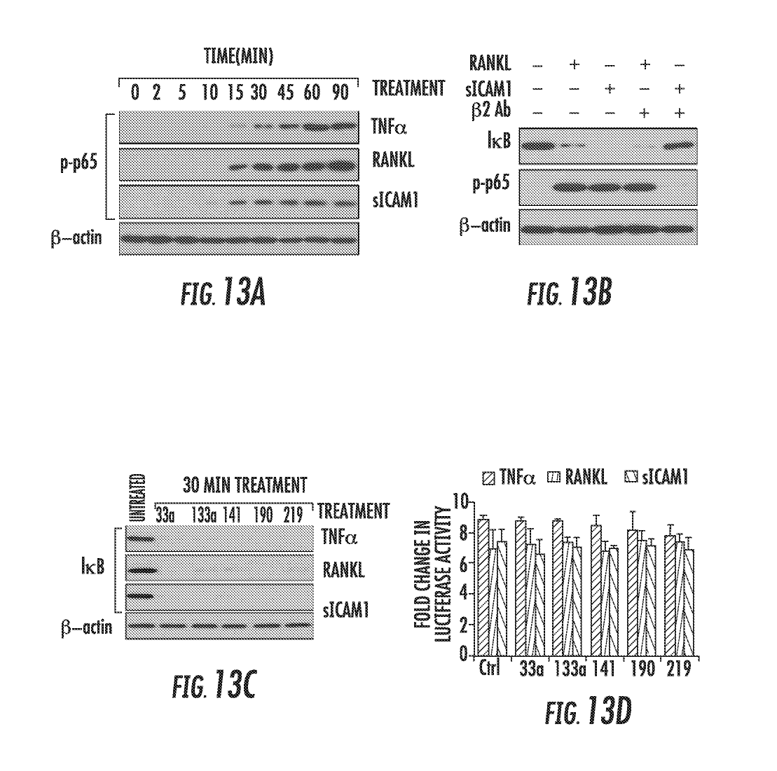

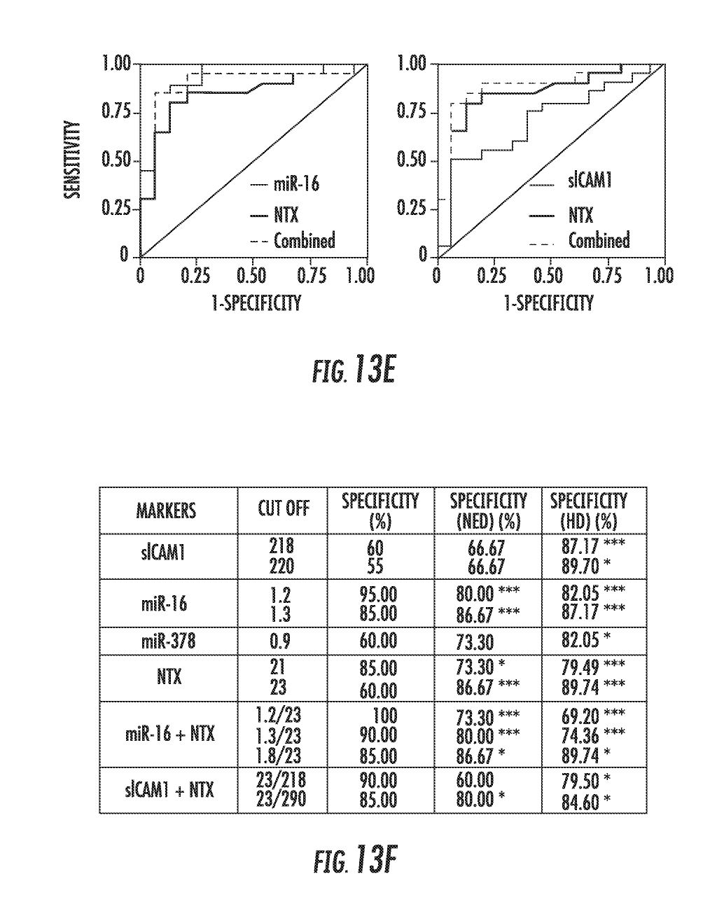

FIG. 12B illustrates expression of αL, αM, and β2 integrin subunits on RAW264.7 by IF (left) and flow cytometry (right). Scale bar represents 25 μm. Referring to FIG. 12B, analysis of RAW264.7 cells revealed strong expression of αM and β2 subunits on the cell surface, and lower expression of αL. Signaling through β2 integrins has been previously shown to activate the NFκB pathway in neutrophils under stimulation by GM-CSF or IL-6 (Kettritz et al., 2004), although it is not known if sICAM1 can similarly activate NFκB. FIG. 12C illustrates normalized activity of NFκB luciferase reporter in RAW264.7 cells after indicated treatment with TNFα (gray bar), RANKL (black bar), or sICAM1 (diagonally stripped bar) and control (light gray bar) for 0, 6, 12, or 24 hours. Data represent average ±SEM. The highest level of luciferase activity was observed 12 hours for all treatments. FIG. 12D illustrates Western blot analysis of IκB in RAW264.7 cells after treatment with TNFα, RANKL, or sICAM1 for 0, 2, 5, 10, 15, 30, 45, 60, 75, or 90 minutes. Referring to this figure, it was observed that degradation of IκB degraded over time for all treatments. FIG. 12E illustrates Western blot analysis of IκB in RAW264.7 cells after treatment with indicated concentration of RANKL or SCP28 CM±20 ng 132 antibody or 200 ng/ml OPG for 2 hours. FIGS. 13A-13G illustrate functional and clinical significance of sICAM1 and osteoclast miRNAs in bone metastasis. FIG. 13A illustrates Western blot analysis of phosphorylated-p65 in RAW264.7 cells after treatment with TNFα, RANKL, or sICAM1 for 0, 2, 5, 10, 15, 30, 45, 60 or 90 minutes. Phosphorylated-p65 was observed after 15 minutes of treatment for all treatments. Referring to FIGS. 12C, 12D and 12E, and FIG. 13A, activation of the pathway was examined by monitoring the activation of an NFκB luciferase reporter (FIG. 12C), degradation of IκBα (FIG. 12D), or phosphorylation of p65 (FIG. 13A) in RAW264.7 cells treated with TNFα, RANKL, or sICAM1 over time and observed similar responses in these three treatment conditions. Referring to FIG. 12E, it was observed that SCP28 CM treatment also showed a decrease in IκBα, which was inhibited by the addition of an antibody against the β2 integrin or OPG.

FIG. 13B illustrates Western blot analysis of IκB or p-p65 in RAW264.7 cells after treatment with RANKL, sICAM1 or β2 integrin antibody. Referring to this figure, it was observed that the β2 integrin antibody inhibited sICAM1, but not RANKL, induced activation of NΓKB. FIG. 12F illustrates relative qRT-PCR miRNA expression level in RAW264.7 cells treated with 50 ng/ml sICAM1 (dark gray bar), with or without 20 μM IKK inhibitor PS1145 (light gray bar) for 6 days, after normalization to the expression level without sICAM1 treatment. Data represent average ±SEM. The results were statistically significant with p<0.05 (*). Referring to this figures, it was observed that addition of PS1154 resulted in higher levels of miRNA expression for all miRNA tested. FIG. 13C illustrates Western blot analysis of IκB in HeLa cells after transient transfection with miR-33a, miR-133a, miR-141, miR-190 and miR-219, followed by 30 minute treatment with TNFα, RANKL, or sICAM-1. Referring to this figure, degradation of IκB was observed for all treatments. Referring to FIG. 12F and FIGS. 13C and 13D, it was observed that while sICAM1-induced miRNA changes were abrogated after RAW264.7 cells were treated with the IKK inhibitor PS1145 (FIG. 12F), miRNA transfection did not affect NFκB activation induced by TNFα, RANKL, or sICAM1 (FIGS. 13C and 13D), showing that these miRNAs act downstream of NFκB activation to influence osteoclastogenesis. Together, these results implicate sICAM1 as a bone metastatic cell-secreted factor that functions through β2 integrin and NFκB signaling to enhance osteoclast differentiation in the presence of low levels of RANKL.

### Example 9

### Correlation of Serum sICAM1 and miRNA Expression in Patients with Bone Metastasis

To further investigate the clinical significance of sICAM1 as a tumor-derived factor in inducing osteoclastogenesis and associated miRNA changes during bone metastasis, the expression levels of sICAM1 were analyzed in serum samples collected from healthy female donors (HD), disease-free breast cancer patients showing no occurrence of bone metastasis (DFP, samples taken immediately following resection of the primary tumor), or breast cancer patients with bone metastasis (BM).

FIG. 12G illustrates ELISA quantification of sICAM1 expression levels in serum samples collected from healthy female donors (HD, n=41), disease-free breast cancer patients (DFP, n=16), or breast cancer patients with bone metastasis (BM, n=38). p values based on Mann-Whitney test. Referring to this figure, it was observed that patients with bone metastases exhibited significantly increased serum sICAM1 level compared to healthy donors or disease free patients.

FIG. 12H illustrates correlation of qRT-PCR miR-16 or miR-378 expression levels with ELISA sICAM1 protein expression levels in serum samples from healthy donors or bone metastasis patients from FIG. 12G. Referring to this figure, a strong correlation was seen between serum expression of sICAM1 and miR-16 or miR-378 in bone metastasis patients, while little correlation was seen in samples from healthy donors.

To further investigate the diagnostic potential of miR-16 and miR-378 as secreted biomarkers for osteolytic bone metastasis, the serum expression of the miRNAs was compared against N-terminal telopeptide (NTX), a standard marker of bone turnover. The sensitivity and specificity of using serum levels of miR-16, miR-378, sICAM1 and NTX, alone or in combination, for detecting bone metastasis were determined (FIGS. 13E and 13F). FIG. 13E illustrates ROC curves for the diagnosis of breast cancer bone metastasis using miR-16, sICAM1, NTX, or combinations of markers. FIG. 13F illustrates sensitivity and specificity values for diagnosis of bone metastasis relative to patients with no evidence of disease (NED) or healthy donors (HD) for indicated markers. The results were statistically significant with p<0.05(*), p<0.001 (***) assessed by chi-square test. Referring to FIGS. 13E and 13F, it was observed that the diagnostic values of miR-378 and sICAM1 were lower than that of either miR-16 or NTX as individual variables, and combining sICAM1 with NTX did not significantly increase the value. In contrast, miR-16 revealed increased specificity over NTX, while maintaining a similar sensitivity. The combination of miR-16 with NTX produced increased sensitivity, with only a slight reduction in specificity. These findings underscored the clinical significance of elevated sICAM1 and miR-16/-378 expression in the serum of bone metastasis, and suggest their potential use as biomarkers for diagnosis of bone metastasis or predictive marker for anti-ICAM1 therapeutics.

### Example 10

### Clinical Applications

Inhibition of osteoclast differentiation and osteolytic bone metastasis was demonstrated after ectopic expression of several miRNAs down-regulated during osteoclastogenesis. sICAM1 was identified as a tumor-secreted factor that enhances osteoclast differentiation and influences osteoclast miRNA expression via the NFκB pathway. It was observed that serum levels of both sICAM1 and miRNAs are indicators of bone metastasis burden in breast cancer patients. These observations suggest multiple avenues for clinical translation of the findings.

A microarray-based analysis of miRNA expression changes in mouse primary osteoclasts was conducted 24 h after M-CSF/RANKL treatment and revealed altered expression of dozens of miRNAs. miRNA changes induced by both RANKL and tumor conditioned media were examined, and samples were collected after 7 days of treatment with RANKL or CM, after full osteoclast differentiation was observed (FIG. 13C). Such analysis may show not only miRNAs involved in regulating osteoclast differentiation, but also those that influence the function of mature osteoclasts. Additionally, a distinct set of most differentially expressed miRNAs were identified in our current study. Three different human and mouse cancer cell lines were used from two different tumor types in the CM treatment experiments. Despite the fact that these three distinctively different tumor cells all secrete a sub-minimal amount of RANKL into their CM, miRNA expression changes stimulated by their CM showed high levels of consistency with RANKL-induced osteoclastogenesis. Ectopic overexpression of down-regulated miRNAs inhibited osteoclastogenesis induced by multiple conditions, including RANKL, tumor CM, and Jagged1, in multiple osteoclast cell lines or primary preosteoclasts. These findings support a highly conserved miRNA regulatory network that controls osteoclast differentiation in both pathological and physiological conditions.

Examination of the target genes of osteoclast-inhibiting miRNAs revealed direct targeting a number of known osteoclast genes, including Mitf, Calcr, Traf6, and Mmp14. Decreased expression of Mitf, Traf6, Calcr, or Mmp14 is likely to constitute a miRNA target gene network responsible for the defect seen in osteoclast differentiation after ectopic expression of these miRNAs. Analysis of stage-specific markers of osteoclast differentiation revealed that miR-133a, miR-141, and miR-219 may inhibit early osteoclastogenesis, while miR-190 may inhibit osteoclast differentiation or function after commitment to an osteoclast fate.

CM from highly bone metastatic cells was able to induce osteoclast differentiation and miRNA expression changes despite the presence of very low levels of RANKL, which is insufficient to induce osteoclastogenesis. sICAM1 was identified as a tumor-derived factor in the CM that enhances osteoclast activation at the minimal concentration of RANKL. ICAM1 has been previously observed to increase in expression level during osteoclast differentiation, while ablation of ICAM1 inhibited osteoclast differentiation from peripheral blood mononuclear cells (Nakano et al., 2004). However, it was previously unknown how tumor-derived sICAM1 influences osteoclast differentiation. The observation that sICAM1 is capable of increasing RANKL induced osteoclast differentiation reveals an additional mechanism for osteoclast regulation from bone metastatic cells and lead to a potential target for therapeutic intervention. Examples herein indicate that the tumor secreted ICAM1 induced osteoclast differentiation, which is insufficient for independently inducing osteoclastogenesis but capable of enhancing differentiation in the presence of low, but physiological, levels of RANKL. It was observed that sICAM1 binding to its cognate receptor 132 integrins activates NFκB signaling, which is essential for canonical, RANK-mediated osteoclast differentiation (Boyle et al., 2003; Teitelbaum and Ross, 2003), and may explain the mechanism by which sICAM1 enhances osteoclast differentiation. Although it is not known how 132 integrins activate the NFκB pathway, it has been proposed that they provide a co-stimulatory effect on NFκB signaling in neutrophils treated with GM-CSF or IL-8 (Kettritz et al., 2004). Furthermore, 132 integrin clustering can activate NFκB (Kim et al., 2004). sICAM1-induced alterations in miRNA expression appear to occur downstream of NFκB, as inhibiting the pathway prevented miRNA changes, while none of the miRNAs examined in this study altered NFκB signaling. Additionally, sICAM1 increases the migration of pre-osteoclast cells, which may indicate a role for bone-metastasis derived sICAM1 in the recruitment of osteoclasts to the developing bone lesion.

Several avenues for potential translational applications in the clinical management of bone metastasis were revealed herein. Intravenous injection of pre-miR-133a, -141, -190, or -219 significantly reduced osteoclast activity in vivo. These effects were seen most clearly in the trabecular region of the femur and tibia, while no significant difference was seen in cortical bone thickness. This finding is similar to the observation in animals treated with bisphosphonates (Quattrocchi et al., 2012), possibly because of the higher rates of turnover in the trabecular bone. Consistently, treatment of mice with the pre-miR-141 and -219 was capable of inhibiting osteolytic bone metastasis. It is curious that miR-133a and miR-190 had no measurable effect on bone metastasis, despite a substantial influence on normal bone remodeling. It seems unlikely that this is due to a tumor-intrinsic mechanism, since in vitro studies showed no effect on SCP28 growth or survival after miRNA treatment. Instead, is possible that this is explained by the measured differences in stability of the miRNAs in circulation, as inhibition of bone metastasis may require greater levels of miRNAs to reach the bone than inhibition of normal bone remodeling.

It was observed herein that systemic injection of unconjugated miRNAs was sufficient to induce broad changes in bone remodeling and appeared to be well tolerated by the mice. While data herein from in vitro and in vivo experiments illustrate miRNA-mediated inhibition of osteoclasts, it is possible that these miRNAs might also target additional cells in vivo. Therefore, additional further analyses of other potential cellular and molecular targets of these miRNAs during long-term treatment in vivo should be conducted when developing potential miRNA-based therapeutic applications. It is possible that improved delivery methods might enhance the pharmacokinetics and efficacy of the miRNAs on bone metastasis and potentially reveal an effect from miR-133a and miR-190. The therapeutic effects of miR-141 and miR-219 mimic those seen in mice after treatment with ZOMETA™. In addition, an additive effect from combined treatment with miR-141/-190/-219 and ZOMETA™ was noted on bone resorption in vitro. Thus, it is possible that combinatorial treatments including miRNAs and currently approved osteoclast-targeting agents such as biosphosphonates and denosumab (RANKL antibody) might provide enhanced clinical efficacy. Furthermore, the functional role of tumor-derived sICAM1 in pathological osteoclast differentiation suggests the potential for using ICAM1 blocking antibodies for targeted therapeutics. Therefore, osteoclast miRNAs and sICAM1 represent potential targets in the treatment of aberrant osteoclast activity, namely bone degenerative diseases such as osteolytic bone metastasis, osteoporosis and Paget's disease. The finding that miR-16 and miR-378 levels increase in bone lesions and serum samples of bone metastasis patients presents the potential for their use as metastasis biomarkers. In particular, combined miR-16 and NTX as biomarkers increased the sensitivity of bone metastasis diagnosis, although the potential clinical application of this combination still awaits further large scale prospective analysis.

### REFERENCES

Bartel, D. P. (2009). MicroRNAs: target recognition and regulatory functions. Cell 136, 215-233.
Blanco, M. A., LeRoy, G., Khan, Z., Aleckovic, M., Zee, B. M., Garcia, B. A., and Kang, Y. (2012). Global secretome analysis identifies novel mediators of bone metastasis. Cell research 22, 1339-1355.
Boyle, W. J., Simonet, W. S., and Lacey, D. L. (2003). Osteoclast differentiation and activation. Nature 423, 337-342.
Carlos, T. M., and Harlan, J. M. (1994). Leukocyte-endothelial adhesion molecules. Blood 84, 2068-2101.
Chaffer, C. L., Dopheide, B., McCulloch, D. R., Lee, A. B., Moseley, J. M., Thompson, E. W., and Williams, E. D. (2005). Upregulated MT1-MMP/TIMP-2 axis in the TSU-Pr1-B1/B2 model of metastatic progression in transitional cell carcinoma of the bladder. Clin Exp Metastasis 22, 115-125.
Clezardin, P. (2011). Therapeutic targets for bone metastases in breast cancer. Breast Cancer Res 13, 207.
Croce, C. M. (2009). Causes and consequences of microRNA dysregulation in cancer. Nat Rev Genet 10, 704-714.
Davey, R. A., Turner, A. G., McManus, J. F., Chiu, W. S., Tjahyono, F., Moore, A. J., Atkins, G. J., Anderson, P. H., Ma, C., Glatt, V., et al. (2008). Calcitonin receptor plays a physiological role to protect against hypercalcemia in mice. J Bone Miner Res 23, 1182-1193.
Ell, B., and Kang, Y. (2012). SnapShot: Bone Metastasis. Cell 151, 690-690 e691.
Flamini, E., Mercatali, L., Nanni, O., Calistri, D., Nunziatini, R., Zoli, W., Rosetti, P., Gardini, N., Lattuneddu, A., Verdecchia, G. M., and Amadori, D. (2006). Free DNA and carcinoembryonic antigen serum levels: an important combination for diagnosis of colorectal cancer. Clin Cancer Res 12, 6985-6988.
Gonzalo, P., Guadamillas, M. C., Hernandez-Riquer, M. V., Pollan, A., Grande-Garcia, A., Bartolome, R. A., Vasanji, A., Ambrogio, C., Chiarle, R., Teixido, J., et al. (2010). MT1-MMP is required for myeloid cell fusion via regulation of Racl signaling. Dev Cell 18, 77-89.
Grimson, A., Farh, K. K., Johnston, W. K., Garrett-Engele, P., Lim, L. P., and Bartel, D. P. (2007). MicroRNA targeting specificity in mammals: determinants beyond seed pairing. Mol Cell 27, 91-105.
Harada, H., Kukita, T., Kukita, A., Iwamoto, Y., and Iijima, T. (1998). Involvement of lymphocyte function-associated antigen-1 and intercellular adhesion molecule-1 in osteoclastogenesis: a possible role in direct interaction between osteoclast precursors. Endocrinology 139, 3967-3975.
Hemingway, F., Taylor, R., Knowles, H. J., and Athanasou, N. A. (2011). RANKL-independent human osteoclast formation with APRIL, BAFF, NGF, IGF I and IGF II. Bone 48, 938-944.
Hershey, C. L., and Fisher, D. E. (2004). Mitf and Tfe3: members of a b-HLH-ZIP transcription factor family essential for osteoclast development and function. Bone 34, 689-696.
Hodgkinson, C. A., Moore, K. J., Nakayama, A., Steingrimsson, E., Copeland, N. G., Jenkins, N. A., and Arnheiter, H. (1993). Mutations at the mouse microphthalmia locus are associated with defects in a gene encoding a novel basic-helix-loop-helix-zipper protein. Cell 74, 395-404.
Holmbeck, K., Bianco, P., Caterina, J., Yamada, S., Kromer, M., Kuznetsov, S. A., Mankani, M., Robey, P. G., Poole, A. R., Pidoux, I., et al. (1999). MT1-MMP-deficient mice develop dwarfism, osteopenia, arthritis, and connective tissue disease due to inadequate collagen turnover. Cell 99, 81-92.
Kang, Y., Siegel, P. M., Shu, W., Drobnjak, M., Kakonen, S. M., Cordon-Cardo, C., Guise, T. A., and Massague, J. (2003). A multigenic program mediating breast cancer metastasis to bone. Cancer Cell 3, 537-549.
Kettritz, R., Choi, M., Rolle, S., Wellner, M., and Luft, F. C. (2004). Integrins and cytokines activate nuclear transcription factor-kappaB in human neutrophils. J Biol Chem 279, 2657-2665.
Kim, C. H., Lee, K. H., Lee, C. T., Kim, Y. W., Han, S. K., Shim, Y. S., and Yoo, C. G. (2004). Aggregation of beta2 integrins activates human neutrophils through the IkappaB/NF-kappaB pathway. J Leukoc Biol 75, 286-292.
Kloosterman, W. P., and Plasterk, R. H. (2006). The diverse functions of microRNAs in animal development and disease. Dev Cell 11, 441-450.
Korpal, M., Yan, J., Lu, X., Xu, S., Lerit, D. A., and Kang, Y. (2009). Imaging transforming growth factor-beta signaling dynamics and therapeutic response in breast cancer bone metastasis. Nat Med 15, 960-966.
Krek, A., Grun, D., Poy, M. N., Wolf, R., Rosenberg, L., Epstein, E. J.,
MacMenamin, P., da Piedade, I., Gunsalus, K. C., Stoffel, M., and Rajewsky, N. (2005). Combinatorial microRNA target predictions. Nat Genet 37, 495-500.
Kurachi, T., Morita, I., and Murota, S. (1993). Involvement of adhesion molecules LFA-1 and ICAM-1 in osteoclast development. Biochim Biophys Acta 1178, 259-266.
Le, X. F., Merchant, O., Bast, R. C., and Calin, G. A. (2010). The Roles of MicroRNAs in the Cancer Invasion-Metastasis Cascade. Cancer microenvironment: official journal of the International Cancer Microenvironment Society 3, 137-147.
Lelekakis, M., Moseley, J. M., Martin, T. J., Hards, D., Williams, E., Ho, P., Lowen, D., Javni, J., Miller, F. R., Slavin, J., and Anderson, R. L. (1999). A novel orthotopic model of breast cancer metastasis to bone. Clin Exp Metastasis 17, 163-170.
Lomaga, M. A., Yeh, W. C., Sarosi, I., Duncan, G. S., Furlonger, C., Ho, A., Morony, S., Capparelli, C., Van, G., Kaufman, S., et al. (1999). TRAF6 deficiency results in osteopetrosis and defective interleukin-1, CD40, and LPS signaling. Genes Dev 13, 1015-1024.
Lu, X., Mu, E., Wei, Y., Riethdorf, S., Yang, Q., Yuan, M., Yan, J., Hua, Y., Tiede, B. J., Haffty, B. G., et al. (2011). VCAM-1 promotes osteolytic expansion of indolent bone micrometastasis of breast cancer by engaging alpha4beta1-positive osteoclast progenitors. Cancer Cell 20, 701-714.
Mann, M., Barad, O., Agami, R., Geiger, B., and Hornstein, E. (2010). miRNA-based mechanism for the commitment of multipotent progenitors to a single cellular fate. Proc Natl Acad Sci USA.
Mizoguchi, F., Izu, Y., Hayata, T., Hemmi, H., Nakashima, K., Nakamura, T., Kato, S., Miyasaka, N., Ezura, Y., and Noda, M. (2010). Osteoclast-specific Dicer gene deficiency suppresses osteoclastic bone resorption. J Cell Biochem 109, 866-875.
Nakano, K., Okada, Y., Saito, K., and Tanaka, Y. (2004). Induction of RANKL expression and osteoclast maturation by the binding of fibroblast growth factor 2 to heparan sulfate proteoglycan on rheumatoid synovial fibroblasts. Arthritis Rheum 50, 2450-2458.
Quattrocchi, C. C., Dell′Aia, P., Errante, Y., Occhicone, F., Longo, D., Virzi, V., Tonini, G., Napoli, N., Santini, D., and Zobel, B. B. (2012). Differential effect of zoledronic acid on normal trabecular and cortical bone density in oncologic patients with bone metastases. J Bone Oncology 1, 24-29.
Sethi, N., Dai, X., Winter, C. G., and Kang, Y. (2011). Tumor-derived JAGGED1 promotes osteolytic bone metastasis of breast cancer by engaging notch signaling in bone cells. Cancer Cell 19, 192-205.
Sharma, S. M., Bronisz, A., Hu, R., Patel, K., Mansky, K. C., Sif, S., and Ostrowski, M. C. (2007). MITF and PU.1 recruit p38 MAPK and NFATc1 to target genes during osteoclast differentiation. J Biol Chem 282, 15921-15929.
Sugatani, T., and Hruska, K. A. (2007). MicroRNA-223 is a key factor in osteoclast differentiation. J Cell Biochem 101, 996-999.
Sugatani, T., and Hruska, K. A. (2009). Impaired micro-RNA pathways diminish osteoclast differentiation and function. J Biol Chem 284, 4667-4678.
Sugatani, T., Vacher, J., and Hruska, K. A. (2011). A microRNA expression signature of osteoclastogenesis. Blood.
Teitelbaum, S. L., and Ross, F. P. (2003). Genetic regulation of osteoclast development and function. Nat Rev Genet 4, 638-649.
Weilbaecher, K. N., Guise, T. A., and McCauley, L. K. (2011). Cancer to bone: a fatal attraction. Nat Rev Cancer 11, 411-425.
Weilbaecher, K. N., Motyckova, G., Huber, W. E., Takemoto, C. M., Hemesath, T. J., Xu, Y., Hershey, C. L., Dowland, N. R., Wells, A. G., and Fisher,
D. E. (2001). Linkage of M-CSF signaling to Mitf, TFE3, and the osteoclast defect in Mitf(mi/mi) mice. Mol Cell 8, 749-758.
Zhang, J., Zhao, H., Chen, J., Xia, B., Jin, Y., Wei, W., Shen, J., and Huang, Y. (2012). Interferon-beta-induced miR-155 inhibits osteoclast differentiation by targeting SOCS1 and MITF. FEBS Lett 586, 3255-3262.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A pharmaceutical composition comprising a therapeutic agent including a first nucleic acid with at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86), and at least one additional therapeutic agent.

2. The pharmaceutical composition of claim 1, wherein the first nucleic acid has a sequence selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86).

3. The pharmaceutical composition of claim 1, wherein the at least one additional therapeutic agent includes a second nucleic acid with at least 90% identity to a sequence is selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86), wherein the sequence selected for the second nucleic acid is different than the sequence selected for the first nucleic acid.

4. The pharmaceutical composition of claim 3, wherein the second nucleic acid has a sequences selected from the group consisting of: miR-33a (SEQ ID NO: 74), miR-133a (SEQ ID NO: 61), miR-190 (SEQ ID NO: 64), miR-219 (SEQ ID NO: 66), miR-141 (SEQ ID NO: 69), precursor miR-33a (SEQ ID NO: 77), precursor miR-133a (SEQ ID NO: 78), precursor miR-141 (SEQ ID NO: 79), precursor miR-190 (SEQ ID NO: 80), precursor miR-219 (SEQ ID NO: 81), precursor miR-33a(2) (SEQ ID NO: 82), precursor miR-133a(2) (SEQ ID NO: 83), precursor miR-141(2) (SEQ ID NO: 84), precursor miR-190(2) (SEQ ID NO: 85), and precursor miR-219(2) (SEQ ID NO: 86).

5. The pharmaceutical composition of claim 1, wherein the at least one additional therapeutic agent is selected from the group consisting of: a bone metastasis therapeutic agent, a breast cancer therapeutic agent, and an anti-estrogen therapeutic agent.

6. The pharmaceutical composition of claim 5, wherein the at one additional therapeutic agent is selected from the group consisting of: bisphosphonates, alendronate, ibandronate, risedronate, pamidronate, a RANKL antibody, a sclerostin antibody, methotrexate, paclitaxel, or paclitaxel albumin-stabilized nanoparticle formulation, doxorubicin or doxorubicin hydrochloride, fluorouracil or 5-fluorouracil, everolimus, anastrozole, docetaxel, epirubicin hydrochloride, toremifene, fulvestrant, letrozole, gemcitabine hydrochloride, ixabepilone, megestrol acetate, cyclophosphamide, toremifene, lapatinib or lapatinib ditosylate, capecitabine, zoledronic acid, goserelin acetate, exemestane, tamoxifen, trastuzumab, ado-trastuzumab emtansine, and pertuzumab.

7. The pharmaceutical composition of claim 1, wherein the at least one additional therapeutic agent includes an antibody that specifically binds sICAM1.

8. The pharmaceutical composition of claim 1, wherein the at least one additional therapeutic agent includes an antibody that specifically binds an sICAM1 receptor.

9. The pharmaceutical composition of claim 8, wherein the sICAM1 receptor includes a β2 integrin.

10. The pharmaceutical composition of claim 9, wherein the β2 integrin is one of αL@2 integrin and αMβ2 integrin

11. The pharmaceutical composition of claim 7, wherein the antibody is a monoclonal antibody.

12. The pharmaceutical composition of claim 1 further comprising a pharmaceutically acceptable carrier.

13. The pharmaceutical composition of claim 1 further comprising a nanocarrier.

14. The pharmaceutical composition of claim 13, wherein the nanocarrier is selected from the group consisting of: lipid nanoparticles, liposomes, polymer particles, ligands and polydexstrins.

15. A method of treating a subject suffering from a bone degeneration disease comprising administering a pharmaceutical composition of claim 1 to the subject.

16. The method of claim 15, wherein the bone degeneration disease is selected from the group consisting of: bone metastasis, osteoporosis, and Paget disease.

17. The method of claim 16, wherein the bone degeneration disease is bone metastasis.

18. The method of claim 17 further comprising assessing inhibition of bone metastasis growth in the subject before and after treatment.

19. The method of claim 18, wherein the assessing includes measuring a rate of bone metastasis.

20. The method of claim 18, wherein the assessing includes determining expression of at least one osteoclast differentiation marker gene in the subject after treatment.

21. The method of claim 20, wherein the at least one osteoclast differentiation marker gene is selected from the group consisting of: Mitf, Traf6, Mmp14, Calcr, Cpr, Mmp9, Itgav, Oscar, Nfatc1, Ctsk, Calcr site1, Calcr site2, Mitf site1, and Mitf site 2.

22. The method of claim 18 further comprising terminating treatment if the bone metastasis growth in the subject is inhibited.

23. The method of claim 15, wherein the pharmaceutical composition is associated with a nanocarrier.

24. The method of claim 23, wherein the nanocarrier is selected from the group consisting of: lipid nanoparticles, liposomes, polymer particles, ligands and polydexstrins.

25. The method of claim 15, wherein the step of administering includes administering by a route selected from the group consisting of: intravenous, intraperitoneal, intramuscular, and subcutaneous injection.

26. The method of claim 15 further comprising treating the subject with radiation therapy.

27. The method of claim 15 further comprising treating the subject with immune therapy.

28. The method of claim 15, wherein the subject is a mammal.

29. The method of claim 28, wherein the mammal is a rodent.

30. The method of claim 28, wherein the subject is a human.

31. A method of treating a subject suffering from a bone degenerative disease comprising:
obtaining a test sample from the subject,
determining an expression level of at least one osteoclast differentiation marker in the test sample, wherein the at least one osteoclast differentiation marker is one or more of miRNAs or sICAM1, and
comparing the expression level of the at least one osteoclast differentiation marker in the test sample to the expression level of the at least one osteoclast differentiation marker in a reference sample, wherein an increase in the expression level of the at least one osteoclast differentiation marker in the test sample compared to the reference sample indicates the subject will be a positive responder, and a finding of a decrease in the expression level of the at least one osteoclast differentiation marker in the test sample compared to the reference sample indicates the subject will be a negative responder;
the method further comprising one or more of recommending or conducting at least one of:
treating the subject with the pharmaceutical composition of claim 1 if the subject the subject is determined to be a positive responder, and treating the subject with one or more of radiation therapy or immunotherapy if the subject is determined to be a negative responder.

32. The method of claim 31, wherein if the expression level of the one or more miRNAs is elevated compared to the reference, the step of treating the subject with the pharmaceutical composition of claim 1 is treating the subject with the pharmaceutical composition of claim 3.

33. The method of claim 31, wherein if the expression level of sICAM1 is elevated compared to the reference, the step of treating the subject with the pharmaceutical composition of claim 1 includes treating the subject with the pharmaceutical composition of claim 7.

34. The method of claim 31, wherein a correspondent osteoclast differentiation marker is an osteoclast differentiation marker of a same identity of one being compared in a test sample, and the reference sample is selected from the group consisting of: the expression level of the correspondent osteoclast differentiation marker in a second test sample obtained from a healthy individual, a reference value, and the expression level of the correspondent osteoclast differentiation marker in a second test sample that has been exposed to an agent inducing osteoclast differentiation.

35. The method of claim 34, wherein the agent is selected from the group consisting of: a tumor-conditioned medium, a receptor activator of NF-κB ligand, and sICAM1.

36. The method of claim 35, wherein the at least one osteoclast differentiation marker is selected from the group consisting of: miR-33a, miR-133a, miR-141, miR-190, mi-R-219, and sICAM1.

37. The method of claim 36, wherein the at least one osteoclast differentiation marker is selected from the group consisting of: miR-33a, miR-133a, miR-141, miR-190, and miR-219.

38. The method of claim 37, wherein the at least one osteoclast differentiation marker includes a nucleic acid having at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), and miR-141 (SEQ ID NO: 69).

39. The method of claim 31, wherein the correspondent osteoclast differentiation marker is an miRNA, and the miRNA is miR-16, miR-378, or a combination of mi-R-16 and miR-378.

40. The method of claim 39, wherein the one or more miRNA includes a nucleic acid having at least 90% identity to a sequence of SEQ ID NO: 75) or SEQ ID NO: 76.

41. The method of claim 31, wherein the subject is a mammal, and the mammal is selected from the group consisting of a rodent, a human, a primate, and a high value agricultural animal.

42. The method of claim 31, wherein the test sample comprises cells and serum.

43. A method for diagnosing whether a subject has bone metastasis comprising:
obtaining a test sample from the subject afflicted with cancer;
determining an expression level of at least one osteoclast differentiation marker in the test sample;
comparing the expression level of the at least one osteoclast differentiation marker in the test sample to the expression level of the corresponding osteoclast differentiation marker in a reference sample; and
diagnosing the subject as having bone metastasis if the expression level of the at least one osteoclast differentiation marker in the test sample is elevated compared to the corresponding osteoclast differentiation marker in a reference sample.

44. The method of claim 43, wherein the at least one osteoclast differentiation marker is selected from the group consisting of: miR-33a, miR-133a, miR-141, miR-190, mi-R-219, and sICAM1.

45. The method of claim 44, wherein the at least one osteoclast differentiation marker is selected from the group consisting of: miR-33a, miR-133a, miR-141, miR-190, and miR-219.

46. The method of claim 45, wherein the at least one osteoclast differentiation marker includes a nucleic acid having at least 90% identity to a sequence selected from the group consisting of: miR-33a (SEQ ID NO: 74), miR-133a (SEQ ID NO: 61), miR-190 (SEQ ID NO: 64), miR-219 (SEQ ID NO: 66), and miR-141 (SEQ ID NO: 69).

47. The method of claim 43, wherein the at least one osteoclast differentiation marker is an miRNA, and the miRNA is miR-16, miR-378, or a combination of mi-R-16 and miR-378.

48. The method of claim 47, wherein the one or more miRNA includes a nucleic acid having at least 90% identity to a sequence of SEQ ID NO: (SEQ ID NO: 75) or SEQ ID NO: (SEQ ID NO: 76).

49. The method of claim 43, wherein a correspondent osteoclast differentiation marker is an osteoclast differentiation marker of a same identity of one being compared in a test sample, and the reference sample is selected from the group consisting of: the expression level of the correspondent osteoclast differentiation marker in a second test sample obtained from a healthy individual, a reference value, and the expression level of the correspondent osteoclast differentiation marker in a second test sample that has been exposed to an agent inducing osteoclast differentiation.

50. The method of claim 43 further comprising recommending treating the subject having bone metastasis with a pharmaceutical composition of claim 1.
